(19) United States
(12) Patent Application Publication
(10) Pub. No.: US 2019/0135875 A1
(43) Pub. Date: May 9, 2019

(54) TRI-SEGMENTED PICHINDE VIRUSES AS VACCINE VECTORS

(71) Applicants: Hookipa Biotech GmbH, Vienna (AT); Universität Basel, Basel (CH)

(72) Inventors: Weldi Bonilla, Binningen (CH); Daniel David Pinschewer, Binningen (CH); Klaus Orlinger, Wien (AT)

(21) Appl. No.: 16/302,086

(22) PCT Filed: May 17, 2017
(86) PCT No.: PCT/EP2017/061865

§ 371 Date: Nov. 15, 2018

Related U.S. Application Data
(60) Provisional application No. 62/338,400, filed on May 18, 2016.

(51) Int. Cl. C07K 14/005

(52) U.S. Cl. CPC C07K 14/005; C12N 2760/10022; A61K 39/00; C12N 2760/10034; C12N 2760/10023

(57) ABSTRACT

The present application relates to Pichinde viruses with rearrangements of their open reading frames (“ORF”) in their genomes. In particular, described herein is a modified Pichinde virus genomic segment, wherein the Pichinde virus genomic segment is engineered to carry a viral ORF in a position other than the wild-type position of the ORF. Also described herein are trisegmented Pichinde virus particles comprising one L segment and two S segments or two L segments and one S segment. The Pichinde virus, described herein may be suitable for vaccines and/or treatment of diseases and/or for the use in immunotherapies.

This application claims the benefit of U.S. Provisional Application No. 62/338,400 filed May 18, 2016, which is hereby incorporated by reference in its entirety.

### REFERENCE TO SEQUENCE LISTING SUBMITTED ELECTRONICALLY

This application incorporates by reference a Sequence Listing submitted with this application as text file entitled “Sequence_Listing_13194-020-228.TXT” created on May 16, 2017 and having a size of 61,423 bytes.

### 1. INTRODUCTION

The present application relates to Pichinde viruses with rearrangements of their open reading frames (“ORF”) in their genomes. In particular, described herein is a modified Pichinde virus genomic segment, wherein the Pichinde virus genomic segment is engineered to carry a viral ORF in a position other than the wild-type position of the ORF. Also described herein are tri-segmented Pichinde virus particles comprising one L segment and two S segments or two L segments and one S segment. The Pichinde virus, described herein may be suitable for vaccines and/or treatment of diseases and/or for the use in immunotherapies.

### 2. BACKGROUND

### 2.1 Pichinde Virus General Background and Genomic Organization

Pichinde virus is an arenavirus isolated from Oryzomys albigularis (rice rats) in Columbia (reviewed in McLay et al, 2014, Journal of General Virology, 95: 1-15). Pichinde virus is nonpathogenic and is generally not known to cause diseases in humans. Serological evidence suggest a very low seroprevalence even in local human population (Trapido et al, 1971, Am J Trop Med Hyg, 20: 631-641). The family Arenaviridae is classified into two groups: the Old World (OW) arenaviruses such as Lassa fever virus (LASV) and Lymphocytic Choriomeningitis Virus (LCMV), and the New World (NW) arenaviruses such as Pichinde virus and Junin virus (Buchmeier et al, 2001, Arenaviridae: The Viruses and Their Replication, Fields Virology Vol 2, 1635-1668). Arenaviruses are enveloped RNA viruses. Their genome consists of two segments of single-stranded RNA of negative sense (FIG. 1A) (McLay et al, 2014, Journal of General Virology, 95: 1-15). Each segment encodes for two viral genes in opposite orientations. The short segment (S segment) encodes the viral glycoprotein (GP) and the nucleoprotein (NP). The long segment (L segment) expresses the RNA-dependent RNA polymerase (RdRp; L protein) and the matrix protein Z (protein Z), a RING finger protein. The two genes on each segment are separated by a non-coding intergenic region (IGR) and flanked by 5′ and 3′ untranslated regions (UTR). The IGR forms a stable hairpin structure and has been shown to be involved in structure-dependent termination of viral mRNA transcription (Pinschewer et al., 2005, J Virol 79(7): 4519-4526). The terminal nucleotides of the UTR show a high degree of complementarity, thereby thought to result in the formation of secondary structures. These panhandle structures are known to serve as the viral promoter for transcription and replication, and their analysis by site-directed mutagenesis has revealed sequence- and structure-dependence, tolerating not even minor sequence changes (Perez and de la Torre, 2003, Virol 77(2): 1184-1194).

### 2.2 Reverse Genetic System

Isolated and purified RNAs of negative-strand viruses like Pichinde virus cannot directly serve as mRNA i.e., cannot be translated when introduced into cells. Consequently transfection of cells with viral RNA does not lead to production of infectious viral particles. In order to generate infectious viral particles of negative-stranded RNA viruses from cDNA in cultured permissive cells, the viral RNA segment(s) must be trans-complemented with the minimal factors required for transcription and replication. With the help of a minigenome system which has been published several years ago, viral cis-acting elements and transacting factors involved in transcription, replication and formation of viral particles could finally be analyzed (Lee et al., 2000, J Virol 74(8): 3470-3477; Lee et al., 2002, J Virol 76(12): 6393-6397; Perez and de la Torre 2003, J Virol 77(2): 1184-1194; Pinschewer et al., 2003, J Virol 77(6): 3882-3887; Pinschewer et al., 2005, J Virol 79(7): 4519-4526.). Such reverse genetics systems have been developed to successfully demonstrate Pichinde virus rescue (See, eg, Liang et al, 2009, Ann N Y Acad Sci, 1171: E65-E74; Lan et al, 2009, Journal of Virology, 83 (13): 6357-6362).

### 2.3 Recombinant Pichinde Expressing Genes of Interest

The generation of recombinant negative-stranded RNA viruses expressing foreign genes of interest has been pursued for a long time. Different strategies have been published for other viruses (Garcia-Sastre et al., 1994, J Virol 68(10): 6254-6261; Percy et al., 1994, J Virol 68(7): 4486-4492; Flick and Hobom, 1999, Virology 262(1): 93-103; Machado et al., 2003, Virology 313(1): 235-249). Live Pichinde Virus-based vectors have been published (Dhanwani et al., 2015, Journal of Virology 90:2551-2560; International Patent Application Publication No. WO 2016/048949). Tri-segmented Pichinde viruses were published (Dhanwani et al., 2015, Journal of Virology 90:2551-2560; International Patent Application Publication No. WO 2016/048949). In the tri-segmented virus, published by Dhanwani 2015, both NP and GP were kept in their respective natural position in the S segment and thus were expressed under their natural promoters in the flanking UTR.

### 2.4 Replication-Defective Arenavirus

It has been shown that an infectious arenavirus particle can be engineered to contain a genome with the ability to amplify and express its genetic material in infected cells but unable to produce further progeny in normal, not genetically engineered cells (i.e., an infectious, replication-deficient arenavirus particle) (International Publication No.: WO 2009/083210 A1 and International Publication No.: WO 2014/140301 A1).

### 3. SUMMARY OF THE INVENTION

The present application, relates to Pichinde viruses with rearrangements of their ORFs in their genomes. In particular, the present application relates to a Pichinde virus genomic segment that has been engineered to carry a Pichinde virus ORF in a position other than the wild-type position. The present application also provides a tri-segmented Pichinde virus particle comprising one L segment and two S segments or two L segments and one S segment that do not recombine into a replication-competent bi-segmented Pichinde virus particle. The present application demonstrates that the tri-segmented Pichinde virus particle can be engineered to improve genetic stability and ensure lasting transgene expression.

In certain embodiments, a viral vector as provided herein is infectious, i.e., is capable of entering into or injecting its genetic material into a host cell. In certain more specific embodiments, a viral vector as provided herein is infectious, i.e., is capable of entering into or injecting its genetic material into a host cell followed by amplification and expression of its genetic information inside the host cell. In certain embodiments, the viral vector is an infectious, replication-deficient Pichinde virus viral vector engineered to contain a genome with the ability to amplify and express its genetic information in infected cells but unable to produce further infectious progeny particles in normal, not genetically engineered cells. In certain embodiments, the infectious Pichinde virus viral vector is replication-competent and able to produce further infectious progeny particles in normal, not genetically engineered cells. In certain more specific embodiments, such a replication-competent viral vector is attenuated relative to the wild type virus from which the replication-competent viral vector is derived.

### 3.1 Non-Natural Open Reading Frame

Accordingly, in one aspect, provided herein is a Pichinde virus genomic segment. In certain embodiments, the genomic segment is engineered to carry a viral ORF in a position other than the wild-type position of the ORF. In some embodiments, the Pichinde virus genomic segment is selected from the group consisting of:
(i) an S segment, wherein the ORF encoding the NP is under control of a Pichinde virus 5′ UTR;
(ii) an S segment, wherein the ORF encoding the Z protein is under control of a Pichinde virus 5′ UTR;
(iii) an S segment, wherein the ORF encoding the L protein is under control of a Pichinde virus 5′ UTR;
(iv) an S segment, wherein the ORF encoding the GP is under control of a Pichinde virus 3′ UTR;
(v) an S segment, wherein the ORF encoding the L protein is under control of a Pichinde virus 3′ UTR;
(vi) an S segment, wherein the ORF encoding the Z protein is under control of a Pichinde virus 3′ UTR;
(vii) an L segment, wherein the ORF encoding the GP is under control of a Pichinde virus 5′ UTR;
(viii) an L segment, wherein the ORF encoding the NP is under control of a Pichinde virus 5′ UTR;
(ix) an L segment, wherein the ORF encoding the L protein is under control of a Pichinde virus 5′ UTR;
(x) an L segment, wherein the ORF encoding the GP is under control of a Pichinde virus 3′ UTR;
(xi) an L segment, wherein the ORF encoding the NP is under control of a Pichinde virus 3′ UTR; and
(xii) an L segment, wherein the ORF encoding the Z protein is under control of a Pichinde virus 3′ UTR.

In some embodiments, the Pichinde virus 3′ UTR is the 3′ UTR of the Pichinde virus S segment or the Pichinde virus L segment. In certain embodiments, the Pichinde virus 5′ UTR is the 5′ UTR of the Pichinde virus S segment or the Pichinde virus L segment.

Also provided herein is an isolated cDNA of a Pichinde virus genomic segment provided herein. Also provided herein, is a DNA expression vector comprising a cDNA of the Pichinde virus genomic segment.

Also provided herein, is a host cell comprising the Pichinde virus genomic segment, a cDNA of the Pichinde virus genomic segment, or the vector comprising a cDNA of the Pichinde virus genomic segment.

Also provided herein, is a Pichinde virus particle comprising the Pichinde virus genomic segment and a second Pichinde virus genomic segment so that the Pichinde virus particle comprises an S segment and an L segment.

In certain embodiments, the Pichinde virus particle is infectious and replication competent. In some embodiments, the Pichinde virus particle is attenuated. In other embodiments, the Pichinde virus particle is infectious but unable to produce further infectious progeny in non-complementing cells.

In certain embodiments, at least one of the four ORFs encoding GP, NP, Z protein, and L protein is removed or functionally inactivated.

In certain embodiments, at least one of the four ORFs encoding GP, NP, Z protein and L protein is removed and replaced with a heterologous ORF from an organism other than a Pichinde virus. In other embodiments, only one of the four ORFs encoding GP, NP, Z protein and L protein is removed and replaced with a heterologous ORF from an organism other than a Pichinde virus. In a more specific embodiment, the ORF encoding GP is removed and replaced with a heterologous ORF from an organism other than a Pichinde virus. In other embodiments, the ORF encoding NP is removed and replaced with a heterologous ORF from an organism other than a Pichinde virus. In some embodiments, the ORF encoding the Z protein is removed and replaced with a heterologous ORF from an organism other than a Pichinde virus. In other embodiments, the ORF encoding the L protein is removed and replaced with a heterologous ORF from an organism other than a Pichinde virus.

In certain embodiments, the heterologous ORF encodes a reporter protein. In some embodiments, the heterologous ORF encodes an antigen derived from an infectious organism, tumor, or allergen. In other embodiments, the heterologous ORF encoding an antigen is selected from human immunodeficiency virus antigens, hepatitis C virus antigens, hepatitis B surface antigen, varizella zoster virus antigens, cytomegalovirus antigens, mycobacterium tuberculosis antigens, tumor associated antigens, and tumor specific antigens (such as tumor neoantigens and tumor neoepitopes).

In certain embodiments, the growth or infectivity of the Pichinde virus particle is not affected by the heterologous ORF from an organism other than a Pichinde virus.

Also provided herein is a method of producing the Pichinde virus genomic segment. In certain embodiments, the method comprises transcribing the cDNA of the Pichinde virus genomic segment.

Also provided herein is a method of generating the Pichinde virus particle. In certain embodiments the method of generating the Pichinde virus particle comprises:
(i) transfecting into a host cell the cDNA of the Pichinde virus genomic segment;
(ii) transfecting into the host cell a plasmid comprising the cDNA of the second Pichinde virus genomic segment;
(iii) maintaining the host cell under conditions suitable for virus formation; and
(iv) harvesting the Pichinde virus particle.

In certain embodiments, the transcription of the L segment and the S segment is performed using a bidirectional promoter.

In certain embodiments, the method further comprises transfecting into a host cell one or more nucleic acids encoding a Pichinde virus polymerase. In yet more specific embodiments, the polymerase is the L protein. In other embodiments, the method further comprises transfecting into the host cell one or more nucleic acids encoding the NP.

In certain embodiments, transcription of the L segment, and the S segment are each under the control of a promoter selected from the group consisting of:
(i) a RNA polymerase I promoter;
(ii) a RNA polymerase II promoter; and
(iii) a T7 promoter.

In another embodiment, provided herein is a vaccine comprising a Pichinde virus particle, wherein at least one of the four ORFs encoding GP, NP, Z protein, and L protein is removed or functionally inactivated; or wherein at least one ORF encoding GP, NP, Z protein, and L protein is removed and replaced with a heterologous ORF from another organism other than a Pichinde virus; or wherein only one of the four ORFs encoding GP, NP, Z protein, and L protein is removed and replaced with a heterologous ORF from an organism other than a Pichinde virus. In more specific embodiments, the vaccine further comprises a pharmaceutically acceptable carrier.

In another embodiment, provided herein is a pharmaceutical composition comprising a Pichinde virus particle, wherein at least one of the four ORFs encoding GP, NP, Z protein, and L protein is removed or functionally inactivated; or wherein at least one ORF encoding GP, NP, Z protein, and L protein is removed and replaced with a heterologous ORF from another organism other than a Pichinde virus; or wherein only one of the four ORFs encoding GP, NP, Z protein, and L protein is removed and replaced with a heterologous ORF from an organism other than a Pichinde virus. In more specific embodiments, the pharmaceutically acceptable carrier further comprises a pharmaceutically acceptable carrier.

In some embodiments, the Pichinde virus genomic segment or Pichinde virus particle is derived from the highly virulent, high-passaged strain Munchique CoAn4763 isolate P18, or low passaged P2 strain, or is derived from any of the several isolates described by Trapido and colleagues (Trapido et al, 1971, Am J Trop Med Hyg, 20: 631-641).

### 3.2 Tri-Segmented Pichinde Virus

In one aspect, provided herein is a tri-segmented Pichinde virus particle comprising one L segment and two S segments. In some embodiments, propagation of the tri-segmented Pichinde virus particle does not result in a replication-competent bi-segmented viral particle after 70 days of persistent infection in mice lacking type I interferon receptor, type II interferon receptor and recombination activating gene 1 (RAG1), and having been infected with 10⁴ PFU of the tri-segmented Pichinde virus particle. In certain embodiments, inter-segmental recombination of the two S segments, uniting two Pichinde virus ORFs on only one instead of two separate segments, abrogates viral promoter activity.

In another aspect, provided herein is a tri-segmented Pichinde virus particle comprising two L segments and one S segment. In certain embodiments, propagation of the tri-segmented Pichinde virus particle does not result in a replication-competent bi-segmented viral particle after 70 days of persistent infection in mice lacking type I interferon receptor, type II interferon receptor and recombination activating gene 1 (RAG1), and having been infected with 10⁴ PFU of the tri-segmented Pichinde virus particle. In certain embodiments, inter-segmental recombination of the two L segments, uniting two Pichinde virus ORFs on only one instead of two separate segments, abrogates viral promoter activity.

In certain embodiments, one of the two S segments is selected from the group consisting of:
(i) an S segment, wherein the ORF encoding the NP is under control of a Pichinde virus 5′ UTR
(ii) an S segment, wherein the ORF encoding the Z protein is under control of a Pichinde virus 5′ UTR;
(iii) an S segment, wherein the ORF encoding the L protein is under control of a Pichinde virus 5′ UTR;
(iv) an S segment, wherein the ORF encoding the GP is under control of a Pichinde virus 3′ UTR;
(v) an S segment, wherein the ORF encoding the L protein is under control of a Pichinde virus 3′ UTR; and
(vi) an S segment, wherein the ORF encoding the Z protein is under control of a Pichinde virus 3′ UTR.

In certain embodiments, one of the two L segments is selected from the group consisting of:
(i) an L segment, wherein the ORF encoding the GP is under control of a Pichinde virus 5′ UTR;
(ii) an L segment, wherein the ORF encoding the NP is under control of a Pichinde virus 5′ UTR;
(iii) an L segment, wherein the ORF encoding the L protein is under control of a Pichinde virus 5′ UTR;
(iv) an L segment, wherein the ORF encoding the GP is under control of a Pichinde virus 3′ UTR;
(v) an L segment, wherein the ORF encoding the NP is under control of a Pichinde virus 3′ UTR; and
(vi) an L segment, wherein the ORF encoding the Z protein is under control of a Pichinde virus 3′ UTR.

In certain embodiments, the tri-segmented Pichinde virus particle 3′ UTR is the 3′ UTR of the Pichinde virus S segment or the Pichinde virus L segment. In other embodiments, the tri-segmented Pichinde virus particle 5′ UTR is the 5′ UTR of the Pichinde virus S segment or the Pichinde virus L segment.

In certain embodiments, the two S segments comprise (i) one or two heterologous ORFs from an organism other than a Pichinde virus; or (ii) one or two duplicated Pichinde virus ORFs; or (iii) one heterologous ORF from an organism other than a Pichinde virus and one duplicated Pichinde virus ORF.

In certain embodiments, the two L segments comprise (i) one or two heterologous ORFs from an organism other than a Pichinde virus; or (ii) one or two duplicated Pichinde virus ORFs; or (iii) one heterologous ORF from an organism other than a Pichinde virus and one duplicated Pichinde virus ORF.

In certain embodiments, the heterologous ORF encodes an antigen derived from an infectious organism, tumor, or allergen. In other embodiments, the heterologous ORF encoding an antigen is selected from human immunodeficiency virus antigens, hepatitis C virus antigens, hepatitis B surface antigen, varizella zoster virus antigens, cytomegalovirus antigens, mycobacterium tuberculosis antigens, tumor associated antigens, and tumor specific antigens (such as tumor neoantigens and tumor neoepitopes).

In certain embodiments, at least one heterologous ORF encodes a fluorescent protein. In other embodiments the fluorescent protein is a green fluorescent protein (GFP) or red fluorescent protein (RFP).

In certain embodiments, the tri-segmented Pichinde virus particle comprises all four Pichinde virus ORFs. In some embodiments the tri-segmented Pichinde virus particle is infectious and replication competent.

In certain embodiments, the tri-segmented Pichinde virus particle lacks one or more of the four Pichinde virus ORFs. In other embodiments, the tri-segmented Pichinde virus particle is infectious but unable to produce further infectious progeny in non-complementing cells.

In certain embodiments, the tri-segmented Pichinde virus particle lacks one of the four Pichinde virus ORFs, wherein the tri-segmented Pichinde virus particle is infectious but unable to produce further infectious progeny in non-complementing cells.

In some embodiments, the tri-segmented Pichinde virus particle lacks the GP ORF.

In a further aspect, provided herein is a tri-segmented Pichinde virus particle comprising one L segment and two S segments. In certain embodiments, a first S segment is engineered to carry an ORF encoding GP in a position under control of a Pichinde virus 3′ UTR and an ORF encoding a first gene of interest in a position under control of a Pichinde virus 5′ UTR. In some embodiments, a second S segment is engineered to carry an ORF encoding the NP in a position under control of a Pichinde virus 3′ UTR and an ORF encoding a second gene of interest in a position under control of a Pichinde virus 5′ UTR.

In yet another aspect, provided herein, is a tri-segmented Pichinde virus particle comprising one L segment and two S segments. In certain embodiments, a first S segment is engineered to carry an ORF encoding GP in a position under control of a Pichinde virus 5′ UTR and an ORF encoding a first gene of interest in a position under control of a Pichinde virus 3′ UTR. In some embodiments, a second S segment is engineered to carry an ORF encoding NP in a position under control of a Pichinde virus 5′ UTR and an ORF encoding a second gene of interest in a position under control of a Pichinde virus 3′ UTR.

In certain embodiments, the gene of interest encodes an antigen derived from an infectious organism, tumor, or allergen. In other embodiments, the gene of interest encodes an antigen selected from human immunodeficiency virus antigens, hepatitis C virus antigens, hepatitis B surface antigen, varizella zoster virus antigens, cytomegalovirus antigens, mycobacterium tuberculosis antigens, tumor associated antigens, and tumor specific antigens (such as tumor neoantigens and tumor neoepitopes). In yet another embodiment, at least one gene of interest encodes a fluorescent protein. In a specific embodiment, the fluorescent protein is GFP or RFP.

Also provided herein is an isolated cDNA of the genome of the tri-segmented Pichinde virus particle. Also provided herein, is a DNA expression vector comprising a cDNA of the genome of the tri-segmented Pichinde virus particle. Also provided herein is one or more DNA expression vectors comprising either individually or in their totality the cDNA of the tri-segmented Pichinde virus.

Also provided herein, is a host cell comprising the tri-segmented Pichinde virus particle, the cDNA of the genome of the tri-segmented Pichinde virus particle, or the vector comprising the cDNA of the genome of the tri-segmented Pichinde virus particle.

In certain embodiments, the tri-segmented Pichinde virus particle is attenuated.

Also provided herein is a method of generating the tri-segmented Pichinde virus particle. In certain embodiments the method of generating the Pichinde virus particle comprises:
(i) transfecting into a host cell one or more cDNAs of one L segment and two S segments;
(ii) maintaining the host cell under conditions suitable for virus formation; and
(iii) harvesting the Pichinde virus particle.

Also provided herein is a method of generating the tri-segmented Pichinde virus particle. In certain embodiments the method of generating the tri-segmented Pichinde virus particle comprises:
(i) transfecting into a host cell one or more cDNAs of two L segments and one S segment;
(ii) maintaining the host cell under conditions suitable for virus formation; and
(iii) harvesting the Pichinde virus particle.

In certain embodiments, the transcription of the one L segment and two S segment is performed using a bidirectional promoter. In some embodiments, the transcription of the two L segments and one S segment is performed using a bidirectional promoter.

In certain embodiments, the method further comprises transfecting into a host cell one or more nucleic acids encoding a Pichinde virus polymerase. In yet more specific embodiments, the polymerase is the L protein. In other embodiments, the method further comprises transfecting into the host cell one or more nucleic acids encoding the NP protein.

In certain embodiments, transcription of the one L segment, and two S segments are each under the control of a promoter selected from the group consisting of:
(i) a RNA polymerase I promoter;
(ii) a RNA polymerase II promoter; and
(iii) a T7 promoter.

In certain embodiments, transcription of the two L segments, and one S segment are each under the control of a promoter selected from the group consisting of:
(i) a RNA polymerase I promoter;
(ii) a RNA polymerase II promoter; and
(iii) a T7 promoter.

In certain embodiments, the tri-segmented Pichinde virus particle has the same tropism as the bi-segmented Pichinde virus particle. In other embodiments, the tri-segmented Pichinde virus particle is replication deficient.

In another embodiment, provided herein is a vaccine comprising a tri-segmented Pichinde virus particle and a pharmaceutically acceptable carrier.

In another embodiment, provided herein is a pharmaceutical composition comprising a tri-segmented Pichinde virus particle and a pharmaceutically acceptable carrier.

In some embodiments, the Pichinde virus genomic segment or Pichinde virus particle is derived from the highly virulent, high-passaged strain Munchique CoAn4763 isolate P18, or low passaged P2 strain, or is derived from any of the several isolates described by Trapido and colleagues (Trapido et al, 1971, Am J Trop Med Hyg, 20: 631-641).

### 3.3 Conventions and Abbreviations

| Abbreviation | Convention |
|---|---|
| APC | Antigen presenting cell |
| art | Artificial |
| CAT | Chloramphenicol acetyltransferase |
| CMI | cell-mediated immunity |
| CD8 | Cluster of differentiation 8 |
| CD4 | Cluster of differentiation 4 |
| GFP | Green fluorescent protein |
| Abbreviation | Convention |
| GP | Glycoprotein |
| IGR | Intergenic region |
| LCMV | Lymphocytic choriomeningitis virus |
| L protein | RNA-dependent RNA polymerase |
| L segment | Long segment |
| MHC | Major Histocompatibility Complex |
| Z protein | Matrix protein Z |
| NP | Nucleoprotein |
| ORF | Open reading frame |
| RFP | Red fluorescent protein |
| r3PIC | Recombinant tri-segmented Pichinde virus |
| S segment | Short segment |
| UTR | Untranslated region |
| VSV | Vesicular Stomatitis Virus |
| VSVG | Vesicular Stomatitis Virus Glycoprotein |
| GM-CSF | Granulocyte Macrophage Colony-Stimulating |
| | Factor |
| sP1AGM protein | A fusion protein of i) theVSVG signal |
| | peptide, ii) the PIA antigen of the P815 |
| | mouse mastocytoma tumor cell line, iii) a |
| | GSG linker, iv) an enterovirus 2A peptide, |
| | and v) mouse GM-CSF |
| RNP | Ribonucleoprotein |
| RAG1 | Recombination Activating Gene |
| OW | Old World arenaviruses |
| NW | New World arenaviruses |
| LASV | Lassa fever virus |

### 4. BRIEF DESCRIPTION OF THE FIGURES

FIGS. 1A-1D: Schematic representation of the genomic organization of bi- and tri-segmented Pichinde virus. The bi-segmented genome of wild-type Pichinde virus consists of one S segment encoding the GP and NP and one L segment encoding the Z protein and the L protein. Both segments are flanked by the respective 5′ and 3′ UTRs. (FIG. 1A) Schematic description of rPIC^{wt} Pichinde virus genome that was cDNA-derived wild type Pichinde virus with its natural genome segments S (SEQ ID NO: 16) and L (SEQ ID NO: 2), which were modified by silent mutations introduced to abrogate BsmBI and BbsI sites in the respective cDNAs. (FIGS. 1B-1D) The genome of recombinant tri-segmented Pichinde viruses (r3PIC) consists of one L and two S segments with one position where to insert a gene of interest (here GFP/sP1AGM fusion protein) into each one of the S segments. (FIG. 1B) Schematic description of the trisegmented Pichinde virus vector genome with an artificial organization. In one of the duplicated S segments, the glycoprotein (GP) ORF is positioned in lieu of the nucleoprotein (NP) ORF in the natural S segment, i.e. between 3′UTR and IGR. (FIG. 1C) r3PIC-GFP^{art} consists of all viral genes in their natural position, except for the GP ORF, which is artificially juxtaposed to and expressed under control of the 3′ UTR (S-GP/GFPart; SEQ ID NO:13). (FIG. 1D) Schematic description of the trisegmented Pichinde virus-based sP1AGM-expressing r3PIC-sP1AGM^{art} vector genome.

FIG. 2: Trisegmented r3PIC-GFP^{art} was attenuated as compared to its bisegmented wild type parental virus. Growth kinetics of the indicated viruses in BHK-21 cells, infected at a multiplicity of infection (moi) of 0.01 (wild-type Pichinde virus: black squares; r3PIC-GFP^{art}: black circles). Supernatant was taken at the indicated time points after infection and viral titers were determined by focus forming assay.

FIG. 3: Schematic description of the expression cassettes of plasmids used for the experiments described in FIGS. 2 and 4.

FIG. 4: Re-constitution of infectious, GFP-expressing virus from cDNA in cells with r3PIC-GFP^{art}. Fluorescence images of GFP expression captured either 48 or 168 hours after transfection of BHK-21 cells with plasmid combinations as follows:

S segment minigenome: pC-PIC-L-Bsm, pC-PIC-NP-Bbs, pol-I-PIC-miniS-GFP;

L segment minigenome: pC-PIC-L-Bsm, pC-PIC-NP-Bbs, pol-I-PIC-L-GFP-Bsm;

r3PIC-GFP^{art}: pC-PIC-L-Bsm, pC-PIC-NP-Bbs, pol-I-PIC-L, pol-I-PIC-NP-GFP, pol-I-PIC-GP-GFP;

rPIC^{wt}: pC-PIC-L-Bsm, pC-PIC-NP-Bbs, pol-I-PIC-L, pol-I-PIC-S

FIGS. 5A-5B: Trisegmented Pichinde virus based viral vectors are highly immunogenic. BALB/c mice were infected intravenously with 10e5 FFU of r3PIC-sP1AGM^{art}. Control mice were left unimmunized Eight days later, P1A-specific CD8+ T cell frequencies in peripheral blood were determined by MHC class I tetramer staining. Exemplary FACS plots (FIG. 5A) and frequencies of tetramer-binding cells within CD8+ T cell in peripheral blood (FIG. 5B) are shown. Symbols in B represent individual mice.

FIG. 6: Schematic description of the trisegmented Pichinde virus vector genome designed to express its glycoprotein (GP) and nucleoprotein (NP) genes under control of the 5′ and 3′ UTR promoters, respectively, i.e. in their respective “natural” position in the context of an artificially duplicated S segments—S-GP/GFPnat (SEQ ID NO: 15) and S-NP/GFP (also known as PIC-NP-GFP; SEQ ID NO: 11). The genome consists of one L and two S segments with one position where to insert a gene of interest (here GFP protein) into each one of the S segments.

FIG. 7: Early passages of trisegmented r3PIC-GFP^{nat} and r3PIC-GFP^{art} were attenuated as compared to their bisegmented wild type parental virus. Growth kinetics of the indicated viruses in BHK-21 cells in culture, infected at a multiplicity of infection (moi) of 0.01. Supernatant was taken at 48 hours after infection and viral titers were determined by focus forming assay. Symbols show titers from individual parallel cell culture wells; error bars denote the mean+/−SD.

FIG. 8: Unlike r3PIC-GFP^{art}, which is stably attenuated, r3PIC-GFP^{nat} reached titers in the range of rPIC^{wt} during persistent infection of mice. AGR mice (mice triple-deficient in type I and type II interferon receptors as well as RAG1) were infected intravenously with 10e5 FFU of viruses as indicated in the figure (wild-type Pichinde virus—rPIC^{wt}: gray triangles; r3PIC-GFP^{art}: black circles; r3PIC-GFP^{nat}: white squares). Blood was collected on day 7, 14, 21, 28, 35, 42, 56, 77, 98, 120 and 147 and viral infectivity was determined in focus formation assays detecting Pichinde virus nucleoprotein (NP FFU).

FIG. 9: Unlike r3PIC-GFP^{art}, which is stably attenuated, r3PIC-GFP^{nat} reaches titers in the range of rPIC^{wt} during persistent infection of mice. AGR mice (mice triple-deficient in type I and type II interferon receptors as well as RAG1) were infected intravenously with 10e5 FFU of viruses as indicated in the figure (wild-type Pichinde virus—rPIC^{wt}: gray triangles; r3PIC-GFP^{art}: black circles; r3PIC-GFP^{nat}: white squares). Blood was collected on day 7, 14, 21, 28, 35, 42, 56, 77, 98, 120 and 147 and viral infectivity was determined in focus formation assays detecting the viral GFP transgenes in r3PIC-GFP^{nat} and r3PIC-GFP^{art} (GFP FFU).

FIG. 10: Trisegmented Pichinde virus based viral vectors with artificial genomes are highly immunogenic. AGR mice (mice triple-deficient in type I and type II interferon receptors as well as RAG1) were infected intravenously with 10e5 FFU of viruses as indicated in the figure (r3PIC-GFP^{art}: black circles; r3PIC-GFP^{nat}: white squares). Blood was collected on day 7, 14, 21, 28, 35, 42, 56, 77, 98, 120 and 147 and viral infectivity was determined by focus formation assays as displayed in FIG. 9 and FIG. 10. The obtained values were used to calculate the NP: GFP FFU ratio for each animal and time point.

FIG. 11: Virus in mouse serum collected 147 days after r3PIC-GFP^{art} infection showed attenuated growth when directly passaged in cell culture, whereas virus grown from r3PIC-GFP^{nat}-infected mice reached titers comparable to rPIC^{wt}. Serum collected on day 147 after infection on BHK-21 cells was passaged and viral infectivity was determined by NP FFU assays 48 hours later. Symbols show titers of individual mouse serum-derived viruses; error bars denote the mean+/−SD.

FIG. 12: Virus isolated and expanded from mouse serum collected 147 days after r3PIC-GFPart infection showed attenuated growth when directly passaged in cell culture, whereas virus isolated and expanded from r3PIC-GFPnat-infected mice reached titers comparable to rPICwt. BHK-21 cells were infected at a standardized multiplicity of infection=0.01 with viruses that were obtained from serum collected on day 147 after infection and previously passaged for 48 hours. Viral titers were determined 48 hours later. Symbols show titers from individual mouse serum-derived viruses; error bars denote the mean+/−SD

FIG. 13: r3PIC-GFP^{art} failed to recombine its two S segments during a 147 day period of persistent infection in mice, whereas S segment RNA species containing both NP and GP sequences were detected in the serum of mice persistently infected with r3PIC-GFP^{nat} for 147 days. RT-PCR was performed on serum samples collected on day 147 after viral infection, using primers that were designed to bind to Pichinde virus NP and GP, respectively, and that spanned the intergenic region (IGR) of the Pichinde virus S segment such that they were predicted to yield a PCR amplicon of 357 base pairs on the rPIC^{wt} genome template. Each lane represents the RT-PCR product from one individual mouse in the experiment shown in FIGS. 8-10.

### DETAILED DESCRIPTION OF THE INVENTION

4.1 Pichinde Viruses with an Open Reading Frame in a Non-Natural Position

Provided herein are Pichinde viruses with rearrangements of their ORFs. In certain embodiments, such Pichinde viruses are replication competent and infectious. Genomic sequences of such Pichinde viruses are provided herein. In one aspect, provided herein is a Pichinde virus genomic segment, wherein the Pichinde virus genomic segment is engineered to carry a Pichinde virus ORF in a position other than the position in which the respective gene is found in viruses isolated from the wild, such as Pichinde virus strain Munchique CoAn4763 isolate P18 (see SEQ ID NOs: 1 and 2 in 7. Sequence Listing) (referred to herein as “wild-type position”) of the ORF (i.e., a non-natural position).

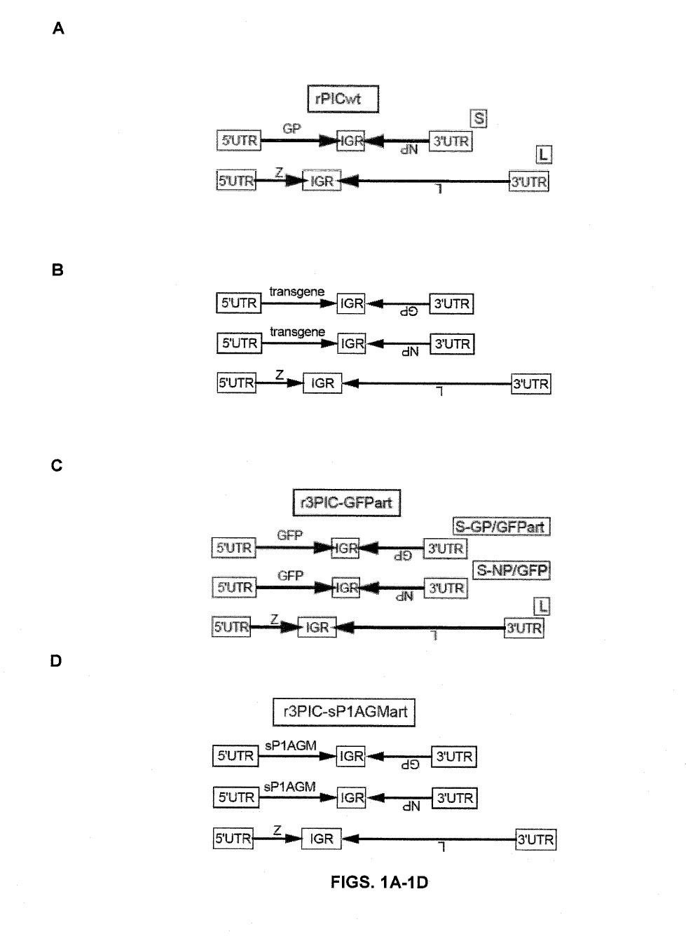

The wild-type Pichinde virus genomic segments and ORFs are known in the art. In particular, the Pichinde virus genome consists of an S segment and an L segment. The S segment carries the ORFs encoding the GP and the NP. The L segment encodes the L protein and the Z protein. Both segments are flanked by the respective 5′ and 3′ UTRs (see FIG. 1A). Illustrative wild-type Pichinde virus genomic segments are provided in SEQ ID NOs: 1 and 2.

In certain embodiments, a Pichinde virus genomic segment can be engineered to carry two or more Pichinde virus ORFs in a position other than the wild-type position. In other embodiments, the Pichinde virus genomic segment can be engineered to carry two Pichinde virus ORFs, or three Pichinde virus ORFs, or four Pichinde virus ORFs in a position other than the wild-type position.

In certain embodiments, a Pichinde virus genomic segment provided herein can be:
(i) a Pichinde virus S segment, wherein the ORF encoding the NP is under control of a Pichinde virus 5′ UTR;
(ii) a Pichinde virus S segment, wherein the ORF encoding the Z protein is under control of a Pichinde virus 5′ UTR;
(iii) a Pichinde virus S segment, wherein the ORF encoding the L protein is under control of a Pichinde virus 5′ UTR;
(iv) a Pichinde virus S segment, wherein the ORF encoding the GP is under control of a Pichinde virus 3′ UTR;
(v) a Pichinde virus S segment, wherein the ORF encoding the L protein is under control of a Pichinde virus 3′ UTR;
(vi) a Pichinde virus S segment, wherein the ORF encoding the Z protein is under control of a Pichinde virus 3′ UTR;
(vii) a Pichinde virus L segment, wherein the ORF encoding the GP is under control of a Pichinde virus 5′ UTR;
(viii) a Pichinde virus L segment, wherein the ORF encoding the NP is under control of a Pichinde virus 5′ UTR;
(ix) a Pichinde virus L segment, wherein the ORF encoding the L protein is under control of a Pichinde virus 5′ UTR;
(x) a Pichinde virus L segment, wherein the ORF encoding the GP is under control of a Pichinde virus 3′ UTR;
(xi) a Pichinde virus L segment, wherein the ORF encoding the NP is under control of a Pichinde virus 3′ UTR; and
(xii) a Pichinde virus L segment, wherein the ORF encoding the Z protein is under control of a Pichinde virus 3′ UTR.

In certain embodiments, the ORF that is in the non-natural position of the Pichinde virus genomic segment described herein can be under the control of a Pichinde virus 3′ UTR or a Pichinde virus 5′ UTR. In more specific embodiments, the Pichinde virus 3′ UTR is the 3′ UTR of the Pichinde virus S segment. In another specific embodiment, the Pichinde virus 3′ UTR is the 3′UTR of the Pichinde virus L segment. In more specific embodiments, the Pichinde virus 5′ UTR is the 5′ UTR of the Pichinde virus S segment. In other specific embodiments, the 5′ UTR is the 5′ UTR of the L segment.

In other embodiments, the ORF that is in the non-natural position of the Pichinde virus genomic segment described herein can be under the control of the arenavirus conserved terminal sequence element (the 5′- and 3′-terminal 19-21-nt regions) (see e.g., Perez & de la Torre, 2003, J Virol. 77(2): 1184-1194).

In certain embodiments, the ORF that is in the non-natural position of the Pichinde virus genomic segment can be under the control of the promoter element of the 5′ UTR (see e.g., Albarino et al., 2011, J Virol., 85(8):4020-4). In another embodiment, the ORF that is in the non-natural position of the Pichinde virus genomic segment can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment or the L segment. In another specific embodiment, the promoter element of the 3′ UTR is the 3′ UTR the promoter element of the S segment or the L segment.

In certain embodiments, the ORF that is in the non-natural position of the Pichinde virus genomic segment can be under the control of a truncated Pichinde virus 3′ UTR or a truncated Pichinde virus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the Pichinde virus S segment or L segment. In more specific embodiments, the truncated 5′ UTR is the 5′ UTR of the Pichinde virus S segment or L segment.

Also provided herein, is a Pichinde virus particle comprising a first genomic segment that has been engineered to carry an ORF in a position other than the wild-type position of the ORF and a second Pichinde virus genomic segment so that the Pichinde virus particle comprises an S segment and an L segment. In specific embodiments, the ORF in a position other than the wild-type position of the ORF is one of the Pichinde virus ORFs.

In certain specific embodiments, the Pichinde virus particle can comprise a full complement of all four Pichinde virus ORFs. In specific embodiments, the second Pichinde virus genomic segment has been engineered to carry an ORF in a position other than the wild-type position of the ORF. In another specific embodiment, the second Pichinde virus genomic segment can be the wild-type genomic segment (i.e., comprises the ORFs on the segment in the wild-type position).

In certain embodiments, the first Pichinde virus genomic segment is an L segment and the second Pichinde virus genomic segment is an S segment. In other embodiments, the first Pichinde virus genomic segment is an S segment and the second Pichinde virus genomic segment is an L segment.

Non-limiting examples of the Pichinde virus particle comprising a genomic segment with an ORF in a position other than the wild-type position of the ORF and a second genomic segment are illustrated in Table 1.

TABLE 1
Pichinde virus particle

| Position 1 | Position 2 | Position 3 | Position 4 |
|---|---|---|---|
| GP | NP | L | Z |
| GP | Z | L | NP |
| GP | Z | NP | L |
| GP | L | NP | Z |
| GP | L | Z | NP |
| NP | GP | L | Z |
| NP | GP | Z | L |
| NP | L | GP | Z |
| NP | L | Z | GP |
| NP | Z | GP | L |
| NP | Z | L | GP |
| Z | GP | L | NP |
| Z | GP | NP | L |
| Z | NP | GP | L |
| Z | NP | L | GP |
| Z | L | NP | GP |
| Z | L | GP | NP |
| L | NP | GP | Z |
| L | NP | Z | GP |
| L | GP | Z | NP |
| L | GP | NP | Z |
| L | Z | NP | GP |
| L | Z | GP | NP |

*Position 1 is under the control of a Pichinde virus S segment 5′ UTR; Position 2 is under the control of a Pichinde virus S segment 3′ UTR; Position 3 is under the control of a Pichinde virus L segment 5′ UTR; Position 4 is under the control of a Pichinde virus L segment 3′ UTR.

Also provided herein, is a cDNA of the Pichinde virus genomic segment engineered to carry an ORF in a position other than the wild-type position of the ORF. In more specific embodiments, provided herein is a cDNA or a set of cDNAs of a Pichinde virus genome as set forth in Table 1.

In certain embodiments, a nucleic acid encoding a Pichinde virsus genome segment described herein can have at least a certain sequence identity to a nucleic acid sequence disclosed herein. Accordingly, in some aspects, a nucleic acid encoding a Pichinde virsus genome segment has a nucleic acid sequence of at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid sequence disclosed herein by SEQ ID NO or a nucleic acid sequence that hybridizes to a nucleic acid sequence disclosed herein by SEQ ID NO. Hybridization conditions can include highly stringent, moderately stringent, or low stringency hybridization conditions that are well known to one of skill in the art such as those described herein. Similarly, a nucleic acid that can be used in generating a Pichinde virus genome segment as described herein can have a certain percent sequence identity to a nucleic acid disclosed herein by SEQ ID NO or a nucleic acid that hybridizes to a nucleic acid sequence disclosed herein by SEQ ID NO. For example, the nucleic acid that is used to generate a Pichinde virus genome segment can have at least 80% identity, at least 85% identity, at least 90% identity, at least 91% identity, at least 92% identity, at least 93% identity, at least 94% identity, at least 95% identity, at least 96% identity, at least 97% identity, at least 98% identity or at least 99% identity, or be identical, to a nucleic acid sequence described herein.

Sequence identity (also known as homology or similarity) refers to sequence similarity between two nucleic acid molecules or between two polypeptides. Identity can be determined by comparing a position in each sequence, which may be aligned for purposes of comparison. When a position in the compared sequence is occupied by the same base or amino acid, then the molecules are identical at that position. A degree of identity between sequences is a function of the number of matching or homologous positions shared by the sequences. The alignment of two sequences to determine their percent sequence identity can be done using software programs known in the art, such as, for example, those described in Ausubel et al., Current Protocols in Molecular Biology, John Wiley and Sons, Baltimore, Md. (1999). Preferably, default parameters are used for the alignment. One alignment program well known in the art that can be used is BLAST set to default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the National Center for Biotechnology Information.

Stringent hybridization refers to conditions under which hybridized polynucleotides are stable. As known to those of skill in the art, the stability of hybridized polynucleotides is reflected in the melting temperature (Tₘ) of the hybrids. In general, the stability of hybridized polynucleotides is a function of the salt concentration, for example, the sodium ion concentration and temperature. A hybridization reaction can be performed under conditions of lower stringency, followed by washes of varying, but higher, stringency. Reference to hybridization stringency relates to such washing conditions. Highly stringent hybridization includes conditions that permit hybridization of only those nucleic acid sequences that form stable hybridized polynucleotides in 0.018M NaCl at 65° C., for example, if a hybrid is not stable in 0.018M NaCl at 65° C., it will not be stable under high stringency conditions, as contemplated herein. High stringency conditions can be provided, for example, by hybridization in 50% formamide, 5×Denhart's solution, 5×SSPE, 0.2% SDS at 42° C., followed by washing in 0.1×SSPE, and 0.1% SDS at 65° C. Hybridization conditions other than highly stringent hybridization conditions can also be used to describe the nucleic acid sequences disclosed herein. For example, the phrase moderately stringent hybridization refers to conditions equivalent to hybridization in 50% formamide, 5×Denhart's solution, 5×SSPE, 0.2% SDS at 42° C., followed by washing in 0.2×SSPE, 0.2% SDS, at 42° C. The phrase low stringency hybridization refers to conditions equivalent to hybridization in 10% formamide, 5×Denhart's solution, 6×SSPE, 0.2% SDS at 22° C., followed by washing in 1×SSPE, 0.2% SDS, at 37° C. Denhart's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M (EDTA). Other suitable low, moderate and high stringency hybridization buffers and conditions are well known to those of skill in the art and are described, for example, in Sambrook and Russell, Molecular Cloning: A laboratory Manual, 3^{rd} edition, Cold Spring Harbor Laboratory N.Y. (2001); and Ausubel et al., Current Protocols in Molecular Biology, John Wiley and Sons, Baltimore, Md. (1999).

In certain embodiments, a cDNA of the Pichinde virus genomic segment that is engineered to carry an ORF in a position other than the wild-type position of the ORF is part of or incorporated into a DNA expression vector. In a specific embodiment, a cDNA of the Pichinde virus genomic segment that is engineered to carry an ORF in a position other than the wild-type position of the ORF is part of or incorporated into a DNA expression vector that facilitates production of a Pichinde virus genomic segment as described herein. In another embodiment, a cDNA described herein can be incorporated into a plasmid. More detailed description of the cDNAs or nucleic acids and expression systems are provided is Section 4.5.1. Techniques for the production of a cDNA are routine and conventional techniques of molecular biology and DNA manipulation and production. Any cloning technique known to the skilled artesian can be used. Such as techniques are well known and are available to the skilled artesian in laboratory manuals such as, Sambrook and Russell, Molecular Cloning: A laboratory Manual, 3^{rd} edition, Cold Spring Harbor Laboratory N.Y. (2001).

In certain embodiments, the cDNA of the Pichinde virus genomic segment that is engineered to carry an ORF in a position other than the wild-type position of the ORF is introduced (e.g., transfected) into a host cell. Thus, in some embodiments provided herein, is a host cell comprising a cDNA of the Pichinde virus genomic segment that is engineered to carry an ORF in a position other than the wild-type position of the ORF (i.e., a cDNA of the genomic segment). In other embodiments, the cDNA described herein is part of or can be incorporated into a DNA expression vector and introduced into a host cell. Thus, in some embodiments provided herein is a host cell comprising a cDNA described herein that is incorporated into a vector. In other embodiments, the Pichinde virus genomic segment described herein is introduced into a host cell.

In certain embodiments, described herein is a method of producing the Pichinde virus genomic segment, wherein the method comprises transcribing the cDNA of the Pichinde virus genomic segment. In certain embodiments, a viral polymerase protein can be present during transcription of the Pichinde virus genomic segment in vitro or in vivo.

In certain embodiments transcription of the Pichinde virus genomic segment is performed using a bi-directional promoter. In other embodiments, transcription of the Pichinde virus genomic segment is performed using a bi-directional expression cassette (see e.g., Ortiz-Riaño et al., 2013, J Gen Virol., 94(Pt 6): 1175-1188). In more specific embodiments the bi-directional expression cassette comprises both a polymerase I and a polymerase II promoter reading from opposite sides into the two termini of the inserted Pichinde virus genomic segment, respectively. In yet more specific embodiments the bi-directional expression cassette with pol-I and pol-II promoters read from opposite sides into the L segment and S segment

In other embodiments, transcription of the cDNA of the Pichinde virus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

In certain embodiments, the method of producing the Pichinde virus genomic segment can further comprise introducing into a host cell the cDNA of the Pichinde virus genomic segment. In certain embodiments, the method of producing the Pichinde virus genomic segment can further comprise introducing into a host cell the cDNA of the Pichinde virus genomic segment, wherein the host cell expresses all other components for production of the Pichinde virus genomic segment; and purifying the Pichinde virus genomic segment from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 4.5.

In certain embodiments, the Pichinde virus particle as described herein results in an infectious and replication competent Pichinde virus particle. In specific embodiments, the Pichinde virus particle described herein is attenuated. In a particular embodiment, the Pichinde virus particle is attenuated such that the virus remains, at least partially, able to spread and can replicate in vivo, but can only generate low viral loads resulting in subclinical levels of infection that are non-pathogenic. Such attenuated viruses can be used as an immunogenic composition. Provided herein, are immunogenic compositions that comprise a Pichinde virus with an ORF in a non-natural position as described in Section 4.7.

4.1.1 Replication-Defective Pichinde Virus Particle with an Open Reading Frame in a Non-Natural Position

In certain embodiments, provided herein is a Pichinde virus particle in which (i) an ORF is in a position other than the wild-type position of the ORF; and (ii) an ORF encoding GP, NP, Z protein, and L protein has been removed or functionally inactivated such that the resulting virus cannot produce further infectious progeny virus particles. A Pichinde virus particle comprising a genetically modified genome in which one or more ORFs has been deleted or functionally inactivated can be produced in complementing cells (i.e., cells that express the Pichinde virus ORF that has been deleted or functionally inactivated). The genetic material of the resulting Pichinde virus particle can be transferred upon infection of a host cell into the host cell, wherein the genetic material can be expressed and amplified. In addition, the genome of the genetically modified Pichinde virus particle described herein can encode a heterologous ORF from an organism other than a Pichinde virus particle.

In certain embodiments, at least one of the four ORFs encoding GP, NP, Z protein, and L protein is removed and replaced with a heterologous ORF from an organism other than a Pichinde virus. In another embodiment, at least one ORF, at least two ORFs, at least three ORFs, or at least four ORFs encoding GP, NP, Z protein and L protein can be removed and replaced with a heterologous ORF from an organism other than a Pichinde virus. In specific embodiments, only one of the four ORFs encoding GP, NP, Z protein, and L protein is removed and replaced with a heterologous ORF from an organism other than a Pichinde virus particle. In more specific embodiments, the ORF that encodes GP of the Pichinde virus genomic segment is removed. In another specific embodiment, the ORF that encodes the NP of the Pichinde virus genomic segment is removed. In more specific embodiments, the ORF that encodes the Z protein of the Pichinde virus genomic segment is removed. In yet another specific embodiment, the ORF encoding the L protein is removed.

Thus, in certain embodiments, the Pichinde virus particle provided herein comprises a genomic segment that (i) is engineered to carry an ORF in a non-natural position; (ii) an ORF encoding GP, NP, Z protein, or L protein is removed; (iii) the ORF that is removed is replaced with a heterologous ORF from an organism other than a Pichinde virus.

In certain embodiments, the heterologous ORF is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the heterologous ORF is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the heterologous ORF encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the heterologous ORF encodes a polypeptide that does not exceed 2500 amino acids in length. In specific embodiments the heterologous ORF does not contain a stop codon. In certain embodiments, the heterologous ORF is codon-optimized. In certain embodiments the nucleotide composition, nucleotide pair composition or both can be optimized. Techniques for such optimizations are known in the art and can be applied to optimize a heterologous ORF.

Any heterologous ORF from an organism other than a Pichinde virus may be included in a Pichinde virus genomic segment. In one embodiment, the heterologous ORF encodes a reporter protein. More detailed description of reporter proteins are described in Section 4.3. In another embodiment, the heterologous ORF encodes an antigen for an infectious pathogen or an antigen associated with any disease that is capable of eliciting an immune response. In specific embodiments the antigen is derived from an infectious organism, a tumor (i.e., cancer), or an allergen. More detailed description on heterologous ORFs is described in Section 4.3.

In certain embodiments, the growth and infectivity of the Pichinde virus particle is not affected by the heterologous ORF from an organism other than a Pichinde virus.

Techniques known to one skilled in the art may be used to produce a Pichinde virus particle comprising a Pichinde virus genomic segment engineered to carry a Pichinde virus ORF in a position other than the wild-type position. For example, reverse genetics techniques may be used to generate such Pichinde virus particle. In other embodiments, the replication-defective Pichinde virus particle (i.e., the Pichinde virus genomic segment engineered to carry a Pichinde virus ORF in a position other than the wild-type position, wherein an ORF encoding GP, NP, Z protein, L protein, has been deleted) can be produced in a complementing cell.

In certain embodiments, the present application relates to the Pichinde virus particle as described herein suitable for use as a vaccine and methods of using such Pichinde virus particle in a vaccination and treatment or prevention of, for example, infections or cancers. More detailed description of the methods of using the Pichinde virus particle described herein is provided in Section 4.6

In certain embodiments, provided herein is a kit comprising, in one or more containers, one or more cDNAs described herein. In a specific embodiment, a kit comprises, in one or two or more containers a Pichinde virus genomic segment or a Pichinde virus particle as described herein. The kit may further comprise one or more of the following: a host cell suitable for rescue of the Pichinde virus genomic segment or the Pichinde virus particle, reagents suitable for transfecting plasmid cDNA into a host cell, a helper virus, plasmids encoding viral proteins and/or one or more primers specific for an modified Pichinde virus genomic segment or Pichinde virus particle or cDNAs of the same.

In certain embodiments, the present application relates to the Pichinde virus particle as described herein suitable for use as a pharmaceutical composition and methods of using such Pichinde virus particle in a vaccination and treatment or prevention of, for example, infections and cancers. More detailed description of the methods of using the Pichinde virus particle described herein is provided in Section 4.7.

### 4.2 Tri-Segmented Pichinde Virus Particle

Provided herein are tri-segmented Pichinde virus particles with rearrangements of their ORFs. In one aspect, provided herein is a tri-segmented Pichinde virus particle comprising one L segment and two S segments or two L segments and one S segment. In certain embodiments, the tri-segmented Pichinde virus particle does not recombine into a replication competent bi-segmented Pichinde virus particle. More specifically, in certain embodiments, two of the genomic segments (e.g., the two S segments or the two L segments, respectively) cannot recombine in a way to yield a single viral segment that could replace the two parent segments. In specific embodiments, the tri-segmented Pichinde virus particle comprises an ORF in a position other than the wild-type position of the ORF. In yet another specific embodiment, the tri-segmented Pichinde virus particle comprises all four Pichinde virus ORFs. Thus, in certain embodiments, the tri-segmented Pichinde virus particle is replication competent and infectious. In other embodiments, the tri-segmented Pichinde virus particle lacks one of the four Pichinde virus ORFs. Thus, in certain embodiments, the tri-segmented Pichinde virus particle is infectious but unable to produce further infectious progeny in non-complementing cells.

In certain embodiments, the ORF encoding GP, NP, Z protein, or the L protein of the tri-segmented Pichinde virus particle described herein can be under the control of a Pichinde virus 3′ UTR or a Pichinde virus 5′ UTR. In more specific embodiments, the tri-segmented Pichinde virus 3′ UTR is the 3′ UTR of a Pichinde virus S segment(s). In another specific embodiment, the tri-segmented Pichinde virus 3′ UTR is the 3′ UTR of a tri-segmented Pichinde virus L segment(s). In more specific embodiments, the tri-segmented Pichinde virus 5′ UTR is the 5′ UTR of a Pichinde virus S segment(s). In other specific embodiments, the 5′ UTR is the 5′ UTR of the L segment(s).

In other embodiments, the ORF encoding GP, NP, Z protein, or the L protein of tri-segmented Pichinde virus particle described herein can be under the control of the arenavirus conserved terminal sequence element (the 5′- and 3′-terminal 19-21-nt regions) (see e.g., Perez & de la Torre, 2003, J Virol. 77(2): 1184-1194).

In certain embodiments, the ORF encoding GP, NP, Z protein or the L protein of the tri-segmented Pichinde virus particle can be under the control of the promoter element of the 5′ UTR (see e.g., Albarino et al., 2011, J Virol., 85(8):4020-4). In another embodiment, the ORF encoding GP, NP Z protein, L protein of the tri-segmented Pichinde virus particle can be under the control of the promoter element of the 3′ UTR (see e.g., Albarino et al., 2011, J Virol., 85(8):4020-4). In more specific embodiments, the promoter element of the 5′ UTR is the 5′ UTR promoter element of the S segment(s) or the L segment(s). In another specific embodiment, the promoter element of the 3′ UTR is the 3′ UTR the promoter element of the S segment(s) or the L segment(s).

In certain embodiments, the ORF that encoding GP, NP, Z protein or the L protein of the tri-segmented Pichinde virus particle can be under the control of a truncated Pichinde virus 3′ UTR or a truncated Pichinde virus 5′ UTR (see e.g., Perez & de la Torre, 2003, J Virol. 77(2): 1184-1194; Albarino et al., 2011, J Virol., 85(8):4020-4). In more specific embodiments, the truncated 3′ UTR is the 3′ UTR of the Pichinde virus S segment or L segment. In more specific embodiments, the truncated 5′ UTR is the 5′ UTR of the Pichinde virus S segment(s) or L segment(s).

Also provided herein, is a cDNA of the tri-segmented Pichinde virus particle. In more specific embodiments, provided herein is a DNA nucleotide sequence or a set of DNA nucleotide sequences encoding a tri-segmented Pichinde virus particle as set forth in Table 2 or Table 3.

In certain embodiments, a nucleic acid encoding a tri-segmented Pichinde virsus genome segment described herein can have at least a certain sequence identity to a nucleic acid sequence disclosed herein. Accordingly, in some aspects, a nucleic acid encoding a tri-segmented Pichinde virsus genome segment has a nucleic acid sequence of at least 80% identity, at least 85% identity, at least 90% identity, at least 91% identity, at least 92% identity, at least 93% identity, at least 94% identity, at least 95% identity, at least 96% identity, at least 97% identity, at least 98% identity, or at least 99% identity, or is identical, to a nucleic acid sequence disclosed herein by SEQ ID NO or a nucleic acid sequence that hybridizes to a nucleic acid sequence disclosed herein by SEQ ID NO. Hybridization conditions can include highly stringent, moderately stringent, or low stringency hybridization conditions that are well known to one of skill in the art such as those described herein. Similarly, a nucleic acid that can be used in generating a tri-segmented Pichinde virus genome segment as described herein can have a certain percent sequence identity to a nucleic acid disclosed herein by SEQ ID NO or a nucleic acid that hybridizes to a nucleic acid sequence disclosed herein by SEQ ID NO. For example, the nucleic acid that is used to generate a tri-segmented Pichinde virus genome segment can have at least 80% identity, at least 85% identity, at least 90% identity, at least 91% identity, at least 92% identity, at least 93% identity, at least 94% identity, at least 95% identity, at least 96% identity, at least 97% identity, at least 98% identity or at least 99% identity, or be identical, to a nucleic acid sequence described herein.

In certain embodiments, the nucleic acids encoding the tri-segmented Pichinde virus genome are part of or incorporated into one or more DNA expression vectors. In a specific embodiment, nucleic acids encoding the genome of the tri-segmented Pichinde virus particle is part of or incorporated into one or more DNA expression vectors that facilitate production of a tri-segmented Pichinde virus particle as described herein. In another embodiment, a cDNA described herein can be incorporated into a plasmid. More detailed description of the cDNAs and expression systems are provided is Section 4.5.1. Techniques for the production of a cDNA routine and conventional techniques of molecular biology and DNA manipulation and production. Any cloning technique known to the skilled artesian can be used. Such techniques are well known and are available to the skilled artesian in laboratory manuals such as, Sambrook and Russell, Molecular Cloning: A laboratory Manual, 3^{rd} edition, Cold Spring Harbor Laboratory N.Y. (2001).

In certain embodiments, the cDNA of the tri-segmented Pichinde virus is introduced (e.g., transfected) into a host cell. Thus, in some embodiments provided herein, is a host cell comprising a cDNA of the tri-segmented Pichinde virus particle (i.e., a cDNA of the genomic segments of the tri-segmented Pichinde virus particle). In other embodiments, the cDNA described herein that is part of or can be incorporated into a DNA expression vector and introduced into a host cell. Thus, in some embodiments provided herein is a host cell comprising a cDNA described herein that is incorporated into a vector. In other embodiments, the tri-segmented Pichinde virus genomic segments (i.e., the L segment and/or S segment or segments) described herein is introduced into a host cell.

In certain embodiments, described herein is a method of producing the tri-segmented Pichinde virus particle, wherein the method comprises transcribing the cDNA of the tri-segmented Pichinde virus particle. In certain embodiments, a viral polymerase protein can be present during transcription of the tri-segmented Pichinde virus particle in vitro or in vivo. In certain embodiments, transcription of the Pichinde virus genomic segment is performed using a bi-directional promoter.

In other embodiments, transcription of the Pichinde virus genomic segment is performed using a bi-directional expression cassette (see e.g., Ortiz-Riario et al., 2013, J Gen Virol., 94(Pt 6): 1175-1188). In more specific embodiments the bi-directional expression cassette comprises both a polymerase I and a polymerase II promoter reading from opposite sides into the two termini of the inserted Pichinde virus genomic segment, respectively.

In other embodiments, transcription of the cDNA of the Pichinde virus genomic segment described herein comprises a promoter. Specific examples of promoters include an RNA polymerase I promoter, an RNA polymerase II promoter, an RNA polymerase III promoter, a T7 promoter, an SP6 promoter or a T3 promoter.

In certain embodiments, the method of producing the tri-segmented Pichinde virus particle can further comprise introducing into a host cell the cDNA of the tri-segmented Pichinde virus particle. In certain embodiments, the method of producing the tri-segmented Pichinde virus particle can further comprise introducing into a host cell the cDNA of the tri-segmented Pichinde virus particle, wherein the host cell expresses all other components for production of the tri-segmented Pichinde virus particle; and purifying the tri-segmented Pichinde virus particle from the supernatant of the host cell. Such methods are well-known to those skilled in the art.

Provided herein are cell lines, cultures and methods of culturing cells infected with nucleic acids, vectors, and compositions provided herein. More detailed description of nucleic acids, vector systems and cell lines described herein is provided in Section 4.5.

In certain embodiments, the tri-segmented Pichinde virus particle as described herein results in an infectious and replication competent Pichinde virus particle. In specific embodiments, the Pichinde virus particle described herein is attenuated. In a particular embodiment, the tri-segmented Pichinde virus particle is attenuated such that the virus remains, at least partially, replication-competent and can replicate in vivo, but can only generate low viral loads resulting in subclinical levels of infection that are non-pathogenic. Such attenuated viruses can be used as an immunogenic composition.

In certain embodiments, the tri-segmented Pichinde virus particle has the same tropism as the bi-segmented Pichinde virus particle.

Also provided herein is a kit comprising, in one or more containers, one or more cDNAs described herein. In a specific embodiment, a kit comprises, in one or two or more containers a tri-segmented Pichinde virus particle as described herein. The kit may further comprise one or more of the following: a host cell suitable for rescue of the tri-segmented Pichinde virus particle, reagents suitable for transfecting plasmid cDNA into a host cell, a helper virus, plasmids encoding viral proteins and/or one or more oligonucleotide primers specific for a modified Pichinde virus genomic segment or Pichinde virus particle or nucleic acids encoding the same.

Also provided herein are immunogenic compositions that comprise the tri-segmented Pichinde virus particle as described in Section 4.6 and 4.7.

### 4.2.1 Tri-Segmented Pichinde Virus Particle Comprising One L Segment and Two S Segments

In one aspect, provided herein is a tri-segmented Pichinde virus particle comprising one L segment and two S segments. In certain embodiments, propagation of the tri-segmented Pichinde virus particle comprising one L segment and two S segments does not result in a replication-competent bi-segmented viral particle. In specific embodiments, propagation of the tri-segmented Pichinde virus particle comprising one L segment and two S segments does not result in a replication-competent bi-segmented viral particle after at least 10 days, at least 20 days, at least 30 days, at least 40 days, at least 50 days, at least 60 days, at least 70 days, at least 80 days, at least 90 days, or at least 100 days of persistent infection in mice lacking type I interferon receptor, type II interferon receptor and recombination activating gene (RAG1), and having been infected with 10⁴ PFU of the tri-segmented Pichinde virus particle (see Section 4.8.13). In other embodiments, propagation of the tri-segmented Pichinde virus particle comprising one L segment and two S segments does not result in a replication-competent bi-segmented viral particle after at least 10 passages, at least 20 passages, at least 30 passages, at least 40 passages, or at least 50 passages.

In certain embodiments, inter-segmental recombination of the two S segments of the tri-segmented Pichinde virus particle, provided herein, that unities the two arenaviral ORFs on one instead of two separate segments results in a non functional promoter (i.e., a genomic segment of the structure: 5′ UTR-5′ UTR or a 3′ UTR-3′ UTR), wherein each UTR forming one end of the genome is an inverted repeat sequence of the other end of the same genome.

In certain embodiments, the tri-segmented Pichinde virus particle comprising one L segment and two S segments has been engineered to carry a Pichinde virus ORF in a position other than the wild-type position of the ORF. In other embodiments, the tri-segmented Pichinde virus particle comprising one L segment and two S segments has been engineered to carry two Pichinde virus ORFs, or three Pichinde virus ORFs, or four Pichinde virus ORFs, or five Pichinde virus ORFs, or six Pichinde virus ORFs in a position other than the wild-type position. In specific embodiments, the tri-segmented Pichinde virus particle comprising one L segment and two S segments comprises a full complement of all four Pichinde virus ORFs. Thus, in some embodiments, the tri-segmented Pichinde virus particle is an infectious and replication competent tri-segmented Pichinde virus particle. In specific embodiments, the two S segments of the tri-segmented Pichinde virus particle have been engineered to carry one of their ORFs in a position other than the wild-type position. In more specific embodiments, the two S segments comprise a full complement of the S segment ORF's. In certain specific embodiments, the L segment has been engineered to carry an ORF in a position other than the wild-type position or the L segment can be the wild-type genomic segment.

In certain embodiments, one of the two S segments can be:
(i) a Pichinde virus S segment, wherein the ORF encoding the Z protein is under control of a Pichinde virus 5′ UTR;
(ii) a Pichinde virus S segment, wherein the ORF encoding the L protein is under control of a Pichinde virus 5′ UTR;
(iii) a Pichinde virus S segment, wherein the ORF encoding the NP is under control of a Pichinde virus 5′ UTR;
(iv) a Pichinde virus S segment, wherein the ORF encoding the GP is under control of a Pichinde virus 3′ UTR;
(v) a Pichinde virus S segment, wherein the ORF encoding the L is under control of a Pichinde virus 3′ UTR; and
(vi) a Pichinde virus S segment, wherein the ORF encoding the Z protein is under control of a Pichinde virus 3′ UTR.

In certain embodiments, the tri-segmented Pichinde virus particle comprising one L segment and two S segments can comprise a duplicate ORF (i.e., two wild-type S segment ORFs e.g., GP or NP). In specific embodiments, the tri-segmented Pichinde virus particle comprising one L segment and two S segments can comprise one duplicate ORF (e.g., (GP, GP)) or two duplicate ORFs (e.g., (GP, GP) and (NP, NP)).

Table 2A, below, is an illustration of the genome organization of a tri-segmented Pichinde virus particle comprising one L segment and two S segments, wherein intersegmental recombination of the two S segments in the tri-segmented Pichinde virus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2A
Tri-segmented Pichinde virus particle comprising one L segment and two S segments

| Position 1 | Position 2 | Position 3 | Position 4 | Position 5 | Position 6 |
|---|---|---|---|---|---|
| *ORF | GP | *ORF | NP | Z | L |
| *ORF | NP | *ORF | GP | Z | L |
| *ORF | NP | *ORF | GP | L | Z |
| *ORF | NP | *ORF | Z | L | GP |
| *ORF | NP | Z | GP | *ORF | Z |
| *ORF | NP | Z | GP | Z | *ORF |
| *ORF | NP | *ORF | L | Z | GP |
| *ORF | L | *ORF | NP | Z | GP |
| *ORF | L | Z | NP | *ORF | GP |
| *ORF | L | *ORF | GP | Z | NP |
| *ORF | L | Z | GP | *ORF | NP |
| *ORF | Z | L | NP | *ORF | GP |
| *ORF | Z | *ORF | GP | L | NP |
| *ORF | Z | L | GP | *ORF | NP |
| L | GP | *ORF | NP | *ORF | Z |
| L | GP | *ORF | *ORF | Z | NP |
| L | GP | *ORF | Z | *ORF | NP |
| L | *ORF | Z | GP | *ORF | NP |
| L | GP | *ORF | NP | *ORF | Z |
| L | GP | *ORF | Z | *ORF | NP |
| L | GP | Z | NP | *ORF | *ORF |
| L | GP | Z | NP | *ORF | *ORF |
| L | *ORF | Z | NP | *ORF | GP |
| L | NP | *ORF | Z | *ORF | GP |
| L | NP | Z | *ORF | GP | *ORF |
| L | *ORF | Z | *ORF | GP | NP |
| L | NP | Z | GP | *ORF | *ORF |
| L | NP | *ORF | Z | *ORF | GP |
| L | *ORF | Z | NP | *ORF | GP |
| L | Z | *ORF | GP | *ORF | NP |
| L | Z | *ORF | NP | *ORF | GP |
| Z | GP | *ORF | NP | *ORF | L |
| Z | GP | *ORF | *ORF | L | NP |
| Z | GP | *ORF | L | *ORF | NP |
| Z | *ORF | L | GP | *ORF | NP |
| Z | GP | *ORF | NP | *ORF | L |
| Z | GP | *ORF | L | *ORF | NP |
| Z | GP | L | NP | *ORF | *ORF |
| Z | GP | L | NP | *ORF | *ORF |
| Z | *ORF | L | NP | *ORF | GP |
| Z | NP | *ORF | *ORF | L | GP |
| Z | NP | *ORF | GP | *ORF | L |
| Z | NP | *ORF | *ORF | L | GP |
| Z | NP | *ORF | L | *ORF | GP |
| Z | NP | L | GP | *ORF | *ORF |
| Z | *ORF | L | GP | *ORF | NP |
| Z | NP | *ORF | GP | *ORF | L |
| Z | NP | *ORF | L | *ORF | GP |
| Z | *ORF | L | NP | *ORF | GP |
| Z | L | *ORF | GP | *ORF | NP |

Position 1 is under the control of a Pichinde virus S segment 5′ UTR; Position 2 is under the control of a Pichinde virus S segment 3′ UTR; Position 3 is under the control of a Pichinde virus S segment 5′ UTR; Position 4 under the control of a Pichinde virus S segment 3′ UTR; Position 5 is under the control of a Pichinde virus L segment 5′ UTR; Position 6 is under the control of a Pichinde virus L segment 3′ UTR.
*ORF indicates that a heterologous ORF has been inserted.

In certain embodiments, the IGR between position one and position two can be a Pichinde virus S segment or L segment IGR; the IGR between position two and three can be a Pichinde virus S segment or L segment IGR; and the IGR between the position five and six can be a Pichinde virus L segment IGR. In a specific embodiment, the IGR between position one and position two can be a Pichinde virus S segment IGR; the IGR between position two and three can be a Pichinde virus S segment IGR; and the IGR between the position five and six can be a Pichinde virus L segment IGR. In certain embodiments, other combinations are also possible. For example, a tri-segmented Pichinde virus particle comprising one L segment and two S segments, wherein intersegmental recombination of the two S segments in the tri-segmented Pichinde virus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, intersegmental recombination of an S segment and an L segment in the tri-segmented Pichinde virus particle comprising one L segment and two S segments, restores a functional segment with two viral genes on only one segment instead of two separate segments. In other embodiments, intersegmental recombination of an S segment and an L segment in the tri-segmented Pichinde virus particle comprising one L segment and two S segments does not result in a replication-competent bi-segmented viral particle.

Table 2B, below, is an illustration of the genome organization of a tri-segmented Pichinde virus particle comprising one L segment and two S segments, wherein intersegmental recombination of an S segment and an L segment in the tri-segmented Pichinde virus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 2B
Tri-segmented Pichinde virus particle comprising one L segment and two S segments

| Position 1 | Position 2 | Position 3 | Position 4 | Position 5 | Position 6 |
|---|---|---|---|---|---|
| L | GP | *ORF | NP | Z | *ORF |
| L | GP | Z | *ORF | *ORF | NP |
| L | GP | *ORF | NP | Z | *ORF |
| L | GP | Z | *ORF | *ORF | NP |
| L | NP | *ORF | GP | Z | *ORF |
| L | NP | Z | *ORF | *ORF | GP |
| L | NP | *ORF | GP | Z | *ORF |
| L | NP | Z | *ORF | *ORF | GP |
| Z | GP | *ORF | NP | L | *ORF |
| Z | GP | L | *ORF | *ORF | NP |
| Z | GP | *ORF | NP | L | *ORF |
| Z | NP | L | *ORF | *ORF | GP |
| Z | NP | *ORF | GP | L | *ORF |
| Z | NP | L | *ORF | *ORF | GP |

Position 1 is under the control of a Pichinde virus S segment 5′ UTR; Position 2 is under the control of a Pichinde virus S segment 3′ UTR; Position 3 is under the control of a Pichinde virus S segment 5′ UTR; Position 4 under the control of a Pichinde virus S segment 3′ UTR; Position 5 is under the control of a Pichinde virus L segment 5′ UTR; Position 6 is under the control of a Pichinde virus L segment 3′ UTR.
*ORF indicates that a heterologous ORF has been inserted.

In certain embodiments, the IGR between position one and position two can be a Pichinde virus S segment or L segment IGR; the IGR between position two and three can be a Pichinde virus S segment or L segment IGR; and the IGR between the position five and six can be a Pichinde virus L segment IGR. In a specific embodiment, the IGR between position one and position two can be a Pichinde virus S segment IGR; the IGR between position two and three can be a Pichinde virus S segment IGR; and the IGR between the position five and six can be a Pichinde virus L segment IGR. In certain embodiments, other combinations are also possible. For example, a tri-segmented Pichinde virus particle comprising one L segment and two S segments, wherein intersegmental recombination of the two S segments in the tri-segmented Pichinde virus genome does not result in a replication-competent bi-segmented viral particle and abrogates arenaviral promoter activity (i.e., the resulting recombined S segment is made up of two 5′UTRs instead of a 3′ UTR and a 5′ UTR).

In certain embodiments, one of skill in the art could construct a Pichinde virus genome with an organization as illustrated in Table 2A or 2B and as described herein, and then use an assay as described in Section 4.8 to determine whether the tri-segmented Pichinde virus particle is genetically stable, i.e., does not result in a replication-competent bi-segmented viral particle as discussed herein.

### 4.2.2 Tri-Segmented Pichinde Virus Particle Comprising Two L Segments and One S Segment

In one aspect, provided herein is a tri-segmented Pichinde virus particle comprising two L segments and one S segment. In certain embodiments, propagation of the tri-segmented Pichinde virus particle comprising two L segments and one S segment does not result in a replication-competent bi-segmented viral particle. In specific embodiments, propagation of the tri-segmented Pichinde virus particle comprising two L segments and one S segment does not result in a replication-competent bi-segmented viral particle after at least 10 days, at least 20 days, at least 30 days, at least 40 days, or at least 50 days, at least 60 days, at least 70 days, at least 80 days, at least 90 days, at least 100 days of persistent in mice lacking type I interferon receptor, type II interferon receptor and recombination activating gene (RAG1), and having been infected with 10⁴ PFU of the tri-segmented Pichinde virus particle (see Section 4.8.13). In other embodiments, propagation of the tri-segmented Pichinde virus particle comprising two L segments and one S segment does not result in a replication-competent bi-segmented viral particle after at least 10 passages, 20 passages, 30 passages, 40 passages, or 50 passages.

In certain embodiments, inter-segmental recombination of the two L segments of the tri-segmented Pichinde virus particle, provided herein, that unities the two Pichinde virus ORFs on one instead of two separate segments results in a non functional promoter (i.e., a genomic segment of the structure: 5′ UTR-5′ UTR or a 3′ UTR-3′ UTR), wherein each UTR forming one end of the genome is an inverted repeat sequence of the other end of the same genome.

In certain embodiments, the tri-segmented Pichinde virus particle comprising two L segments and one S segment has been engineered to carry a Pichinde virus ORF in a position other than the wild-type position of the ORF. In other embodiments, the tri-segmented Pichinde virus particle comprising two L segments and one S segment has been engineered to carry two Pichinde virus ORFs, or three Pichinde virus ORFs, or four Pichinde virus ORFs, or five Pichinde virus ORFs, or six Pichinde virus ORFs in a position other than the wild-type position. In specific embodiments, the tri-segmented Pichinde virus particle comprising two L segments and one S segment comprises a full complement of all four Pichinde virus ORFs. Thus, in some embodiments, the tri-segmented Pichinde virus particle is an infectious and replication competent tri-segmented Pichinde virus particle. In specific embodiments, the two L segments of the tri-segmented Pichinde virus particle have been engineered to carry one of their ORFs in a position other than the wild-type position. In more specific embodiments, the two L segments comprise a full complement of the L segment ORF's. In certain specific embodiments, the S segment has been engineered to carry one of their ORFs in a position other than the wild-type position or the S segment can be the wild-type genomic segment.

In certain embodiments, one of the two L segments can be:
(i) an L segment, wherein the ORF encoding the GP is under control of a Pichinde virus 5′ UTR;
(ii) an L segment, wherein the ORF encoding NP is under control of a Pichinde virus 5′ UTR;
(iii) an L segment, wherein the ORF encoding the L protein is under control of a Pichinde virus 5′ UTR;
(iv) an L segment, wherein the ORF encoding the GP is under control of a Pichinde virus 3′ UTR;
(v) an L segment, wherein the ORF encoding the NP is under control of a Pichinde virus 3′ UTR; and
(vi) an L segment, wherein the ORF encoding the Z protein is under control of a Pichinde virus 3′ UTR.

In certain embodiments, the tri-segmented Pichinde virus particle comprising one L segment and two S segments can comprise a duplicate ORF (i.e., two wild-type L segment ORFs e.g., Z protein or L protein). In specific embodiments, the tri-segmented Pichinde virus particle comprising two L segments and one S segment can comprise one duplicate ORF (e.g., (Z protein, Z protein)) or two duplicate ORFs (e.g., (Z protein, Z protein) and (L protein, L protein)).

Table 3, below, is an illustration of the genome organization of a tri-segmented Pichinde virus particle comprising two L segments and one S segment, wherein intersegmental recombination of the two L segments in the tri-segmented Pichinde virus genome does not result in a replication-competent bi-segmented viral particle and abrogates arenaviral promoter activity (i.e., the putatively resulting recombinant L segment would be made up of two 3′UTRs or two 5′ UTRs instead of a 3′ UTR and a 5′ UTR). Based on Table 3 similar combinations could be predicted for generating a Pichinde virus particle made up of two 5′ UTRs instead of a 3′ UTR and a 5′ UTR.

TABLE 3
Tri-segmented Pichinde virus particle comprising two L segments and one S segment

| Position 1 | Position 2 | Position 3 | Position 4 | Position 5 | Position 6 |
|---|---|---|---|---|---|
| ORF* | Z | ORF* | L | NP | GP |
| ORF* | Z | ORF* | L | GP | NP |
| ORF* | Z | GP | L | ORF* | NP |
| ORF* | Z | ORF* | GP | NP | L |
| ORF* | Z | GP | ORF* | NP | L |
| ORF* | Z | NP | ORF* | GP | L |
| ORF* | ORF* | NP | Z | GP | L |
| ORF* | Z | GP | NP | ORF* | L |
| ORF* | Z | NP | GP | ORF* | L |
| ORF* | L | ORF* | Z | NP | GP |
| ORF* | L | ORF* | Z | GP | NP |
| ORF* | L | ORF* | GP | NP | Z |
| ORF* | L | GP | Z | ORF* | NP |
| ORF* | L | ORF* | GP | NP | Z |
| ORF* | L | NP | Z | ORF* | GP |
| ORF* | L | GP | NP | ORF* | Z |
| ORF* | L | NP | GP | ORF* | Z |
| ORF* | GP | ORF* | L | NP | Z |
| ORF* | GP | NP | L | ORF* | Z |
| ORF* | GP | ORF* | Z | NP | L |
| ORF* | GP | NP | Z | ORF* | L |
| ORF* | NP | ORF* | L | GP | Z |
| ORF* | NP | GP | L | ORF* | Z |
| ORF* | NP | GP | Z | ORF* | L |
| ORF* | NP | ORF* | Z | GP | L |
| ORF* | L | ORF* | Z | NP | GP |
| ORF* | L | ORF* | Z | GP | NP |
| ORF* | L | ORF* | NP | GP | Z |
| ORF* | L | ORF* | GP | NP | Z |
| ORF* | L | NP | Z | ORF* | GP |
| ORF* | Z | ORF* | GP | NP | L |
| ORF* | Z | GP | L | ORF* | NP |
| ORF* | Z | NP | GP | ORF* | L |
| ORF* | Z | GP | NP | ORF* | L |
| ORF* | GP | ORF* | L | NP | Z |
| ORF* | GP | ORF* | L | Z | NP |
| ORF* | GP | ORF* | Z | GP | L |
| ORF* | GP | NP | L | ORF* | Z |
| GP | L | ORF* | Z | ORF* | NP |
| GP | L | ORF* | NP | ORF* | Z |
| GP | Z | ORF* | L | ORF* | NP |
| GP | Z | ORF* | L | ORF* | NP |
| GP | Z | ORF* | NP | ORF* | L |
| GP | NP | ORF* | Z | ORF* | L |
| NP | L | ORF* | Z | ORF* | GP |
| NP | L | ORF* | GP | ORF* | Z |
| NP | L | ORF* | Z | ORF* | GP |

*Position 1 is under the control of a Pichinde virus L segment 5′ UTR; position 2 is under the control of a Pichinde virus L segment 3′ UTR; position 3 is under the control of a Pichinde virus L segment 5′ UTR; position 4 is under the control of a Pichinde virus L segment 3′ UTR; position 5 is under the control of a Pichinde virus S segment 5′ UTR; position 6 is under the control of a Pichinde virus S segment 3′ UTR.
*ORF indicates that a heterologous ORF has been inserted.

In certain embodiments, the IGR between position one and position two can be a Pichinde virus S segment or L segment IGR; the IGR between position two and three can be a Pichinde virus S segment or L segment IGR; and the IGR between the position five and six can be a Pichinde virus S segment or L segment IGR. In a specific embodiment, the IGR between position one and position two can be a Pichinde virus L segment IGR; the IGR between position two and three can be a Pichinde virus L segment IGR; and the IGR between the position five and six can be a Pichinde virus S segment IGR. In certain embodiments, other combinations are also possible.

In certain embodiments intersegmental recombination of an L segment and an S segment from the tri-segmented Pichinde virus particle comprising two L segments and one S segment restores a functional segment with two viral genes on only one segment instead of two separate segments. In other embodiments, intersegmental recombination of an L segment and an S segment in the tri-segmented Pichinde virus particle comprising two L segments and one S segment does not result in a replication-competent bi-segmented viral particle.

Table 3B, below, is an illustration of the genome organization of a tri-segmented Pichinde virus particle comprising two L segments and one S segment, wherein intersegmental recombination of an L segment and an S segment in the tri-segmented Pichinde virus genome does not result in a replication-competent bi-segmented viral particle and abrogates arenaviral promoter activity (i.e., the resulting recombined S segment is made up of two 3′UTRs instead of a 3′ UTR and a 5′ UTR).

TABLE 3B
Tri-segmented Pichinde virus particle comprising two L segments and one S segment

| Position 1 | Position 2 | Position 3 | Position 4 | Position 5 | Position 6 |
|---|---|---|---|---|---|
| NP | Z | *ORF | GP | L | *ORF |
| NP | Z | GP | *ORF | *ORF | L |
| NP | Z | *ORF | GP | L | *ORF |
| NP | Z | GP | *ORF | *ORF | L |
| NP | L | *ORF | GP | Z | *ORF |
| NP | L | GP | *ORF | *ORF | Z |
| NP | L | *ORF | GP | Z | *ORF |
| NP | L | GP | *ORF | *ORF | Z |
| GP | Z | *ORF | NP | L | *ORF |
| GP | Z | NP | *ORF | *ORF | L |
| GP | Z | *ORF | NP | L | *ORF |
| GP | L | NP | *ORF | *ORF | Z |
| GP | L | *ORF | NP | Z | *ORF |
| GP | L | NP | *ORF | *ORF | Z |

*Position 1 is under the control of a Pichinde virus L segment 5′ UTR; position 2 is under the control of a Pichinde virus L segment 3′ UTR; position 3 is under the control of a Pichinde virus L segment 5′ UTR; position 4 is under the control of a Pichinde virus L segment 3′ UTR; position 5 is under the control of a Pichinde virus S segment 5′ UTR; position 6 is under the control of a Pichinde virus S segment 3′ UTR.
*ORF indicates that a heterologous ORF has been inserted.

In certain embodiments, the IGR between position one and position two can be a Pichinde virus S segment or L segment IGR; the IGR between position two and three can be a Pichinde virus S segment or L segment IGR; and the IGR between the position five and six can be a Pichinde virus S segment or L segment IGR. In a specific embodiment, the IGR between position one and position two can be a Pichinde virus L segment IGR; the IGR between position two and three can be a Pichinde virus L segment IGR; and the IGR between the position five and six can be a Pichinde virus S segment IGR. In certain embodiments, other combinations are also possible.

In certain embodiments, one of skill in the art could construct a Pichinde virus genome with an organization as illustrated in Table 3A or 3B and as described herein, and then use an assay as described in Section 4.8 to determine whether the tri-segmented Pichinde virus particle is genetically stable, i.e., does not result in a replication-competent bi-segmented viral particle as discussed herein.

### 4.2.3 Replication-Defective Tri-Segmented Pichinde Virus Particle

In certain embodiments, provided herein is a tri-segmented Pichinde virus particle in which (i) an ORF is in a position other than the wild-type position of the ORF; and (ii) an ORF encoding GP, NP, Z protein, or L protein has been removed or functionally inactivated such that the resulting virus cannot produce further infectious progeny virus particles (i.e., is replication defective). In certain embodiments, the third Pichinde virus segment can be an S segment. In other embodiments, the third Pichinde virus segment can be an L segment. In more specific embodiments, the third Pichinde virus segment can be engineered to carry an ORF in a position other than the wild-type position of the ORF or the third Pichinde virus segment can be the wild-type Pichinde virus genomic segment. In yet more specific embodiments, the third Pichinde virus segment lacks a Pichinde virus ORF encoding GP, NP, Z protein, or the L protein.

In certain embodiments, a tri-segmented genomic segment could be a S or a L segment hybrid (i.e., a genomic segment that can be a combination of the S segment and the L segment). In other embodiments, the hybrid segment is an S segment comprising an L segment IGR. In another embodiment, the hybrid segment is an L segment comprising an S segment IGR. In other embodiments, the hybrid segment is an S segment UTR with and L segment IGR. In another embodiment, the hybrid segment is an L segment UTR with an S segment IGR. In specific embodiments, the hybrid segment is an S segment 5′ UTR with an L segment IGR or an S segment 3′ UTR with an L segment IGR. In other specific embodiments, the hybrid segment is an L segment 5′ UTR with an S segment IGR or an L segment 3′ UTR with an S segment IGR.

A tri-segmented Pichinde virus particle comprising a genetically modified genome in which one or more ORFs has been deleted or functionally inactivated can be produced in complementing cells (i.e., cells that express the Pichinde virus ORF that has been deleted or functionally inactivated). The genetic material of the resulting Pichinde virus particle can be transferred upon infection of a host cell into the host cell, wherein the genetic material can be expressed and amplified. In addition, the genome of the genetically modified Pichinde virus particle described herein can encode a heterologous ORF from an organism other than a Pichinde virus particle.

In certain embodiments, at least one of the four ORFs encoding GP, NP, Z protein, and L protein is removed and replaced with a heterologous ORF from an organism other than a Pichinde virus. In another embodiment, at least one ORF, at least two ORFs, at least three ORFs, or at least four ORFs encoding GP, NP, Z protein and L protein can be removed and replaced with a heterologous ORF from an organism other than a Pichinde virus. In specific embodiments, only one of the four ORFs encoding GP, NP, Z protein, and L protein is removed and replaced with a heterologous ORF from an organism other than a Pichinde virus particle. In more specific embodiments, the ORF that encodes GP of the Pichinde virus genomic segment is removed. In another specific embodiment, the ORF that encodes the NP of the Pichinde virus genomic segment is removed. In more specific embodiments, the ORF that encodes the Z protein of the Pichinde virus genomic segment is removed. In yet another specific embodiment, the ORF encoding the L protein is removed.

In certain embodiments, provided herein is a tri-segmented Pichinde virus particle comprising one L segment and two S segments in which (i) an ORF is in a position other than the wild-type position of the ORF; and (ii) an ORF encoding GP or NP has been removed or functionally inactivated, such that the resulting virus is replication-defective and not infectious. In a specific embodiment, one ORF is removed and replaced with a heterologous ORF from an organism other than a Pichinde virus. In another specific embodiment, two ORFs are removed and replaced with a heterologous ORF from an organism other than a Pichinde virus. In other specific embodiments, three ORFs are removed and replaced with a heterologous ORF from an organism other than a Pichinde virus. In specific embodiments, the ORF encoding GP is removed and replaced with a heterologous ORF from an organism other than a Pichinde virus. In other specific embodiments, the ORF encoding NP is removed and replaced with a heterologous ORF from an organism other than a Pichinde virus. In yet more specific embodiments, the ORF encoding NP and the ORF encoding GP are removed and replaced with one or two heterologous ORFs from an organism other than a Pichinde virus particle. Thus, in certain embodiments the tri-segmented Pichinde virus particle comprises (i) one L segment and two S segments; (ii) an ORF in a position other than the wild-type position of the ORF; (iii) one or more heterologous ORFs from an organism other than a Pichinde virus.

In certain embodiments, provided herein is a tri-segmented Pichinde virus particle comprising two L segments and one S segment in which (i) an ORF is in a position other than the wild-type position of the ORF; and (ii) an ORF encoding the Z protein, and/or the L protein has been removed or functionally inactivated, such that the resulting virus replication-defective and not infectious. In a specific embodiment, one ORF is removed and replaced with a heterologous ORF from an organism other than a Pichinde virus. In another specific embodiment, two ORFs are removed and replaced with a heterologous ORF from an organism other than a Pichinde virus. In specific embodiments, the ORF encoding the Z protein is removed and replaced with a heterologous ORF from an organism other than a Pichinde virus. In other specific embodiments, the ORF encoding the L protein is removed and replaced with a heterologous ORF from an organism other than a Pichinde virus. In yet more specific embodiments, the ORF encoding the Z protein and the ORF encoding the L protein is removed and replaced with a heterologous ORF from an organism other than a Pichinde virus particle. Thus, in certain embodiments the tri-segmented Pichinde virus particle comprises (i) two L segments and one S segment; (ii) an ORF in a position other than the wild-type position of the ORF; (iii) a heterologous ORF from an organism other than a Pichinde virus.

Thus, in certain embodiments, the tri-segmented Pichinde virus particle provided herein comprises a tri-segmented Pichinde virus particle (i.e., one L segment and two S segments or two L segments and one S segment) that i) is engineered to carry an ORF in a non-natural position; ii) an ORF encoding GP, NP, Z protein, or L protein is removed); iii) the ORF that is removed is replaced with one or more heterologous ORFs from an organism other than a Pichinde virus.

In certain embodiments, the heterologous ORF is 8 to 100 nucleotides in length, 15 to 100 nucleotides in length, 25 to 100 nucleotides in length, 50 to 200 nucleotide in length, 50 to 400 nucleotide in length, 200 to 500 nucleotide in length, or 400 to 600 nucleotides in length, 500 to 800 nucleotide in length. In other embodiments, the heterologous ORF is 750 to 900 nucleotides in length, 800 to 100 nucleotides in length, 850 to 1000 nucleotides in length, 900 to 1200 nucleotides in length, 1000 to 1200 nucleotides in length, 1000 to 1500 nucleotides or 10 to 1500 nucleotides in length, 1500 to 2000 nucleotides in length, 1700 to 2000 nucleotides in length, 2000 to 2300 nucleotides in length, 2200 to 2500 nucleotides in length, 2500 to 3000 nucleotides in length, 3000 to 3200 nucleotides in length, 3000 to 3500 nucleotides in length, 3200 to 3600 nucleotides in length, 3300 to 3800 nucleotides in length, 4000 nucleotides to 4400 nucleotides in length, 4200 to 4700 nucleotides in length, 4800 to 5000 nucleotides in length, 5000 to 5200 nucleotides in length, 5200 to 5500 nucleotides in length, 5500 to 5800 nucleotides in length, 5800 to 6000 nucleotides in length, 6000 to 6400 nucleotides in length, 6200 to 6800 nucleotides in length, 6600 to 7000 nucleotides in length, 7000 to 7200 nucleotides in lengths, 7200 to 7500 nucleotides in length, or 7500 nucleotides in length. In some embodiments, the heterologous ORF encodes a peptide or polypeptide that is 5 to 10 amino acids in length, 10 to 25 amino acids in length, 25 to 50 amino acids in length, 50 to 100 amino acids in length, 100 to 150 amino acids in length, 150 to 200 amino acids in length, 200 to 250 amino acids in length, 250 to 300 amino acids in length, 300 to 400 amino acids in length, 400 to 500 amino acids in length, 500 to 750 amino acids in length, 750 to 1000 amino acids in length, 1000 to 1250 amino acids in length, 1250 to 1500 amino acids in length, 1500 to 1750 amino acids in length, 1750 to 2000 amino acids in length, 2000 to 2500 amino acids in length, or more than 2500 or more amino acids in length. In some embodiments, the heterologous ORF encodes a polypeptide that does not exceed 2500 amino acids in length. In specific embodiments the heterologous ORF does not contain a stop codon. In certain embodiments, the heterologous ORF is codon-optimized. In certain embodiments the nucleotide composition, nucleotide pair composition or both can be optimized. Techniques for such optimizations are known in the art and can be applied to optimize a heterologous ORF.

Any heterologous ORF from an organism other than a Pichinde virus may be included in the tri-segmented Pichinde virus particle. In one embodiment, the heterologous ORF encodes a reporter protein. More detailed description of reporter proteins are described in Section 4.3. In another embodiment, the heterologous ORF encodes an antigen for an infectious pathogen or an antigen associated with any disease and where the antigen is capable of eliciting an immune response. In specific embodiments the antigen is derived from an infectious organism, a tumor (i.e., cancer), or an allergen. More detailed description on heterologous ORFs is described in Section 4.3

In certain embodiments, the growth and infectivity of the Pichinde virus particle is not affected by the heterologous ORF from an organism other than a Pichinde virus.

Techniques known to one skilled in the art may be used to produce a Pichinde virus particle comprising a Pichinde virus genomic segment engineered to carry a Pichinde virus ORF in a position other than the wild-type position. For example, reverse genetics techniques may be used to generate such Pichinde virus particle. In other embodiments, the replication-defective Pichinde virus particle (i.e., the Pichinde virus genomic segment engineered to carry a Pichinde virus ORF in a position other than the wild-type position, wherein an ORF encoding GP, NP, Z protein, L protein, has been deleted) can be produced in a complementing cell.

In certain embodiments, the present application relates to the Pichinde virus particle as described herein suitable for use as a vaccine and methods of using such Pichinde virus particle in a vaccination and treatment or prevention of, for example, infections and cancers. More detailed description of the methods of using the Pichinde virus particle described herein is provided in Section 4.6.

In certain embodiments, the present application relates to the Pichinde virus particle as described herein suitable for use as a pharmaceutical composition and methods of using such Pichinde virus particle in a vaccination and treatment or prevention of, for example, infections or cancers. More detailed description of the methods of using the Pichinde virus particle described herein is provided in Section 4.6.

### 4.3 Pichinde Virus Particle or Tri-Segmented Pichinde Virus Particle Expressing a Heterologous ORF

In certain embodiments, the Pichinde virus genomic segment, and the respective Pichinde virus particle or tri-segmented Pichinde virus particle can comprise a heterologous ORF. In other embodiments, the Pichinde virus genomic segment and the respective Pichinde virus particle or tri-segmented Pichinde virus particle can comprise a gene of interest. In more specific embodiments, the heterologous ORF or the gene of interest encodes an antigen. In more specific embodiments, the heterologous ORF or the gene or interest encodes a reporter protein or a fluorescent protein.

In certain embodiments, the Pichinde virus genomic segment, the Pichinde virus particle or the tri-segmented Pichinde virus particle can comprise one or more heterologous ORFs or one or more genes of interest. In other embodiments, the Pichinde virus genomic segment, the Pichinde virus particle or the tri-segmented Pichinde virus particle can comprise at least one heterologous ORF, at least two heterologous ORFs, at least three heterologous ORFs, or more heterologous ORFs. In other embodiments, the Pichinde virus particle or the tri-segmented Pichinde virus particle comprises at least one gene of interest, at least two genes of interest, at least three genes of interest, or more genes of interest.

A wide variety of antigens may be expressed by the Pichinde virus genomic segment, Pichinde virus particle or the tri-segmented Pichinde virus particle of the present application. In one embodiment, the heterologous ORF encodes an antigen of an infectious pathogen or an antigen associated with any disease that is capable of eliciting an immune response. In certain embodiments, the heterologous ORF can encode an antigen derived from a virus, a bacterium, a fungus, a parasite, or can be expressed in a tumor or tumor associated disease (i.e., cancer), an autoimmune disease, a degenerative disease, an inherited disease, substance dependency, obesity, or an allergic disease.

In some embodiments, the heterologous ORF encodes a viral antigen. Non-limiting examples of viral antigens include antigens from adenoviridae (e.g., mastadenovirus and aviadenovirus), herpesviridae (e.g., herpes simplex virus 1, herpes simplex virus 2, herpes simplex virus 5, herpes simplex virus 6, Epstein-Barr virus, HHV6-HHV8 and cytomegalovirus), leviviridae (e.g., levivirus, enterobacteria phase MS2, allolevirus), poxyiridae (e.g., chordopoxyirinae, parapoxvirus, avipoxvirus, capripoxvirus, leporiipoxvirus, suipoxvirus, molluscipoxvirus, and entomopoxyirinae), papovaviridae (e.g., polyomavirus and papillomavirus), paramyxoviridae (e.g., paramyxovirus, parainfluenza virus 1, mobillivirus (e.g., measles virus), rubulavirus (e.g., mumps virus), pneumonovirinae (e.g., pneumovirus, human respiratory syncytial virus), human respiratory syncytial virus and metapneumovirus (e.g., avian pneumovirus and human metapneumovirus), picornaviridae (e.g., enterovirus, rhinovirus, hepatovirus (e.g., human hepatitis A virus), cardiovirus, and apthovirus), reoviridae (e.g., orthoreovirus, orbivirus, rotavirus, cypovirus, fijivirus, phytoreovirus, and oryzavirus), retroviridae (e.g., mammalian type B retroviruses, mammalian type C retroviruses, avian type C retroviruses, type D retrovirus group, BLV-HTLV retroviruses, lentivirus (e.g. human immunodeficiency virus (HIV) 1 and HIV-2 (e.g., HIV gp160), spumavirus), flaviviridae (e.g., hepatitis C virus, dengue virus, West Nile virus), hepadnaviridae (e.g., hepatitis B virus), togaviridae (e.g., alphavirus (e.g., sindbis virus) and rubivirus (e.g., rubella virus)), rhabdoviridae (e.g., vesiculovirus, lyssavirus, ephemerovirus, cytorhabdovirus, and necleorhabdovirus), arenaviridae (e.g., arenavirus, lymphocytic choriomeningitis virus, Ippy virus, and lassa virus), and coronaviridae (e.g., coronavirus and torovirus). In a specific embodiment the viral antigen, is HIV gp120, gp41, HIV Nef, RSV F glycoprotein, RSV G glycoprotein, HTLV tax, herpes simplex virus glycoprotein (e.g., gB, gC, gD, and gE) or hepatitis B surface antigen, hepatitis C virus E protein or coronavirus spike protein. In one embodiment, the viral antigen is not an HIV antigen.

In other embodiments, the heterologous ORF encodes a bacterial antigen (e.g., bacterial coat protein). In other embodiments, the heterologous ORF encodes parasitic antigen (e.g., a protozoan antigen). In yet other embodiments, a heterologous nucleotide sequence encodes a fungal antigen.

Non-limiting examples of bacterial antigens include antigens from bacteria of the Aquaspirillum family, Azospirillum family, Azotobacteraceae family, Bacteroidaceae family, Bartonella species, Bdellovibrio family, Campylobacter species, Chlamydia species (e.g., Chlamydia pneumoniae), clostridium, Enterobacteriaceae family (e.g., Citrobacter species, Edwardsiella, Enterobacter aerogenes, Envinia species, Escherichia coli, Hafnia species, Klebsiella species, Morganella species, Proteus vulgaris, Providencia, Salmonella species, Serratia marcescens, and Shigella flexneri), Gardinella family, Haemophilus influenzae, Halobacteriaceae family, Helicobacter family, Legionallaceae family, Listeria species, Methylococcaceae family, mycobacteria (e.g., Mycobacterium tuberculosis), Neisseriaceae family, Oceanospirillum family, Pasteurellaceae family, Pneumococcus species, Pseudomonas species, Rhizobiaceae family, Spirillum family, Spirosomaceae family, Staphylococcus (e.g., methicillin resistant Staphylococcus aureus and Staphylococcus pyrogenes), Streptococcus (e.g., Streptococcus enteritidis, Streptococcus fasciae, and Streptococcus pneumoniae), Vampirovibr Helicobacter family, Yersinia family, Bacillus antracis and Vampirovibrio family.

Non-limiting examples of parasite antigens include antigens from a parasite such as an amoeba, a malarial parasite, Plasmodium, Trypanosoma cruzi. Non-limiting examples of fungal antigens include antigens from fungus of Absidia species (e.g., Absidia corymbifera and Absidia ramosa), Aspergillus species, (e.g., Aspergillus flavus, Aspergillus fumigatus, Aspergillus nidulans, Aspergillus niger, and Aspergillus terreus), Basidiobolus ranarum, Blastomyces dermatitidis, Candida species (e.g., Candida albicans, Candida glabrata, Candida kern, Candida krusei, Candida parapsilosis, Candida pseudotropicalis, Candida quillermondii, Candida rugosa, Candida stellatoidea, and Candida tropicalis), Coccidioides immitis, Conidiobolus species, Cryptococcus neofonns, Cunninghamella species, dermatophytes, Histoplasma capsulatum, Microsporum gypseum, Mucor pusillus, Paracoccidioides brasiliensis, Pseudallescheria boydii, Rhinosporidium seeberi, Pneumocystis carinii, Rhizopus species (e.g., Rhizopus arrhizus, Rhizopus oryzae, and Rhizopus microsporus), Saccharomyces species, Sporothrix schenckii, zygomycetes, and classes such as Zygomycetes, Ascomycetes, the Basidiomycetes, Deuteromycetes, and Oomycetes.

In some embodiments, a heterologous ORF encodes a tumor antigen or tumor associated antigen. In some embodiments, the tumor antigen or tumor associated antigen includes antigens from tumor associated diseases including acute lymphoblastic leukemia, acute myeloid leukemia, adrenocortical carcinoma, childhood adrenocortical carcinoma, AIDS-Related Cancers, Kaposi Sarcoma, anal cancer, appendix cancer, astrocytomas, atypical teratoid/rhabdoid tumor, basal-cell carcinoma, bile duct cancer, extrahepatic (see cholangiocarcinoma), bladder cancer, bone osteosarcoma/malignant fibrous histiocytoma, brainstem glioma, brain cancer, brain tumor, cerebellar astrocytoma, cerebral astrocytoma/malignant glioma brain tumor, ependymoma, medulloblastoma, supratentorial primitive neuroectodermal tumors, visual pathway and hypothalamic glioma, breast cancer, bronchial adenomas/carcinoids, burkitt's lymphoma, carcinoid tumor, carcinoid gastrointestinal tumor, carcinoma of unknown primary, central nervous system lymphoma, primary, cerebellar astrocytoma, cerebral astrocytoma/malignant glioma, cervical cancer, childhood cancers, chronic bronchitis, chronic lymphocytic leukemia, chronic myelogenous leukemia, chronic myeloproliferative disorders, colon cancer, cutaneous T-cell lymphoma, desmoplastic small round cell tumor, emphysema, endometrial cancer, ependymoma, esophageal cancer, ewing's sarcoma in the Ewing family of tumors, extracranial germ cell tumor, extragonadal germ cell tumor, extrahepatic bile duct cancer, intraocular melanoma, retinoblastoma, gallbladder cancer, gastric (stomach) cancer, gastrointestinal carcinoid tumor, gastrointestinal stromal tumor, germ cell tumor: extracranial, extragonadal, or ovarian gestational trophoblastic tumor, glioma of the brain stem, glioma, childhood cerebral astrocytoma, childhood visual pathway and hypothalamic, gastric carcinoid, hairy cell leukemia, head and neck cancer, heart cancer, hepatocellular (liver) cancer, hodgkin lymphoma, hypopharyngeal cancer, hypothalamic and visual pathway glioma, intraocular melanoma, islet cell carcinoma (endocrine pancreas), kaposi sarcoma, kidney cancer (renal cell cancer), laryngeal cancer, acute lymphoblastic lymphoma, acute lymphocytic leukemia, acute myelogenous leukemia, chronic lymphocytic leukemia, chronic myeloid leukemia, lip and oral cavity cancer, liposarcoma, liver cancer (primary), lung cancer, non-small cell, small cell, AIDS-related lymphoma, Burkitt lymphoma, cutaneous T-cell lymphoma, hodgkin lymphoma, non-hodgkin lymphoma, lymphoma, primary central nervous system, macroglobulinemia, Waldenström, male breast cancer, malignant fibrous histiocytoma of bone/osteosarcoma, medulloblastoma, melanoma, intraocular (eye), merkel cell cancer, mesothelioma, adult malignant, mesothelioma, metastatic squamous neck cancer with occult primary, mouth cancer, multiple endocrine neoplasia syndrome, multiple myeloma/plasma cell neoplasm, mycosis fungoides, myelodysplastic syndromes, myelodysplastic/myeloproliferative diseases, myelogenous leukemia, chronic, myeloid leukemia, adult acute, myeloid leukemia, childhood acute, myeloma, multiple (cancer of the bone-marrow), myeloproliferative disorders, chronic, nasal cavity and paranasal sinus cancer, nasopharyngeal carcinoma, neuroblastoma, non-small cell lung cancer, oligodendroglioma, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, islet cell, paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter, transitional cell cancer, retinoblastoma, rhabdomyosarcoma, childhood, salivary gland cancer, sarcoma, Ewing family of tumors, Kaposi sarcoma, soft tissue sarcoma, uterine sarcoma, sezary syndrome, skin cancer (non-melanoma), skin cancer (melanoma), merkel cell skin carcinoma, small cell lung cancer, small intestine cancer, soft tissue sarcoma, squamous cell carcinoma—see skin cancer (non-melanoma), squamous neck cancer with occult primary, metastatic, stomach cancer, supratentorial primitive neuroectodermal tumor, T-Cell lymphoma, cutaneous—see Mycosis Fungoides and Sezary syndrome, testicular cancer, throat cancer, thymoma and thymic carcinoma, thyroid cancer, childhood transitional cell cancer of the renal pelvis and ureter, gestational trophoblastic tumor, unknown primary site, carcinoma of, adult unknown primary site, cancer of childhood, ureter and renal pelvis, transitional cell cancer, rethral cancer, uterine cancer, endometrial uterine sarcoma, bronchial tumor, central nervous system embryonal tumor; childhood chordoma, colorectal cancer, craniopharyngioma, ependymoblastoma, langerhans cell histiocytosis, acute lymphoblastic leukemia, acute myeloid leukemia (adult/childhood), small cell lung cancer, medulloepithelioma, oral cavity cancer, papillomatosis, pineal parenchymal tumors of intermediate differentiation, pituary tumor, respiratory tract carcinoma involving the NUT gene on chromosome 15, spinal cord tumor, thymoma, thyroid cancer, vaginal Cancer; vulvar Cancer, and Wilms Tumor.

Non-limiting examples of tumor or tumor associated antigens include Adipophilin, AIM-2, ALDH1A1, BCLX (L), BING-4, CALCA, CD45, CPSF, cyclin D1, DKK1, ENAH (hMena), EpCAM, EphA3, EZH2, FGF5, glypican-3, G250/MN/CAIX, HER-2/neu, ID01, IGF2B3, IL13Ralpha2, Intestinal carboxyl esterase, alpha-fetoprotein, Kallikrein 4, KIF20A, Lengsin, M-CSF, MCSP, mdm-2, Meloe, MMP-2, MMP-7, MUC1, MUC5AC, p53, PAX5, PBF, PRAME, PSMA, RAGE-1, RGSS, RhoC, RNF43, RU2AS, secernin 1, SOX10, STEAP1, survivinn, Telomerase, VEGF, or WT1, EGF-R, CEA, CD52, gp 100 protein, MELANA/MART1, NY-ESO-1, p53 MAGE1, MAGE3 and CDK4, alpha-actinin-4, ARTC1, BCR-ABL fusion protein (b3a2), B-RAF, CASP-5, CASP-8, beta-catenin, Cdc27, CDK4, CDKN2A, CLPP, COA-1, dek-can fusion protein, EFTUD2, Elongation factor 2, ETV6-AML1 fusion protein, FLT3-ITD, FN1, GPNMB, LDLR-fucosyltransferaseAS fusion protein, NFYC, OGT, OS-9, pml-RARalpha fusion protein, PRDXS, PTPRK, K-ras, N-ras, RBAF600, SIRT2, SNRPD1, SYT-SSX1 or -SSX2 fusion protein, TGF-betaRll, Triosephosphate isomerase, Lengsin, M-CSF, MCSP, or mdm-2.

In some embodiments, the heterologous ORF encodes a respiratory pathogen antigen. In a specific embodiment, the respiratory pathogen is a virus such as RSV, coronavirus, human metapneumovirus, parainfluenza virus, hendra virus, nipah virus, adenovirus, rhinovirus, or PRRSV. Non-limiting examples of respiratory viral antigens include Respiratory Syncytial virus F, G and M2 proteins, Coronavirus (SARS, HuCoV) spike proteins (S), human metapneumovirus fusion proteins, Parainfluenza virus fusion and hemagglutinin proteins (F, HN), Hendra virus (HeV) and Nipah virus (NiV) attachment glycoproteins (G and F), Adenovirus capsid proteins, Rhinovirus proteins, and PRRSV wild type or modified GP5 and M proteins.

In a specific embodiment, the respiratory pathogen is a bacteria such as Bacillus anthracis, mycobacterium tuberculosis, Bordetella pertussis, streptococcus pneumoniae, yersinia pestis, staphylococcus aureus, Francisella tularensis, legionella pneumophila, chlamydia pneumoniae, pseudomonas aeruginosa, neisseria meningitides, and haemophilus influenzae. Non-limiting examples of respiratory bacterial antigens include Bacillus anthracis Protective antigen PA, Mycobacterium tuberculosis mycobacterial antigen 85A and heat shock protein (Hsp65), Bordetella pertussis pertussis toxoid (PT) and filamentous hemagglutinin (FHA), Streptococcus pneumoniae sortase A and surface adhesin A (PsaA), Yersinia pestis F1 and V subunits, and proteins from Staphylococcus aureus, Francisella tularensis, Legionella pneumophila, Chlamydia pneumoniae, Pseudomonas aeruginosa, Neisseria meningitides, and Haemophilus influenzae.

In some embodiments, the heterologous ORF encodes a T-cell epitope. In other embodiments, the heterologous ORF encodes a cytokine or growth factor.

In other embodiments, the heterologous ORF encodes an antigen expressed in an autoimmune disease. In more specific embodiments, the autoimmune disease can be type I diabetes, multiple sclerosis, rheumatoid arthritis, lupus erythmatosus, and psoriasis. Non-limiting examples of autoimmune disease antigens include Ro60, dsDNA, or RNP.

In other embodiments, ORF encodes an antigen expressed in an allergic disease. In more specific embodiments, the allergic disease can include but is not limited to seasonal and perennial rhinoconjunctivitis, asthma, and eczema. Non-limiting examples of allergy antigens include Bet v 1 and Fel d 1.

In other embodiments, the Pichinde virus genomic segment, the Pichinde virus particle or the tri-segmented Pichinde virus particle further comprises a reporter protein. The reporter protein is capable of expression at the same time as the antigen described herein. Ideally, expression is visible in normal light or other wavelengths of light. In certain embodiments, the intensity of the effect created by the reporter protein can be used to directly measure and monitor the Pichinde virus particle or tri-segmented Pichinde virus particle.

Reporter genes would be readily recognized by one of skill in the art. In certain embodiments, the Pichinde virus particle is a fluorescent protein. In other embodiments, the reporter gene is GFP. GFP emits bright green light when exposed to UV or blue like.

Non-limiting examples of reporter proteins include various enzymes, such as, but not to β-galactosidase, chloramphenicol acetyltransferase, neomycin phosphotransferase, luciferase or RFP.

In certain embodiments, the Pichinde virus genomic segment, the Pichinde virus particle or the tri-segmented Pichinde virus particle expressing a heterologous ORF has desirable properties for use as a vector for vaccination (see e.g., Section 4.6). In another embodiment, the Pichinde virus genomic segment, the Pichinde virus particle or the tri-segmented Pichinde virus particle expressing a heterologous ORF is capable of inducing an immune response in a host (e.g., mouse rabbit, goat, donkey, human). In other embodiments, the Pichinde virus genomic segment, the Pichinde virus particle or the tri-segmented Pichinde virus particle expressing a heterologous ORF described herein induces an innate immune response. In other embodiments, the Pichinde virus genomic segment, the Pichinde virus particle or the tri-segmented Pichinde virus particle expressing a heterologous ORF induces an adaptive immune response. In more specific embodiments, the Pichinde virus genomic segment, the Pichinde virus particle or the tri-segmented Pichinde virus particle expressing a heterologous ORF both an innate and adaptive immune response.

In another embodiment, the Pichinde virus genomic segment, the Pichinde virus particle or the tri-segmented Pichinde virus particle expressing a heterologous ORF induces a T cell response. In yet more specific embodiments, the Pichinde virus genomic segment, the Pichinde virus particle or tri-segmented Pichinde virus particle expressing a heterologous ORF induces a CD8+ T cell response. In other embodiments, the Pichinde virus particle carrying a foreign gene of interest induces a potent CD8+ T cell response of high frequency and functionality. In other embodiments, the Pichinde virus genomic segment, the Pichinde virus particle or the tri-segmented Pichinde virus particle expressing an antigen derived from an infectious organism, a cancer, or an allergen induces CD8+ T cells specific to one or multiple epitopes of the corresponding foreign gene of interest.

In certain embodiments, the Pichinde virus genomic segment, the Pichinde virus particle or the tri-segmented Pichinde virus particle expressing a heterologous ORF can induce T helper 1 differentiation, memory formation of CD4+ T cells and/or elicit durable antibody responses. These antibodies can be neutralizing, opsonizing, toxic to tumor cells or have other favorable biological features. In other embodiments, the Pichinde virus genomic segment, the Pichinde virus particle or tri-segmented Pichinde virus particle expressing a heterologous ORF has a strong tropism for dendritic cells and activates them upon infection. This potentiates presentation of the antigen by antigen presenting cells.

In certain embodiments, the Pichinde virus genomic segment, the Pichinde virus particle or the tri-segmented Pichinde virus particle expressing an antigen derived from an infectious organism, a cancer, or an allergen induces low or undetectable neutralizing antibody titers against Pichinde virus and high protective neutralizing antibody responses to the respective foreign transgene. In some embodiments, the Pichinde virus backbone forming the particle or tri-segmented Pichinde virus particle expressing an antigen derived from an infectious organism, a cancer, or an allergen has low capacity for inducing immunity to the Pichinde viral backbone components.

### 4.4 Generation of a Pichinde Virus Particle and a Tri-Segmented Pichinde Virus Particle

Generally, Pichinde virus particles can be recombinantly produced by standard reverse genetic techniques as described for LCMV, another arenavirus (see Flatz et al., 2006, Proc Natl Acad Sci USA 103:4663-4668; Sanchez et al., 2006, Virology 350:370; Ortiz-Riano et al., 2013, J Gen Virol. 94:1175-88, which are incorporated by reference herein). To generate the Pichinde virus particles provided herein, these techniques can be applied as described below. The genome of the viruses can be modified as described in Section 4.1 and Section 4.2, respectively.

### 4.4.1 Non-Natural Position Open Reading Frame

The generation of a Pichinde virus particle comprising a genomic segment that has been engineered to carry a viral ORF in a position other than the wild-type position of the ORF can be recombinantly produced by any reverse genetic techniques known to one skilled in the art.
(i) Infectious and Replication Competent Pichinde virus Particle

In certain embodiments, the method of generating the Pichinde virus particle comprises (i) transfecting into a host cell the cDNA of the first Pichinde virus genomic segment; (ii) transfecting into a host cell the cDNA of the second Pichinde virus genomic segment; (iii) transfecting into a host cell plasmids expressing the Pichinde virus' minimal trans-acting factors NP and L; (iv) maintaining the host cell under conditions suitable for virus formation; and (v) harvesting the Pichinde virus particle. In certain more specific embodiments, the cDNA is comprised in a plasmid.

Once generated from cDNA, Pichinde virus particles (i.e., infectious and replication competent) can be propagated. In certain embodiments, the Pichinde virus particle can be propagated in any host cell that allows the virus to grow to titers that permit the uses of the virus as described herein. In one embodiment, the host cell allows the Pichinde virus particle to grow to titers comparable to those determined for the corresponding wild-type.

In certain embodiments, the Pichinde virus particle may be propagated in host cells. Specific examples of host cells that can be used include BHK-21, HEK 293, VERO or other. In a specific embodiment, the Pichinde virus particle may be propagated in a cell line.

In certain embodiments, the host cells are kept in culture and are transfected with one or more plasmid(s). The plasmid(s) express the Pichinde virus genomic segment(s) to be generated under control of one or more expression cassettes suitable for expression in mammalian cells, e.g., consisting of a polymerase I promoter and terminator.

Plasmids that can be used for the generation of the Pichinde virus particle can include: i) a plasmid encoding the S genomic segment e.g., pol-I S, ii) a plasmid encoding the L genomic segment e.g., pol-I L. In certain embodiments, the plasmid encoding a Pichinde virus polymerase that direct intracellular synthesis of the viral L and S segments can be incorporated into the transfection mixture. For example, a plasmid encoding the L protein and/or a plasmid encoding NP (pC-L and pC-NP, respectively) can be present. The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In certain embodiments, the Pichinde virus genomic segments are under the control of a promoter. Typically, RNA polymerase I-driven expression cassettes, RNA polymerase II-driven cassettes or T7 bacteriophage RNA polymerase driven cassettes can be used. In certain embodiments, the plasmid(s) encoding the Pichinde virus genomic segments can be the same, i.e., the genome sequence and transacting factors can be transcribed by a promoter from one plasmid. Specific examples of promoters include an RNA polymerase I promoter, an RNA polymerase II promoter, an RNA polymerase III promoter, a T7 promoter, an SP6 promoter or a T3 promoter.

In addition, the plasmid(s) can feature a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of a host cell with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

For recovering the Pichinde virus particle described herein, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (Pichinde virus vector preparation) is harvested, aliquoted and stored at 4° C., −20° C., or −80° C., depending on how long the Pichinde virus vector should be stored prior use. The Pichinde virus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous ORF, wherein a plasmid encoding the genomic segment is modified to incorporated a heterologous ORF. The heterologous ORF can be incorporated into the plasmid using restriction enzymes.
(ii) Infectious, Replication-Defective Pichinde virus Particle

Infectious, replication-defective Pichinde virus particles can be rescued as described above. However, once generated from cDNA, the infectious, replication-deficient Pichinde viruses provided herein can be propagated in complementing cells. Complementing cells are cells that provide the functionality that has been eliminated from the replication-deficient Pichinde virus by modification of its genome (e.g., if the ORF encoding the GP protein is deleted or functionally inactivated, a complementing cell does provide the GP protein).

Owing to the removal or functional inactivation of one or more of the ORFs in Pichinde virus vectors (here deletion of the glycoprotein, GP, will be taken as an example), Pichinde virus vectors can be generated and expanded in cells providing in trans the deleted viral gene(s), e.g., the GP in the present example. Such a complementing cell line, henceforth referred to as C-cells, is generated by transfecting a cell line such as BHK-21, HEK 293, VERO or other with one or more plasmid(s) for expression of the viral gene(s) of interest (complementation plasmid, referred to as C-plasmid). The C-plasmid(s) express the viral gene(s) deleted in the Pichinde virus vector to be generated under control of one or more expression cassettes suitable for expression in mammalian cells, e.g., a mammalian polymerase II promoter such as the EF1 alpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids can be of two types: i) two plasmids, referred to as TF-plasmids for expressing intracellularly in C-cells the minimal transacting factors of the Pichinde virus, is derived from e.g., NP and L proteins of Pichinde virus in the present example; and ii) plasmids, referred to as GS-plasmids, for expressing intracellularly in C-cells the Pichinde virus vector genome segments, e.g., the segments with designed modifications. TF-plasmids express the NP and L proteins of the respective Pichinde virus vector under control of an expression cassette suitable for protein expression in mammalian cells, typically e.g., a mammalian polymerase II promoter such as the CMV or EFlalpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, polI and polll promoters from one plasmid.

For recovering of the Pichinde virus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, polI and polII promoters from one plasmid. For this one can exploit any of the commonly used strategies such as calcium-phosphate, liposome-based protocols or electroporation.

3-5 days later: The culture supernatant (Pichinde virus vector preparation) is harvested, aliquoted and stored at 4° C., −20° C. or −80° C. depending on how long the Pichinde virus vector should be stored prior to use. Then the Pichinde virus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of a antigen in a cell culture wherein the cell culture is infected with an infectious, replication-deficient Pichinde virus expressing a antigen. When used for expression of a antigen in cultured cells, the following two procedures can be used:

i) The cell type of interest is infected with the Pichinde virus vector preparation described herein at a multiplicity of infection (MOI) of one or more, e.g., two, three or four, resulting in production of the antigen in all cells already shortly after infection.

ii) Alternatively, a lower MOI can be used and individual cell clones can be selected for their level of virally driven antigen expression. Subsequently individual clones can be expanded infinitely owing to the non-cytolytic nature of Pichinde virus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of antigen using infectious, replication-deficient Pichinde viruses as vectors may be considered.

### 4.4.2 Generation of a Tri-Segmented Pichinde Virus Particle

A tri-segmented Pichinde virus particle can be recombinantly produced by reverse genetic techniques known in the art, for example as described by Emonet et al., 2008, PNAS, 106(9):3473-3478; Popkin et al., 2011, J. Virol., 85 (15):7928-7932; Dhanwani et al., 2015, Journal of Virology, doi:10.1128/JVI.02705-15, which are incorporated by reference herein. The generation of the tri-segmented Pichinde virus particle provided herein can be modified as described in Section 4.2.
(i) Infectious and Replication Competent Tri-segmented Pichinde virus Particle

In certain embodiments, the method of generating the tri-segmented Pichinde virus particle comprises (i) transfecting into a host cell the cDNAs of the one L segment and two S segments or two L segments and one S segment; (ii) transfecting into a host cell plasmids expressing the Pichinde virus' minimal trans-acting factors NP and L; (iii) maintaining the host cell under conditions suitable for virus formation; and (iv) harvesting the Pichinde virus particle.

Once generated from cDNA, the tri-segmented Pichinde virus particle (i.e., infectious and replication competent) can be propagated. In certain embodiments tri-segmented Pichinde virus particle can be propagated in any host cell that allows the virus to grow to titers that permit the uses of the virus as described herein. In one embodiment, the host cell allows the tri-segmented Pichinde virus particle to grow to titers comparable to those determined for the corresponding wild-type.

In certain embodiments, the tri-segmented Pichinde virus particle may be propagated in host cells. Specific examples of host cells that can be used include BHK-21, HEK 293 or other. In a specific embodiment, the tri-segmented Pichinde virus particle may be propagated in a cell line.

In certain embodiments, the host cells are kept in culture and are transfected with one or more plasmid(s). The plasmid(s) express the Pichinde virus genomic segment(s) to be generated under control of one or more expression cassettes suitable for expression in mammalian cells, e.g., consisting of a polymerase I promoter and terminator.

In specific embodiments, the host cells are kept in culture and are transfected with one or more plasmid(s). The plasmid(s) express the viral gene(s) to be generated under control of one or more expression cassettes suitable for expression in mammalian cells, e.g., consisting of a polymerase I promoter and terminator.

Plasmids that can be used for generating the tri-segmented Pichinde virus comprising one L segment and two S segments can include: i) two plasmids each encoding the S genome segment e.g., pol-I-PIC-S, ii) a plasmid encoding the L genome segment e.g., pol-I-PIC-L. Plasmids needed for the tri-segmented Pichinde virus comprising two L segments and one S segments are: i) two plasmids each encoding the L genome segment e.g., pol-I-PIC-L, ii) a plasmid encoding the S genome segment e.g., pol-I-PIC-S.

In certain embodiments, plasmids encoding a Pichinde virus polymerase that direct intracellular synthesis of the viral L and S segments can be incorporated into the transfection mixture. For example, a plasmid encoding the L protein and a plasmid encoding NP (pC-PIC-L and pC-PIC-NP, respectively). The L protein and NP are the minimal trans-acting factors necessary for viral RNA transcription and replication. Alternatively, intracellular synthesis of viral L and S segments, together with NP and L protein can be performed using an expression cassette with pol-I and pol-II promoters reading from opposite sides into the L and S segment cDNAs of two separate plasmids, respectively.

In addition, the plasmid(s) features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Transfection of BHK-21 cells with a plasmid(s) can be performed using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing clones are identified using Western blot or flow cytometry procedures with antibodies directed against the viral protein(s) of interest.

Typically, RNA polymerase I-driven expression cassettes, RNA polymerase II-driven cassettes or T7 bacteriophage RNA polymerase driven cassettes can be used, the latter preferentially with a 3′-terminal ribozyme for processing of the primary transcript to yield the correct end. In certain embodiments, the plasmids encoding the Pichinde virus genomic segments can be the same, i.e., the genome sequence and transacting factors can be transcribed by T7, polI and polII promoters from one plasmid.

For recovering the Pichinde virus the tri-segmented Pichinde virus vector, the following procedures are envisaged. First day: cells, typically 80% confluent in M6-well plates, are transfected with a mixture of the plasmids, as described above. For this one can exploit any commonly used strategies such as calcium-phosphate, liposome-based protocols or electroporation.

3-5 days later: The cultured supernatant (Pichinde virus vector preparation) is harvested, aliquoted and stored at 4° C., −20° C., or −80° C., depending on how long the Pichinde virus vector should be stored prior use. The Pichinde virus vector preparation's infectious titer is assessed by an immunofocus assay. Alternatively, the transfected cells and supernatant may be passaged to a larger vessel (e.g., a T75 tissue culture flask) on day 3-5 after transfection, and culture supernatant is harvested up to five days after passage.

The present application furthermore relates to expression of a heterologous ORF and/or a gene of interest, wherein a plasmid encoding the genomic segment is modified to incorporated a heterologous ORF and/or a gene of interest. The heterologous ORF and/or gene of interest can be incorporated into the plasmid using restriction enzymes.
(ii) Infectious, Replication-Defective Tri-segmented Pichinde virus Particle

Infectious, replication-defective tri-segmented Pichinde virus particles can be rescued as described above. However, once generated from cDNA, the infectious, replication-deficient Pichinde viruses provided herein can be propagated in complementing cells. Complementing cells are cells that provide the functionality that has been eliminated from the replication-deficient Pichinde virus by modification of its genome (e.g., if the ORF encoding the GP protein is deleted or functionally inactivated, a complementing cell does provide the GP protein).

Owing to the removal or functional inactivation of one or more of the ORFs in Pichinde virus vectors (here deletion of the glycoprotein, GP, will be taken as an example), Pichinde virus vectors can be generated and expanded in cells providing in trans the deleted viral gene(s), e.g., the GP in the present example. Such a complementing cell line, henceforth referred to as C-cells, is generated by transfecting a mammalian cell line such as BHK-21, HEK 293, VERO or other (here BHK-21 will be taken as an example) with one or more plasmid(s) for expression of the viral gene(s) of interest (complementation plasmid, referred to as C-plasmid). The C-plasmid(s) express the viral gene(s) deleted in the Pichinde virus vector to be generated under control of one or more expression cassettes suitable for expression in mammalian cells, e.g., a mammalian polymerase II promoter such as the CMV or EFlalpha promoter with a polyadenylation signal. In addition, the complementation plasmid features a mammalian selection marker, e.g., puromycin resistance, under control of an expression cassette suitable for gene expression in mammalian cells, e.g., polymerase II expression cassette as above, or the viral gene transcript(s) are followed by an internal ribosome entry site, such as the one of encephalomyocarditis virus, followed by the mammalian resistance marker. For production in E. coli, the plasmid additionally features a bacterial selection marker, such as an ampicillin resistance cassette.

Cells that can be used, e.g., BHK-21, HEK 293, MC57G or other, are kept in culture and are transfected with the complementation plasmid(s) using any of the commonly used strategies such as calcium-phosphate, liposome-based protocols or electroporation. A few days later the suitable selection agent, e.g., puromycin, is added in titrated concentrations. Surviving clones are isolated and subcloned following standard procedures, and high-expressing C-cell clones are identified using Western blot or flow cytometry procedures with antibodies directed against the viral protein(s) of interest. As an alternative to the use of stably transfected C-cells transient transfection of normal cells can complement the missing viral gene(s) in each of the steps where C-cells will be used below. In addition, a helper virus can be used to provide the missing functionality in trans.

Plasmids of two types can be used: i) two plasmids, referred to as TF-plasmids for expressing intracellularly in C-cells the minimal transacting factors of the Pichinde virus, is derived from e.g., NP and L proteins of Pichinde virus in the present example; and ii) plasmids, referred to as GS-plasmids, for expressing intracellularly in C-cells the Pichinde virus vector genome segments, e.g., the segments with designed modifications. TF-plasmids express the NP and L proteins of the respective Pichinde virus vector under control of an expression cassette suitable for protein expression in mammalian cells, typically e.g., a mammalian polymerase II promoter such as the CMV or EFlalpha promoter, either one of them preferentially in combination with a polyadenylation signal. GS-plasmids express the small (S) and the large (L) genome segments of the vector. Typically, polymerase I-driven expression cassettes or T7 bacteriophage RNA polymerase (T7-) driven expression cassettes can be used, the latter preferentially with a 3′-terminal ribozyme for processing of the primary transcript to yield the correct end. In the case of using a T7-based system, expression of T7 in C-cells must be provided by either including in the recovery process an additional expression plasmid, constructed analogously to TF-plasmids, providing T7, or C-cells are constructed to additionally express T7 in a stable manner. In certain embodiments, TF and GS plasmids can be the same, i.e., the genome sequence and transacting factors can be transcribed by T7, polI and polll promoters from one plasmid.

For recovering of the Pichinde virus vector, the following procedures can be used. First day: C-cells, typically 80% confluent in M6-well plates, are transfected with a mixture of the two TF-plasmids plus the two GS-plasmids. In certain embodiments, the TF and GS plasmids can be the same, i.e., the genome sequence and transacting factors can be transcribed by T7, polI and polII promoters from one plasmid. For this one can exploit any of the commonly used strategies such as calcium-phosphate, liposome-based protocols or electroporation.

3-5 days later: The culture supernatant (Pichinde virus vector preparation) is harvested, aliquoted and stored at 4° C., −20° C. or −80° C. depending on how long the Pichinde virus vector should be stored prior to use. Then the Pichinde virus vector preparation's infectious titer is assessed by an immunofocus assay on C-cells. Alternatively, the transfected cells and supernatant may be passaged to a larger vessel (e.g., a T75 tissue culture flask) on day 3-5 after transfection, and culture supernatant is harvested up to five days after passage.

The invention furthermore relates to expression of an antigen in a cell culture wherein the cell culture is infected with an infectious, replication-deficient tri-segmented Pichinde virus expressing a antigen. When used for expression of a CMV antigen in cultured cells, the following two procedures can be used:

i) The cell type of interest is infected with the Pichinde virus vector preparation described herein at a multiplicity of infection (MOI) of one or more, e.g., two, three or four, resulting in production of the antigen in all cells already shortly after infection.

ii) Alternatively, a lower MOI can be used and individual cell clones can be selected for their level of virally driven antigen expression. Subsequently individual clones can be expanded infinitely owing to the non-cytolytic nature of Pichinde virus vectors. Irrespective of the approach, the antigen can subsequently be collected (and purified) either from the culture supernatant or from the cells themselves, depending on the properties of the antigen produced. However, the invention is not limited to these two strategies, and other ways of driving expression of CMV antigen using infectious, replication-deficient Pichinde viruses as vectors may be considered.

### 4.5 Nucleic Acids, Vector Systems and Cell Lines

In certain embodiments, provided herein are cDNAs comprising or consisting of the Pichinde virus genomic segment or the tri-segmented Pichinde virus particle as described in Section 4.1 and Section 4.2, respectively.

### 4.5.1 Non-Natural Position Open Reading Frame

In one embodiment, provided herein are nucleic acids that encode an Pichinde virus genomic segment as described in Section 4.1. In more specific embodiments, provided herein is a DNA nucleotide sequence or a set of DNA nucleotide sequences as set forth in Table 1. Host cells that comprise such nucleic acids are also provided Section 4.1.

In specific embodiments, provided herein is a cDNA of the Pichinde virus genomic segment engineered to carry an ORF in a position other than the wild-type position of the ORF, wherein the Pichinde virus genomic segment encodes a heterologous ORF as described in Section 4.1.

In one embodiment, provided herein is a DNA expression vector system that encodes the Pichinde virus genomic segment engineered to carry an ORF in a position other than the wild-type position of the ORF. Specifically, provided herein is a DNA expression vector system wherein one or more vectors encodes two Pichinde virus genomic segments, namely, an L segment and an S segment, of an Pichinde virus particle described herein. Such a vector system can encode (one or more separate DNA molecules).

In another embodiment, provided herein is a cDNA of the Pichinde virus S segment that has been engineered to carry an ORF in a position other than the wild-type position is part of or incorporated into a DNA expression system. In other embodiments, a cDNA of the Pichinde virus L segment that has been engineered to carry an ORF in a position other than the wild-type position is part of or incorporated into a DNA expression system. In certain embodiments, is a cDNA of the Pichinde virus genomic segment that has been engineered to carry (i) an ORF in a position other than the wild-type position of the ORF; and (ii) and ORF encoding GP, NP, Z protein, or L protein has been removed and replaced with a heterologous ORF from an organism other than an Pichinde virus.

In certain embodiments, the cDNA provided herein can be derived from a particular strain of Pichinde virus. Strains of Pichinde virus include Munchique CoAn4763 isolate P18 and their derivatives, P2 and their derivatives, or is derived from any of the several isolates described by Trapido and colleagues (Trapido et al, 1971, Am J Trop Med Hyg, 20: 631-641). In specific embodiments, the cDNA is derived from Pichinde virus Munchique CoAn4763 isolate P18 strain.

In certain embodiments, the vector generated to encode an Pichinde virus particle or a tri-segmented Pichinde virus particle as described herein may be based on a specific strain of Pichinde virus. Strains of Pichinde virus include Munchique CoAn4763 isolate P18 and their derivatives, P2 and their derivatives, or is derived from any of the several isolates described by Trapido and colleagues (Trapido et al, 1971, Am J Trop Med Hyg, 20: 631-641). In certain embodiments, an Pichinde virus particle or a tri-segmented Pichinde virus particle as described herein may be based on Pichinde virus Munchique CoAn4763 isolate P18 strain. The sequence of the S segment of Pichinde virus strain Munchique CoAn4763 isolate P18 is listed as SEQ ID NO: 1. In certain embodiments, the sequence of the S segment of Pichinde virus strain Munchique CoAn4763 isolate P18 is the sequence set forth in SEQ ID NO: 1. The sequence of the L segment of Pichinde virus is listed as SEQ ID NO: 2.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the Pichinde virus genomic segment that has been engineered to carry an ORF in a position other than the wild-type position of the ORF. In some embodiments, the cell comprises the S segment and/or the L segment.

### 4.5.2 Tri-Segmented Pichinde Virus Particle

In one embodiment, provided herein are nucleic acids that encode a tri-segmented Pichinde virus particle as described in Section 4.2. In more specific embodiments, provided herein is a DNA nucleotide sequence or a set of DNA nucleotide sequences, for example, as set forth in Table 2 or Table 3. Host cells that comprise such nucleic acids are also provided Section 4.2.

In specific embodiments, provided herein is a cDNA consisting of a cDNA of the tri-segmented Pichinde virus particle that has been engineered to carry an ORF in a position other than the wild-type position of the ORF. In other embodiments, is a cDNA of the tri-segmented Pichinde virus particle that has been engineered to (i) carry a Pichinde virus ORF in a position other than the wild-type position of the ORF; and (ii) wherein the tri-segmented Pichinde virus particle encodes a heterologous ORF as described in Section 4.2.

In one embodiment, provided herein is a DNA expression vector system that together encode the tri-segmented Pichinde virus particle as described herein. Specifically, provided herein is a DNA expression vector system wherein one or more vectors encode three Pichinde virus genomic segments, namely, one L segment and two S segments or two L segments and one S segment of a tri-segmented Pichinde virus particle described herein. Such a vector system can encode (one or more separate DNA molecules).

In another embodiment, provided herein is a cDNA of the Pichinde virus S segment(s) that has been engineered to carry an ORF in a position other than the wild-type position, and is part of or incorporated into a DNA expression system. In other embodiments, a cDNA of the Pichinde virus L segment(s) that has been engineered to carry an ORF in a position other than the wild-type position is part of or incorporated into a DNA expression system. In certain embodiments, is a cDNA of the tri-segmented Pichinde virus particle that has been engineered to carry (i) an ORF in a position other than the wild-type position of the ORF; and (ii) an ORF encoding GP, NP, Z protein, or L protein has been removed and replaced with a heterologous ORF from an organism other than a Pichinde virus.

In certain embodiments, the cDNA provided herein can be derived from a particular strain of Pichinde virus. Strains of Pichinde virus include Munchique CoAn4763 isolate P18 and their derivatives, P2 and their derivatives, or is derived from any of the several isolates described by Trapido and colleagues (Trapido et al, 1971, Am J Trop Med Hyg, 20: 631-641). In specific embodiments, the cDNA is derived from Pichinde virus Munchique CoAn4763 isolate P18 strain.

In certain embodiments, the vector generated to encode an Pichinde virus particle or a tri-segmented Pichinde virus particle as described herein may be based on a specific strain of Pichinde virus. Strains of Pichinde virus include Munchique CoAn4763 isolate P18 and their derivatives, P2 and their derivatives, or is derived from any of the several isolates described by Trapido and colleagues (Trapido et al, 1971, Am J Trop Med Hyg, 20: 631-641). In certain embodiments, an Pichinde virus particle or a tri-segmented Pichinde virus particle as described herein may be based on Pichinde virus Munchique CoAn4763 isolate P18 strain. The sequence of the S segment of Pichinde virus strain Munchique CoAn4763 isolate P18 is listed as SEQ ID NO: 1. In certain embodiments, the sequence of the S segment of Pichinde virus strain Munchique CoAn4763 isolate P18 is the sequence set forth in SEQ ID NO: 1. A sequence of the L segment of Pichinde virus is listed as SEQ ID NO: 2.

In another embodiment, provided herein is a cell, wherein the cell comprises a cDNA or a vector system described above in this section. Cell lines derived from such cells, cultures comprising such cells, methods of culturing such cells infected are also provided herein. In certain embodiments, provided herein is a cell, wherein the cell comprises a cDNA of the tri-segmented Pichinde virus particle. In some embodiments, the cell comprises the S segment and/or the L segment.

### 4.6 Methods of Use

Vaccines have been successful for preventing and/or treating infectious diseases, such as those for polio virus and measles. However, therapeutic immunization in the setting of established, chronic disease, including both chronic infections and cancer has been less successful. The ability to generate a Pichinde virus particle and/or a tri-segmented Pichinde virus particle represents a new novel vaccine strategy.

In one embodiment, provided herein are methods of treating an infection and/or cancer in a subject comprising administering to the subject one or more types of Pichinde virus particles or tri-segmented Pichinde virus particles, as described herein or a composition thereof. In a specific embodiment, a method for treating an infection and/or cancer described herein comprises administering to a subject in need thereof an effective amount of one or more Pichinde virus particles or tri-segmented Pichinde virus particles, described herein or a composition thereof. The subject can be a mammal, such as but not limited to a human being, a mouse, a rat, a guinea pig, a domesticated animal, such as, but not limited to, a cow, a horse, a sheep, a pig, a goat, a cat, a dog, a hamster, a donkey. In a specific embodiment, the subject is a human. The human subject might be male, female, adults, children, seniors (65 and older), and those with multiple diseases (i.e., a polymorbid subject). In certain embodiments, subjects are those whose disease has progressed after treatment with chemotherapy, radiotherapy, surgery, and/or biologic agents.

In another embodiment, provided herein are methods for inducing an immune response against an antigen derived from an infectious organism, tumor, or allergen in a subject comprising administering to the subject a Pichinde virus particle or a tri-segmented Pichinde virus particle expressing an antigen derived from an infectious organism, tumor, or allergen or a composition thereof.

In another embodiment, the subjects to whom a Pichinde virus particle or tri-segmented Pichinde virus particle expressing an antigen derived from an infectious organism, tumor, or allergen described herein or a composition thereof is administered have, are susceptible to, or are at risk for a infection, development of cancer or a allergy, or exhibit a pre-cancerous tissue lesion. In another specific embodiment, the subjects to whom a Pichinde virus particle or tri-segmented Pichinde virus particle expressing an antigen derived from an infectious organism, tumor, or allergen described herein or a composition thereof is administered are infected with, are susceptible to, are at risk for, or diagnosed with an infection, cancer, pre-cancerous tissue lesion, or allergy.

In another embodiment, the subjects to whom a Pichinde virus particle or tri-segmented Pichinde virus particle expressing an antigen derived from an infectious organism, tumor, or allergen described herein or a composition thereof is administered are suffering from, are susceptible to, or are at risk for, an infection, a cancer, a pre-cancerous lesion, or an allergy in the pulmonary system, central nervous system, lymphatic system, gastrointestinal system, or circulatory system among others. In a specific embodiment, the subjects to whom a Pichinde virus particle or tri-segmented Pichinde virus particle expressing an antigen derive from an infectious organism, tumor, or allergen described herein or a composition thereof is administered are suffering from, are susceptible to, or are at risk for, an infection, a cancer, or an allergy in one or more organs of the body, including but not limited to the brain, liver, lungs, eyes, ears, intestines, esophagus, uterus, nasopharynx or salivary glands.

In another embodiment, the subjects to whom a Pichinde virus particle or tri-segmented Pichinde virus particle expressing an antigen derived from an infectious organism, a cancer, or an allergen described herein or a composition thereof is administered to a subject suffering from symptoms including but not limited to fever, night sweats, tiredness, malaise, uneasiness, sore throat, swollen glands, joint pain, muscle pain, loss of appetite, weight loss, diarrhea, gastrointestinal ulcerations, gastrointestinal bleeding, shortness of breath, pneumonia, mouth ulcers, vision problems, hepatitis, jaundice, encephalitis, seizures, coma, pruritis, erythema, hyperpigmentation, changes in lymph node, or hearing loss.

In another embodiment, a Pichinde virus or tri-segmented Pichinde virus particle expressing an antigen derived from an infectious organism, a cancer, or an allergen as described herein or a composition thereof is administered to a subject of any age group suffering from, are susceptible to, or are at risk for, an infection, a cancer, or an allergy. In a specific embodiment, a Pichinde virus particle or a tri-segmented Pichinde virus particle expressing an antigen derived from an infectious organism, a cancer, or an allergen as described herein or a composition thereof is administered to a subject with a compromised immune system, a pregnant subject, a subject undergoing an organ or bone marrow transplant, a subject taking immunosuppressive drugs, a subject undergoing hemodialysis, a subject who has cancer, or a subject who is suffering from, are susceptible to, or are at risk for, an infection, a cancer, or an allergy. In a more specific embodiment, a Pichinde virus particle or a tri-segmented Pichinde virus particle expressing an antigen derived from an infectious organism, a cancer, or an allergen as described herein or a composition thereof is administered to a subject who is a child of 0, 1, 2, 3, 4, 5, 6, 7, 8, 9, 10, 11, 12, 13, 14, 15, 16, or 17 years of age suffering from, are susceptible to, or are at risk for, an infection, a cancer, or an allergy. In yet another specific embodiment, a Pichinde virus particle or a tri-segmented Pichinde virus particle expressing an antigen derived from an infectious organism, a cancer, or an allergen described herein or a composition thereof is administered to a subject who is an infant suffering from, is susceptible to, or is at risk for, an infection, cancer or an allergy. In yet another specific embodiment, a Pichinde virus particle or tri-segmented Pichinde virus particle expressing an antigen derived from an infectious organism, a cancer, or an allergen described herein or a composition thereof is administered to a subject who is an infant of 0, 1, 2, 3, 4, 5, 6, 7, 8, 9, 10, 11, or 12 months of age suffering from, is susceptible to, or is at risk for, an infection, cancer, or an allergy. In yet another specific embodiment, a Pichinde virus particle or tri-segmented Pichinde virus particle expressing an antigen derived from an infectious organism, a cancer, or an allergen described herein or a composition thereof is administered to an elderly subject who is suffering from, is susceptible to, or is at risk for, an infection, cancer, or an allergy. In a more specific embodiment, a Pichinde virus particle or a tri-segmented Pichinde virus particle expressing an antigen derived from an infectious organism, a cancer, or an allergen described herein or a composition thereof is administered to a subject who is a senior subject of 65, 66, 67, 68, 69, 70, 71, 72, 73, 74, 75, 76, 77, 78, 79, 80, 81, 82, 83, 84, 85, 86, 87, 88, 89, or 90 years of age.

In another embodiment, a Pichinde virus particle or tri-segmented Pichinde virus particle expressing an antigen derived from an infectious organism, a cancer, or an allergen described herein or a composition thereof is administered to subjects with a heightened risk of disseminated infection, a cancer, or an allergy. In a specific embodiment, Pichinde virus particle or a tri-segmented Pichinde virus particle expressing an antigen derived from an infectious organism, a cancer, or an allergen described herein or a composition thereof is administered to subjects in the neonatal period with a neonatal and therefore immature immune system.

In another embodiment, a Pichinde virus particle or tri-segmented Pichinde virus particle expressing an antigen derived from an infectious organism, a cancer, or an allergen as described herein or a composition thereof is administered to a subject having a dormant infection, cancer, or allergy. In a specific embodiment, a Pichinde virus particle or a tri-segmented Pichinde virus expressing an antigen derived from an infectious organism, a cancer, or an allergen described herein or a composition thereof is administered to a subject having a dormant infection, a dormant cancer, or a dormant allergy which can reactivate upon immune system compromise. Thus, provided herein is a method for preventing reactivation of an infection, a cancer, or an allergy.

In another embodiment, a Pichinde virus particle or tri-segmented Pichinde virus particle expressing an antigen derived from an infectious organism, a cancer, or an allergen as described herein or a composition thereof is administered to a subject having a recurrent infection, a cancer, or an allergy.

In another embodiment, a Pichinde virus particle or a tri-segmented Pichinde virus particle expressing an antigen derived from an infectious organism, a cancer, or an allergen as described herein or a composition thereof is administered to a subject with a genetic predisposition for an infection, a cancer, or an allergy. In another embodiment, a Pichinde virus particle or tri-segmented Pichinde virus particle expressing an antigen derived from an infectious organism, a cancer, or an allergen as described herein or a composition thereof is administered to a subject. In another embodiment, a Pichinde virus particle or a tri-segmented Pichinde virus particle expressing an antigen derived from an infectious organism, a cancer, or an allergen is administered to a subject with risk factors. Exemplary risk factors include, aging, tobacco, sun exposure, radiation exposure, chemical exposure, family history, alcohol, poor diet, lack of physical activity, or being overweight.

In another embodiment, administering a Pichinde virus particle or a tri-segmented Pichinde virus particle expressing an antigen derived from an infectious organism, a cancer, or an allergen reduces a symptomatic infection, cancer, or allergy. In another embodiment, administering a Pichinde virus particle or tri-segmented Pichinde virus particle expressing an antigen derived from an infectious organism, a cancer, or an allergen reduces an asymptomatic infection, cancer, or allergy.

In another embodiment, a Pichinde virus particle or a tri-segmented Pichinde virus particle expressing an antigen derived from an infectious organism described herein or a composition thereof is administered to subjects or animals infected with one or more strains of influenza virus, infectious bursal disease virus, rotavirus, infectious bronchitis virus, infectious laryngotracheitis virus, chicken anemia virus, Marek's disease virus, avian leukosis virus, avian adenovirus, or avian pneumovirus, SARS-causing virus, human respiratory syncytial virus, human immunodeficiency virus, hepatitis A virus, hepatitis B virus, hepatitis C virus, poliovirus, rabies virus, Hendra virus, Nipah virus, human parainfluenza 3 virus, measles virus, mumps virus, Ebola virus, Marburg virus, West Nile disease virus, Japanese encephalitis virus, Dengue virus, Hantavirus, Rift Valley fever virus, Lassa fever virus, herpes simplex virus and yellow fever virus.

In another embodiment, a Pichinde virus particle or a tri-segmented Pichinde virus particle expressing an antigen derived from a cancer described herein or a composition thereof is administered to subjects who suffer from one or more types of cancers. In other embodiments, any type of a cancer susceptible to treatment with the vaccines described herein might be targeted. In a more specific embodiment, a Pichinde virus particle or a tri-segmented Pichinde virus particle expressing an antigen derived from a cancer described herein or a composition thereof is administered to subjects suffering from, for example, melanoma, prostate carcinoma, breast carcinoma, lung carcinoma, neuroblastoma, hepatocellular carcinoma, cervical carcinoma, and stomach carcinoma, burkitt lymphoma; non-Hodgkin lymphoma; Hodgkin lymphoma; nasopharyngeal carcinoma (cancer of the upper part of the throat behind the nose), leukemia, mucosa-associated lymphoid tissue lymphoma.

In another embodiment, a Pichinde virus particle or a tri-segmented Pichinde virus particle expressing an antigen derived from an allergen described herein or a composition thereof is administered to subjects who suffer from one or more allergies. In a more specific embodiment, a Pichinde virus particle or a tri-segmented Pichinde virus particle expressing an antigen derived from an allergen described herein or a composition thereof is administered to subjects suffering from, for example, a seasonal allergy, a perennial allergy, rhinoconjunctivitis, asthma, eczema, a food allergy.

In another embodiment, administering a Pichinde virus particle or a tri-segmented Pichinde virus particle expressing an antigen derived from an infectious organism, a cancer, or an allergen as described herein or a composition thereof to subjects confer cell-mediated immunity (CMI) against an infection, a cancer, or an allergen. Without being bound by theory, in another embodiment, a Pichinde virus particle or a tri-segmented Pichinde virus particle expressing an antigen derived from an infectious organism, a cancer, an allergen as described herein or a composition thereof infects and expresses antigens of interest in antigen presenting cells (APC) of the host (e.g., macrophages, dendritic cells, or B cells) for direct presentation of antigens on Major Histocompatibility Complex (MHC) class I and II. In another embodiment, administering a Pichinde virus particle or a tri-segmented Pichinde virus particle expressing an antigen derived from an infectious organism, a cancer, an allergen as described herein or a composition thereof to subjects induces plurifunctional cytolytic as well as IFN-γ and TNF-α co-producing CMV-specific CD4+ and CD8+ T cell responses of high magnitude to treat or prevent an infection, a cancer, or an allergy.

In another embodiment, administering a Pichinde virus particle or a tri-segmented Pichinde virus particle expressing an antigen derived from an infectious organism, a cancer, or an allergen or a composition thereof reduces the risk that an individual will develop an infection, a cancer, an allergy by at least about 10%, at least about 20%, at least about 25%, at least about 30%, at least about 35%, at least about 40%, at least about 50%, at least about 60%, at least about 70%, at least about 80%, at least about 90%, or more, compared to the risk of developing an infection, a cancer, or an allergy in the absence of such treatment.

In another embodiment, administering a Pichinde virus particle or a tri-segmented Pichinde virus particle expressing an antigen derived from an infectious organism, a cancer, or an allergen or a composition thereof reduces the symptoms of an infection, a cancer, or an allergy by at least about 10%, at least about 20%, at least about 25%, at least about 30%, at least about 35%, at least about 40%, at least about 50%, at least about 60%, at least about 70%, at least about 80%, at least about 90%, or more, compared to the manifestation of the symptoms of an infection, a cancer, an allergy in the absence of such treatment.

In certain embodiments, the Pichinde virus particle or tri-segmented Pichinde virus particle expressing an antigen derived from an infectious organism, a cancer, or an allergen is preferably administered in multiple injections (e.g., at least 2, 3, 4, 5, 6, 7, 8, 9, 10, 12, 14, 16, 18, 20, 25, 30, 40, 45, or 50 injections) or by continuous infusion (e.g., using a pump) at multiple sites (e.g., at least 2, 3, 4, 5, 6, 7, 8, 9, 10, 12, or 14 sites). In certain embodiments, the Pichinde virus particle or tri-segmented Pichinde virus particle expressing an antigen derived from an infectious organism, a cancer, or an allergen is administered in two or more separate injections over a 6-month period, a 12-month period, a 24-month period, or a 48-month period. In certain embodiments, the Pichinde virus particle or tri-segmented Pichinde virus particle expressing an antigen derived from a infectious organism, a cancer, or an allergen is administered with a first dose at an elected date, a second dose at least 2 months after the first dose, and a third does 6 months after the first dose.

In one example, cutaneous injections are performed at multiple body sites to reduce extent of local skin reactions. On a given vaccination day, the patient receives the assigned total dose of cells administered from one syringe in 3 to 5 separate intradermal injections of the dose (e.g., at least 0.4 ml, 0.2 ml, or 0.1 ml) each in an extremity spaced at least about 5 cm (e.g., at least 4.5, 5, 6, 7, 8, 9, or cm) at needle entry from the nearest neighboring injection. On subsequent vaccination days, the injection sites are rotated to different limbs in a clockwise or counter-clockwise manner.

In another embodiment, administering an infectious, replication-deficient Pichinde virus expressing a CMV antigen or a composition thereof in subjects with a neonatal and therefore immune system induces a cell-mediated immune (CMI) response against an infection, a cancer, or an allergy, exceeding by at least about 10%, at least about 20%, at least about 25%, at least about 30%, at least about 35%, at least about 40%, at least about 50%, at least about 60%, at least about 70%, at least about 80%, at least about 90%, or more, the CMI response against an infection, a cancer, or a allergy in the absence of such a treatment.

In certain embodiments, administrating to a subject a Pichinde virus particle or a tri-segmented Pichinde virus particle expressing an antigen derived from an infectious organism, a cancer, or an allergen, as described herein induces a detectable antibody titer for a minimum of at least four weeks. In another embodiment, administering to a subject a Pichinde virus particle or a tri-segmented Pichinde virus particle expressing an antigen derived from an infectious organism, a cancer, or an allergen, as describe herein increases the antibody titer by at least 100%, at least 200%, at least 300%, at least 400%, at least 500%, or at least 1000%.

In certain embodiments, primary antigen exposure elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the primary neutralizing geometric mean antibody titer increases up to a peak value of at least 1:50, at least 1:100, at least 1:200, or at least 1:1000 within at least 4 weeks post-immunization. In another embodiment, immunization with a Pichinde virus particle or a tri-segmented Pichinde virus particle expressing an antigen derived from an infectious organism, a cancer, or an allergy, as described herein produces high titers of antibodies that last for at least 4 weeks, at least 8 weeks, at least 12 weeks, at least 6 months, at least 12 months, at least 2 years, at least 3 years, at least 4 years, or at least 5 years post-immunization following a single administration of the vaccine, or following two or more sequential immunizations.

In yet another embodiment, secondary antigen exposure increases the antibody titer by at least 100%, at least 200%, at least 300%, at least 400%, at least 500%, or at least 1000%. In another embodiment, secondary antigen exposure elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the secondary neutralizing geometric mean antibody titer increases up to a peak value of at least 1:50, at least 1:100, at least 1:200, or at least 1:1000 within at least 4 weeks post-immunization. In another embodiment, a second immunization with a Pichinde virus particle or a tri-segmented Pichinde virus particle expressing an antigen derived from an infectious organism, a cancer, or an allergy, as described herein produces high titers of antibodies that last for at least 4 weeks, at least 8 weeks, at least 12 weeks, at least 6 months, at least 12 months, at least 2 years, at least 3 years, at least 4 years, or at least 5 years post-immunization.

In yet another embodiment, a third boosting immunization increases the antibody titer by at least 100%, at least 200%, at least 300%, at least 400%, at least 500%, or at least 1000%. In another embodiment, the boosting immunization elicits a functional, (neutralizing) and minimum antibody titer of at least 50%, at least 100%, at least 200%, at least 300%, at least 400%, at least 500%, or at least 1000% of mean control sera from infection-immune human subjects. In more specific embodiments, the third boosting immunization elicits a functional, (neutralizing), and minimum antibody titer of at least 50%, at least 100%, at least 200%, at least 300%, at least 400%, at least 500%, or at least 1000% of mean control sera from infection-immune human subjects. In another embodiment, a third boosting immunization prolongs the antibody titer by at least 4 weeks, at least 8 weeks, at least 12 weeks, at least 6 months, at least 12 months, at least 2 years, at least 3 years, at least 4 years, or at least 5 years post-immunization

In certain embodiments, the Pichinde virus particle or a tri-segmented Pichinde virus particle expressing an antigen derived from an infectious organism, a cancer, or an allergy, elicits a T cell independent or T cell dependent response. In other embodiments, Pichinde virus particle or a tri-segmented Pichinde virus particle expressing an antigen derived from an infectious organism, a cancer, or an allergy, elicits a T cell response. In other embodiments, a Pichinde virus particle or a tri-segmented Pichinde virus particle expressing an antigen derived from an infectious organism, a cancer, or an allergy, as described herein elicits a T helper response. In another embodiment, Pichinde virus particle or a tri-segmented Pichinde virus particle expressing an antigen derived from an infectious organism, a cancer, or an allergy, as described herein elicits a Th1-orientated response or a Th2-orientated response.

In more specific embodiments, the Th1-orientated response is indicated by a predominance of IgG2 antibodies versus IgG1. In other embodiments the ratio of IgG2:IgG1 is greater than 1:1, greater than 2:1, greater than 3:1, or greater than 4:1. In another embodiment the infectious, Pichinde virus particle or a tri-segmented Pichinde virus particle expressing an antigen derived from an infectious organism, a cancer, or an allergy, as described herein is indicated by a predominance of IgG1, IgG2, IgG3, IgG4, IgM, IgA or IgE antibodies.

In some embodiments, the infectious, replication-deficient Pichinde virus expressing a CMV antigen or a fragment thereof elicits a CD8+ T cell response. In another embodiment, the Pichinde virus particle or a tri-segmented Pichinde virus particle expressing an antigen derived from an infectious organism, a cancer, or an allergy elicits both CD4+ and CD8+ T cell responses, in combination with antibodies or not.

In certain embodiments, the Pichinde virus particle or a tri-segmented Pichinde virus particle expressing an antigen derived from an infectious organism, a cancer, or an allergy, as described herein elicits high titers of neutralizing antibodies. In another embodiment, the Pichinde virus particle or a tri-segmented Pichinde virus particle expressing an antigen derived from an infectious organism, a cancer, or an allergy, as described herein elicits higher titers of neutralizing antibodies than expression of the protein complex components individually.

In another embodiment, the Pichinde virus particle or a tri-segmented Pichinde virus particle expressing one, two, three, four, five, or more antigen derived from an infectious organism, a cancer, or an allergy elicits higher titers of neutralizing antibodies than a Pichinde virus particle or a tri-segmented Pichinde virus particle expressing one expressing one antigen derived from an infectious organism, a cancer, or an allergen.

In certain embodiments, the methods further comprise co-administration of the Pichinde virus particle or tri-segmented Pichinde virus particle and at least one additional therapy. In certain embodiments, the co-administration is simultaneous. In another embodiment, the Pichinde virus particle or tri-segmented Pichinde virus particle is administered prior to administration of the additional therapy. In other embodiments, the Pichinde virus particle or tri-segmented Pichinde virus particle is administered after administration of the additional therapy. In certain embodiments, the administration of the Pichinde virus particle or tri-segmented Pichinde virus particle and the additional therapy is about 1 hour, about 2 hours, about 3 hours, about 4 hours, about 5 hours, about 6 hours, about 7 hours, about 8 hours, about 9 hours, about 10 hours, about 11 hours, or about 12 hours. In certain embodiments, the interval between administration of the Pichinde virus particle or tri-segmented Pichinde virus particle and said additional therapy is about 1 day, 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks. In certain embodiments, the interval between administration of the Pichinde virus particle or tri-segmented Pichinde virus particle and the additional therapy is about 1 month, about 2 months, about 3 months, about 4 months, about 5 months, or about 6 months.

In certain embodiments, administering a Pichinde virus particle expressing an antigen derived from an infectious organism, a cancer, or an allergen or a composition thereof reduces the number of antibodies detected in a patient blood sample, or serum sample. In certain embodiments, administering a Pichinde virus particle expressing an antigen derived from an infectious organism, a cancer, or an allergen composition thereof reduces the amount of the infectious organism, cancer, or allergy detected in urine, saliva, blood, tears, semen, exfoliated cell sample, or breast milk.

In another embodiment, the Pichinde virus particle or the tri-segmented Pichinde virus particle expressing an antigen derived from an infection organism, a cancer, or an allergen as described herein or a composition may further comprise a reporter protein. In a more specific embodiment, the, the Pichinde virus particle or a tri-segmented Pichinde virus particle expressing an antigen derived from an infection organism, a cancer, or an allergen and reporter protein as described herein or a composition is administered to subjects for treating and/or preventing an infection, a cancer, or an allergy. In yet another specific embodiment, the reporter protein can be used for monitoring gene expression, protein localization, and vaccine delivery, in vivo, in situ and in real time.

In another embodiment, the Pichinde virus particle or a tri-segmented Pichinde virus particle expressing an antigen derived from an infection organism, a cancer, or an allergen as described herein or a composition may further comprise a fluorescent protein. In a more specific embodiment, the Pichinde virus particle or a tri-segmented Pichinde virus particle expressing an antigen derived from an infection organism, a cancer, or an allergen and reporter protein as described herein or a composition is administered to subjects for treating and/or preventing an infection, a cancer, or an allergy. In yet another specific embodiment, the fluorescent protein can be the reporter protein can be used for monitoring gene expression, protein localization, and vaccine delivery, in vivo, in situ and in real time.

Changes in the CMI response function against an infection, a cancer, or an allergy induced by administering a Pichinde virus particle or a tri-segmented Pichinde virus particle expressing an antigen derived from an infectious organism, a cancer, an allergen or a composition thereof in subjects can be measured by any assay known to the skilled artisan including, but not limited to flow cytometry (see, e.g., Perfetto S. P. et al., 2004, Nat Rev Immun., 4(8):648-55), lymphocyte proliferation assays (see, e.g., Bonilla F. A. et al., 2008, Ann Allergy Asthma Immunol, 101:101-4; and Hicks M. J. et al., 1983, Am J Clin Pathol., 80:159-63), assays to measure lymphocyte activation including determining changes in surface marker expression following activation of measurement of cytokines of T lymphocytes (see, e.g., Caruso A. et al., Cytometry. 1997; 27:71-6), ELISPOT assays (see, e.g., Czerkinsky C. C. et al., 1983, J Immunol Methods, 65:109-121; and Hutchings P. R. et al., 1989, J Immunol Methods, 120:1-8), or Natural killer cell cytotoxicity assays (see, e.g., Bonilla F. A. et al., 2006, Ann Allergy Asthma Immunol., 94(5 Suppl 1):S1-63).

Successful treatment of a cancer patient can be assessed as prolongation of expected survival, induction of an anti-tumor immune response, or improvement of a particular characteristic of a cancer. Examples of characteristics of a cancer that might be improved include tumor size (e.g., T0, T is, or T1-4), state of metastasis (e.g., M0, M1), number of observable tumors, node involvement (e.g., N0, N1-4, Nx), grade (i.e., grades 1, 2, 3, or 4), stage (e.g., 0, I, II, III, or IV), presence or concentration of certain markers on the cells or in bodily fluids (e.g., AFP, B2M, beta-HCG, BTA, CA 15-3, CA 27.29, CA 125, CA 72.4, CA 19-9, calcitonin, CEA, chromgrainin A, EGFR, hormone receptors, HER2, HCG, immunoglobulins, NSE, NMP22, PSA, PAP, PSMA, S-100, TA-90, and thyroglobulin), and/or associated pathologies (e.g., ascites or edema) or symptoms (e.g., cachexia, fever, anorexia, or pain). The improvement, if measureable by percent, can be at least 5, 10, 15, 20, 25, 30, 40, 50, 60, 70, 80, or 90% (e.g., survival, or volume or linear dimensions of a tumor).

In another embodiment, described herein, is a method of use with a Pichinde virus particle expressing an antigen derived from an infectious organism, a cancer, or an allergen as described herein in which the at least one of the ORF encoding the GP, NP, Z protein, and L protein is substituted with a nucleotide sequence encoding an infectious a nucleotide sequence encoding an antigen derived from an infectious organism, a cancer, an allergen, or an antigenic fragment thereof.

### 4.7 Compositions, Administration, and Dosage

The present application furthermore relates to vaccines, immunogenic compositions (e.g., vaccine formulations), and pharmaceutical compositions comprising a Pichinde virus particle or a tri-segmented Pichinde virus particle as described herein. Such vaccines, immunogenic compositions and pharmaceutical compositions can be formulated according to standard procedures in the art.

It will be readily apparent to one of ordinary skill in the relevant arts that suitable modifications and adaptations to the methods and applications described herein can be obvious and can be made without departing from the scope of the scope or any embodiment thereof.

In another embodiment, provided herein are compositions comprising a Pichinde virus particle or a tri-segmented Pichinde virus particle described herein. Such compositions can be used in methods of treatment and prevention of disease. In a specific embodiment, the compositions described herein are used in the treatment of subjects infected with, or susceptible to, an infection. In other embodiments, the compositions described herein are used in the treatment of subjects susceptible to or exhibiting symptoms characteristic of cancer or tumorigenesis or are diagnosed with cancer. In another specific embodiment, the immunogenic compositions provided herein can be used to induce an immune response in a host to whom the composition is administered. The immunogenic compositions described herein can be used as vaccines and can accordingly be formulated as pharmaceutical compositions. In a specific embodiment, the immunogenic compositions described herein are used in the prevention of infection or cancer of subjects (e.g., human subjects). In other embodiments, the vaccine, immunogenic composition or pharmaceutical composition are suitable for veterinary and/or human administration.

In certain embodiments, provided herein are immunogenic compositions comprising a Pichinde virus vector as described herein. In certain embodiments, such an immunogenic composition further comprises a pharmaceutically acceptable excipient. In certain embodiments, such an immunogenic composition further comprises an adjuvant. The adjuvant for administration in combination with a composition described herein may be administered before, concomitantly with, or after administration of said composition. In some embodiments, the term “adjuvant” refers to a compound that when administered in conjunction with or as part of a composition described herein augments, enhances and/or boosts the immune response to a Pichinde virus particle or tri-segmented Pichinde virus particle and, most importantly, the gene products it vectorises, but when the compound is administered alone does not generate an immune response to the Pichinde virus particle or tri-segmented Pichinde virus particle and the gene products vectorised by the latter. In some embodiments, the adjuvant generates an immune response to the Pichinde virus particle or tri-segmented Pichinde virus particle and the gene products vectorised by the latter and does not produce an allergy or other adverse reaction. Adjuvants can enhance an immune response by several mechanisms including, e.g., lymphocyte recruitment, stimulation of B and/or T cells, and stimulation of macrophages or dendritic cells. When a vaccine or immunogenic composition of the invention comprises adjuvants or is administered together with one or more adjuvants, the adjuvants that can be used include, but are not limited to, mineral salt adjuvants or mineral salt gel adjuvants, particulate adjuvants, microparticulate adjuvants, mucosal adjuvants, and immunostimulatory adjuvants. Examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), ASO4 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., 1995, in Vaccine Design: The Subunit and Adjuvant Approach (eds. Powell & Newman, Plenum Press, NY); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al., 1997, N. Engl. J. Med. 336, 86-91).

The compositions comprise the Pichinde viruses particle or tri-segmented Pichinde virus particle described herein alone or together with a pharmaceutically acceptable carrier. Suspensions or dispersions of the Pichinde virus particle or tri-segmented Pichinde virus particle, especially isotonic aqueous suspensions or dispersions, can be used. The pharmaceutical compositions may be sterilized and/or may comprise excipients, e.g., preservatives, stabilizers, wetting agents and/or emulsifiers, solubilizers, salts for regulating osmotic pressure and/or buffers and are prepared in a manner known per se, for example by means of conventional dispersing and suspending processes. In certain embodiments, such dispersions or suspensions may comprise viscosity-regulating agents. The suspensions or dispersions are kept at temperatures around 2° C. to 8° C., or preferentially for longer storage may be frozen and then thawed shortly before use, or alternatively may be lyophilized for storage. For injection, the vaccine or immunogenic preparations may be formulated in aqueous solutions, preferably in physiologically compatible buffers such as Hanks's solution, Ringer's solution, or physiological saline buffer. The solution may contain formulatory agents such as suspending, stabilizing and/or dispersing agents.

In certain embodiments, the compositions described herein additionally comprise a preservative, e.g., the mercury derivative thimerosal. In a specific embodiment, the pharmaceutical compositions described herein comprise 0.001% to 0.01% thimerosal. In other embodiments, the pharmaceutical compositions described herein do not comprise a preservative.

The pharmaceutical compositions comprise from about 10³ to about 10¹¹ focus forming units of the Pichinde virus particle or tri-segmented Pichinde virus particle.

In one embodiment, administration of the pharmaceutical composition is parenteral administration. Parenteral administration can be intravenous or subcutaneous administration. Accordingly, unit dose forms for parenteral administration are, for example, ampoules or vials, e.g., vials containing from about 10³ to 10¹⁰ focus forming units or 10⁵ to 10¹⁵ physical particles of the Pichinde virus particle or tri-segmented Pichinde virus particle. In certain embodiments, the term “10eX” means 10 to the power of X.

In another embodiment, a vaccine or immunogenic composition provided herein is administered to a subject by, including but not limited to, oral, intradermal, intramuscular, intraperitoneal, intravenous, topical, subcutaneous, percutaneous, intranasal and inhalation routes, and via scarification (scratching through the top layers of skin, e.g., using a bifurcated needle). Specifically, subcutaneous or intravenous routes can be used.

For administration intranasally or by inhalation, the preparation for use according to the present invention can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflators may be formulated containing a powder mix of the compound and as suitable powder base such as lactose or starch.

The dosage of the active ingredient depends upon the type of vaccination and upon the subject, and their age, weight, individual condition, the individual pharmacokinetic data, and the mode of administration. In certain embodiments, an in vitro assay is employed to help identify optimal dosage ranges. Effective doses may be extrapolated from dose response curves derived from in vitro or animal model test systems.

In certain embodiments, the vaccine, immunogenic composition, or pharmaceutical composition comprising a Pichinde virus particle or the tri-segmented Pichinde virus particle can be used as a live vaccination. Exemplary doses for a live Pichinde virus particle may vary from 10-100, or more, PFU of live virus per dose. In some embodiments, suitable dosages of a Pichinde virus particle or the tri-segmented Pichinde virus particle are 10², 5×10², 10³, 5×10³, 10⁴, 5×10⁴, 10⁵, 5×10⁵, 10⁶, 5×10⁶, 10⁷, 5×10⁷, 10⁸, 5×10⁸, 1×10⁹, 5×10⁹, 5×10¹⁰, 1×10¹¹, 5×10¹¹ or 10¹² pfu, and can be administered to a subject once, twice, three or more times with intervals as often as needed. In another embodiment, a live Pichinde virus is formulated such that a 0.2-mL dose contains 10^{6.5}-10^{7.5} fluorescent focal units of live Pichinde virus particle. In another embodiment, an inactivated vaccine is formulated such that it contains about 15 μg to about 100 μg, about 15 μg to about 75 μg, about 15 μg to about 50 μg, or about 15 μg to about 30 μg of a Pichinde virus

In certain embodiments, for administration to children, two doses of a Pichinde virus particle or a tri-segmented Pichinde virus particle described herein or a composition thereof, given at least one month apart, are administered to a child. In specific embodiments for administration to adults, a single dose of the Pichinde virus particle or tri-segmented Pichinde virus particle described herein or a composition thereof is given. In another embodiment, two doses of a Pichinde virus particle or a tri-segmented Pichinde virus particle described herein or a composition thereof, given at least one month apart, are administered to an adult. In another embodiment, a young child (six months to nine years old) may be administered a Pichinde virus particle or a tri-segmented Pichinde virus particle described herein or a composition thereof for the first time in two doses given one month apart. In a particular embodiment, a child who received only one dose in their first year of vaccination should receive two doses in the following year. In some embodiments, two doses administered 4 weeks apart are preferred for children 2-8 years of age who are administered an immunogenic composition described herein, for the first time. In certain embodiments, for children 6-35 months of age, a half dose (0.25 ml) may be preferred, in contrast to 0.5 ml which may be preferred for subjects over three years of age.

In certain embodiments, the compositions can be administered to the patient in a single dosage comprising a therapeutically effective amount of the Pichinde virus particle or the tri-segmented Pichinde virus particle. In some embodiments, the Pichinde virus particle or tri-segmented Pichinde virus particle can be administered to the patient in a single dose comprising a therapeutically effective amount of a Pichinde virus particle or tri-segmented Pichinde virus particle and, one or more pharmaceutical compositions, each in a therapeutically effective amount.

In certain embodiments, the composition is administered to the patient as a single dose followed by a second dose three to six weeks later. In accordance with these embodiments, the booster inoculations may be administered to the subjects at six to twelve month intervals following the second inoculation. In certain embodiments, the booster inoculations may utilize a different Pichinde virus or composition thereof. In some embodiments, the administration of the same composition as described herein may be repeated and separated by at least 1 day, 2 days, 3 days, 4 days, 5 days, 10 days, 15 days, 30 days, 45 days, 2 months, 75 days, 3 months, or at least 6 months.

Also provided herein, are processes and to the use the Pichinde virus particle or the tri-segmented Pichinde virus particle for the manufacture of vaccines in the form of pharmaceutical preparations, which comprise the Pichinde virus particle or tri-segmented Pichinde virus particle as an active ingredient. The pharmaceutical compositions of the present application are prepared in a manner known per se, for example by means of conventional mixing and/or dispersing processes.

### 4.8 Assays

### 4.8.1 Pichinde Virus Detection Assays

The skilled artesian could detect a Pichinde virus genomic segment or tri-segmented Pichinde virus particle, as described herein using techniques known in the art. For example, RT-PCR can be used with primers that are specific to a Pichinde virus to detect and quantify a Pichinde virus genomic segment that has been engineered to carry an ORF in a position other than the wild-type position of the ORF or a tri-segmented Pichinde virus particle. Western blot, ELISA, radioimmunoassay, immuneprecipitation, immunecytochemistry, or immunocytochemistry in conjunction with FACS can be used to quantify the gene products of the Pichinde virus genomic segment or tri-segmented Pichinde virus particle.

### 4.8.2 Assay to Measure Infectivity

Any assay known to the skilled artisan can be used for measuring the infectivity of a Pichinde virus vector preparation. For example, determination of the virus/vector titer can be done by a “focus forming unit assay” (FFU assay). In brief, complementing cells, e.g., MC57 cells are plated and inoculated with different dilutions of a virus/vector sample. After an incubation period, to allow cells to form a monolayer and virus to attach to cells, the monolayer is covered with Methylcellulose. When the plates are further incubated, the original infected cells release viral progeny. Due to the Methylcellulose overlay the spread of the new viruses is restricted to neighboring cells. Consequently, each infectious particle produces a circular zone of infected cells called a Focus. Such Foci can be made visible and by that countable using antibodies against Pichinde virus—NP or another protein expressed by the Pichinde virus particle or the tri-segmented Pichinde virus particle and a HRP-based color reaction. The titer of a virus/vector can be calculated in focus-forming units per milliliter (FFU/mL).

### 4.8.3 Growth of a Pichinde Virus Particle

Growth of a Pichinde virus particle described herein can be assessed by any method known in the art or described herein (e.g., cell culture). Viral growth may be determined by inoculating serial dilutions of a Pichinde virus particle described herein into cell cultures (e.g., BHK-21 cells). After incubation of the virus for a specified time, the virus is isolated using standard methods.

### 4.8.4 Serum ELISA

Determination of the humoral immune response upon vaccination of animals (e.g., mice, guinea pigs) can be done by antigen-specific serum ELISA's (enzyme-linked immunosorbent assays). In brief, plates are coated with antigen (e.g., recombinant protein), blocked to avoid unspecific binding of antibodies and incubated with serial dilutions of sera. After incubation, bound serum-antibodies can be detected, e.g., using an enzyme-coupled anti-species (e.g., mouse, guinea pig)-specific antibody (detecting total IgG or IgG subclasses) and subsequent color reaction. Antibody titers can be determined as, e.g., endpoint geometric mean titer.

### 4.8.5 Assay to Measure the Neutralizing Activity of Induced Antibodies

Determination of the neutralizing antibodies in sera is performed with the following cell assay using ARPE-19 cells from ATCC and a GFP-tagged virus. In addition supplemental guinea pig serum as a source of exogenous complement is used. The assay is started with seeding of 6.5×10³ cells/well (50 μl/well) in a 384 well plate one or two days before using for neutralization. The neutralization is done in 96-well sterile tissue culture plates without cells for 1 h at 37° C. After the neutralization incubation step the mixture is added to the cells and incubated for additional 4 days for GFP-detection with a plate reader. A positive neutralizing human sera is used as assay positive control on each plate to check the reliability of all results. Titers (EC50) are determined using a 4 parameter logistic curve fitting. As additional testing the wells are checked with a fluorescence microscope.

### 4.8.6 Plaque Reduction Assay

In brief, plaque reduction (neutralization) assays for Pichinde virus can be performed by use of a replication-competent or -deficient Pichinde virus that is tagged with green fluorescent protein, 5% rabbit serum may be used as a source of exogenous complement, and plaques can be enumerated by fluorescence microscopy. Neutralization titers may be defined as the highest dilution of serum that results in a 50%, 75%, 90% or 95% reduction in plaques, compared with that in control (pre-immune) serum samples.

qPCR: Pichinde virus RNA genomes are isolated using QlAamp Viral RNA mini Kit (QIAGEN), according to the protocol provided by the manufacturer. Pichinde virus RNA genome equivalents are detected by quantitative PCR carried out on an StepOnePlus Real Time PCR System (Applied Biosystems) with SuperScript® III Platinum® One-Step qRT-PCR Kit (Invitrogen) and primers and probes (FAM reporter and NFQ-MGB Quencher) specific for part of the Pichinde NP coding region or another genomic stretch of the Pichinde virus particle or the tri-segmented Pichinde virus particle. The temperature profile of the reaction may be: 30 min at 60° C., 2 min at 95° C., followed by 45 cycles of 15 s at 95° C., 30 s at 56° C. RNA can be quantified by comparison of the sample results to a standard curve prepared from a log 10 dilution series of a spectrophotometrically quantified, in vitro-transcribed RNA fragment, corresponding to a fragment of the NP coding sequence or another genomic stretch of the Pichinde virus particle or the tri-segmented Pichinde virus particle containing the primer and probe binding sites.

### 4.8.7 Western Blotting

Infected cells grown in tissue culture flasks or in suspension are lysed at indicated timepoints post infection using RIPA buffer (Thermo Scientific) or used directly without cell-lysis. Samples are heated to 99° C. for 10 minutes with reducing agent and NuPage LDS Sample buffer (NOVEX) and chilled to room temperature before loading on 4-12% SDS-gels for electrophoresis. Proteins are blotted onto membranes using Invitrogens iBlot Gel transfer Device and visualized by Ponceau staining. Finally, the preparations are probed with a primary antibodies directed against proteins of interest and alkaline phosphatase conjugated secondary antibodies followed by staining with 1-Step NBT/BCIP solution (INVITROGEN).

### 4.8.8 MHC-Peptide Multimer Staining Assay for Detection of Antigen-Specific CD8+ T-Cell Proliferation

Any assay known to the skilled artisan can be used to test antigen-specific CD8+ T-cell responses. For example, the MHC-peptide tetramer staining assay can be used (see, e.g., Altman J. D. et al., Science. 1996; 274:94-96; and Murali-Krishna K. et al., Immunity. 1998; 8:177-187). Briefly, the assay comprises the following steps, a tetramer assay is used to detect the presence of antigen specific T-cells. In order for a T-cell to detect the peptide to which it is specific, it must both recognize the peptide and the tetramer of MHC molecules custom made for a defined antigen specificity and MHC haplotype of T-cells (typically fluorescently labeled). The tetramer is then detected by flow cytometry via the fluorescent label.

### 4.8.9 ELISPOT Assay for Detection of Antigen-Specific CD4+ T-Cell Proliferation.

Any assay known to the skilled artisan can be used to test antigen-specific CD4+ T-cell responses. For example, the ELISPOT assay can be used (see, e.g., Czerkinsky C. C. et al., J Immunol Methods. 1983; 65:109-121; and Hutchings P. R. et al., J Immunol Methods. 1989; 120:1-8). Briefly, the assay comprises the following steps: An immunospot plate is coated with an anti-cytokine antibody. Cells are incubated in the immunospot plate. Cells secrete cytokines and are then washed off. Plates are then coated with a second biotyinlated-anticytokine antibody and visualized with an avidin-HRP system.

### 4.8.10 Intracellular Cytokine Assay for Detection of Functionality of CD8+ and CD4+ T-Cell Responses.

Any assay known to the skilled artisan can be used to test the functionality of CD8+ and CD4+ T cell responses. For example, the intracellular cytokine assay combined with flow cytometry can be used (see, e.g., Suni M. A. et al., J Immunol Methods. 1998; 212:89-98; Nomura L. E. et al., Cytometry. 2000; 40:60-68; and Ghanekar S. A. et al., Clinical and Diagnostic Laboratory Immunology. 2001; 8:628-63). Briefly, the assay comprises the following steps: activation of cells via specific peptides or protein, an inhibition of protein transport (e.g., brefeldin A) is added to retain the cytokines within the cell. After a defined period of incubation, typically 5 hours, a washing steps follows, and antibodies to other cellular markers can be added to the cells. Cells are then fixed and permeabilized. The flurochrome-conjugated anti-cytokine antibodies are added and the cells can be analyzed by flow cytometry.

### 4.8.11 Assay for Confirming Replication-Deficiency of Viral Vectors

Any assay known to the skilled artisan that determines concentration of infectious and replication-competent virus particles can also be used as a to measure replication-deficient viral particles in a sample. For example, FFU assays with non-complementing cells can be used for this purpose.

Furthermore, plaque-based assays are the standard method used to determine virus concentration in terms of plaque forming units (PFU) in a virus sample. Specifically, a confluent monolayer of non-complementing host cells is infected with the virus at varying dilutions and covered with a semi-solid medium, such as agar to prevent the virus infection from spreading indiscriminately. A viral plaque is formed when a virus successfully infects and replicates itself in a cell within the fixed cell monolayer, and spreads to surrounding cells (see, e.g., Kaufmann, S. H.; Kabelitz, D. (2002). Methods in Microbiology Vol. 32:Immunology of Infection. Academic Press. ISBN 0-12-521532-0). Plaque formation can take 2-14 days, depending on the virus being analyzed. Plaques are generally counted manually and the results, in combination with the dilution factor used to prepare the plate, are used to calculate the number of plaque forming units per sample unit volume (PFU/mL). The PFU/mL result represents the number of infective replication-competent particles within the sample. When C-cells are used, the same assay can be used to titrate replication-deficient Pichinde virus particles or tri-segmented Pichinde virus particles.

### 4.8.12 Assay for Expression of Viral Antigen

Any assay known to the skilled artisan can be used for measuring expression of viral antigens. For example, FFU assays can be performed. For detection, mono- or polyclonal antibody preparation(s) against the respective viral antigens are used (transgene-specific FFU).

### 4.8.13 Animal Models

To investigate recombination and infectivity of a Pichinde virus particle described herein in vivo animal models can be used. In certain embodiments, the animal models that can be used to investigate recombination and infectivity of a tri-segmented Pichinde virus particle include mouse, guinea pig, rabbit, and monkeys. In a preferred embodiment, the animal models that can be used to investigate recombination and infectivity of a Pichinde virus include mouse. In a more specific embodiment, the mice can be used to investigate recombination and infectivity of a Pichinde virus particle are triple-deficient for type I interferon receptor, type II interferon receptor and recombination activating gene 1 (RAG1).

In certain embodiments, the animal models can be used to determine Pichinde virus infectivity and transgene stability. In some embodiments, viral RNA can be isolated from the serum of the animal model. Techniques are readily known by those skilled in the art. The viral RNA can be reverse transcribed and the cDNA carrying the Pichinde virus ORFs can be PCR-amplified with gene-specific primers. Flow cytometry can also be used to investigate Pichinde virus infectivity and transgene stability.

### 5. EXAMPLES

These examples demonstrate that Pichinde virus-based vector technology can be used to successfully develop (1) an Pichinde virus genomic segment with a viral ORF in a position other than the wild-type position of the ORF, and (2) a tri-segmented Pichinde virus particle that does not result in a replication competent bi-segmented viral particle.

### 5.1 Materials and Methods

### 5.1.1 Cells

BHK-21 cells were cultured in high-glucose Dulbecco's Eagle medium (DMEM; Sigma) supplemented with 10% heat-inactivated fetal calf serum (FCS; Biochrom), 10 mM HEPES (Gibco), 1 mM sodium pyruvate (Gibco) and 1×tryptose phosphate broth. Cells were cultured at 37° C. in a humidified 5% CO2 incubator. 293-T cells were cultured in Dulbecco's Eagle medium (DMEM, containing Glutamax; Sigma) supplemented with 10% heat-inactivated fetal calf serum (FCS).

### 5.1.2 Transgenes

(1) Green fluorescent protein (GFP) was synthesized as GFP-Bsm (SEQ ID NO.: 9) with flanking BsmBI sites for seamless cloning. (2) A fusion protein consisting of i) the vesicular stomatitis virus glycoprotein (VSVG) signal peptide, ii) the P1A antigen of the P815 mouse mastocytoma tumor cell line, iii) a GSG linker, iv) an enterovirus 2A peptide, and v) mouse GM-CSF. This fusion protein will be referred to as sP1AGM. We synthesized it with flanking BsmBI sites as sP1AGM-Bsm (SEQ ID NO.: 10) for seamless cloning. (3) The Pichinde virus GP with flanking BsmBI sites for seamless cloning to reconstitute a wild type Pichinde virus S segment expression plasmid (S segment devoid of BbsI sites) (SEQ ID NO.: 8).

### 5.1.3 Plasmids

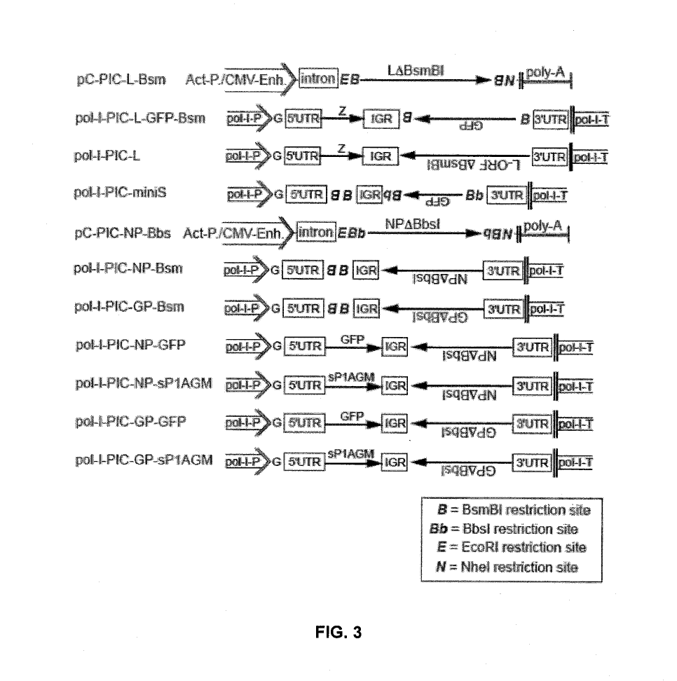

We synthesized a modified cDNA of the L ORF of Pichinde virus strain Munchique CoAn4763 isolate P18 (Genbank accession number EF529747.1), wherein a non-coding mutation was introduced to delete the BsmBI restriction site. This synthetic ORF with suitably flanking BsmBI as well as EcoRI and NheI restriction sites (LABsmBI; SEQ ID NO: 3) was introduced into the polymerase-II (pol-II) expression vector pCAGGS, yielding pC-PIC-L-Bsm (FIG. 3) for expression of the Pichinde L protein in eukaryotic cells.

We synthesized a modified L segment (PIC-L-GFP-Bsm; SEQ ID NO: 4) of Pichinde virus strain Munchique CoAn4763 isolate P18 (Genbank accession number EF529747.1), wherein the L ORF was deleted and substituted by a GFP ORF with flanking BsmBI sites on each side. This synthetic cDNA was introduced into a mouse polymerase I (pol-I) expression cassette (Pinschewer et al. J Virol. 2003 March; 77(6):3882-7), yielding pol-I-PIC-L-GFP-Bsm (FIG. 3).

We digested PIC-L-Bsm with BsmBI to insert the BsmBI-mutated L ORF into the equally digested pol-I-PIC-L-GFP-Bsm backbone, thereby replacing the GFP ORF with the L ORF to seamlessly reconstitute the Pichinde virus L segment cDNA, with all restriction sites for cloning purposes removed. The resulting pol-I-PIC-L plasmid (FIG. 3) was designed for intracellular expression of a full-length Pichinde Virus L segment (PIC-L-seg; SEQ ID NO.: 2) in eukaryotic cells.

We synthesized a modified S segment cDNA of Pichinde virus strain Munchique CoAn4763 isolate P18 (Genbank accession number: EF529746.1), referred to as PIC-miniS-GFP (SEQ ID NO: 5) wherein the GP ORF was replaced by two BsmBI restriction sites and the NP ORF was replaced by GFP with two flanking BbsI restriction sites. This synthetic cDNA was introduced into a mouse polymerase I (pol-I) expression cassette (Pinschewer et al. J Virol. 2003 March; 77(6):3882-7), yielding pol-I-PIC-miniS-GFP (FIG. 3).

We synthesized a modified cDNA of the NP ORF of Pichinde virus strain Munchique CoAn4763 isolate P18 (Genbank accession number EF529747.1), wherein non-coding mutation were introduced to delete both BbsI restriction sites. This synthetic ORF with suitably flanking BbsI as well as EcoRI and NheI restriction sites (NPABbsI; SEQ ID NO: 6) was introduced into the polymerase-II (pol-II) expression vector pCAGGS, yielding pC-PIC-NP-Bbs (FIG. 3) for expression of the Pichinde NP protein in eukaryotic cells.

We digested NPABbsI with BbsI to insert the BbsI-mutated NP ORF into the equally digested pol-I-PIC-miniS-GFP backbone, thereby replacing the GFP ORF with the NP ORF to seamlessly reconstitute the 3′UTR-NP-IGR portion of the Pichinde virus S segment cDNA, with all restriction sites for cloning purposes removed. The resulting pol-I-PIC-NP-Bsm plasmid (FIG. 3), expressing PIC-NP-Bsm (SEQ ID NO: 7) under control of pol-I, was designed for accepting transgenes of interest, to be inserted between the 5′UTR and the IGR, by seamlessly replacing the BsmBI sites, for expression of the resulting recombinant Pichinde virus S segment in eukaryotic cells.

We synthesized a modified cDNA of the GP ORF of Pichinde virus strain Munchique CoAn4763 isolate P18 (Genbank accession number EF529747.1), wherein non-coding mutation were introduced to delete both BbsI restriction sites. Analogously to NPABbsI, this synthetic ORF was introduced into the pol-I-PIC-miniS-GFP backbone, thereby replacing the GFP ORF with the GP ORF to seamlessly reconstitute a 3′UTR-GP-IGR portion of the Pichinde virus S segment cDNA, with all restriction sites for cloning purposes removed. The resulting pol-I-PIC-GP-Bsm plasmid (FIG. 3), expressing PIC-GP-Bsm (SEQ ID NO: 8), was designed for accepting transgenes of interest, to be inserted between the 5′UTR and the IGR, by seamlessly replacing the BsmBI sites, for expression of a recombinant Pichinde virus S segment in eukaryotic cells.

We then inserted into pol-I-PIC-NP-Bsm the following genes and transgenes: 1. GFP, 2. sP1AGM, and 3. Pichinde GP all with flanking BsmBI sites. The resulting plasmids were denominated pol-I-PIC-NP-GFP (expressing PIC-NP-GFP, also known as S-NP/GFP; SEQ ID NO: 11) and pol-I-PIC-NP-sP1AGM (expressing PIC-NP-sP1AGM; SEQ ID NO: 12) and pol-I-PIC-S (expressing PIC-S, SEQ ID NO: 1). Analogously we inserted either GFP or sP1AGM into pol-I-PIC-GP-Bsm, yielding pol-I-PIC-GP-GFP (expressing PIC-GP-GFP, also known as S-GP/GFPart; SEQ ID NO: 13) and pol-I-PIC-GP-sP1AGM (expressing PIC-GP-sP1AGM; SEQ ID NO: 14).

### 5.1.4 DNA Transfection of Cells and Rescue of Recombinant Viruses

BHK-21 cells stably transfected to express the glycoprotein of lymphocytic choriomeningitis virus (BHK-GP cells, Flatz et al. Nat Med. 2010 March; 16(3):339-45) were seeded into 6-well plates at a density of 5×10⁵ cells/well and transfected 24 hours later with different amounts of DNA using either lipofectamine (approx. 3 μl/μg DNA; Invitrogen) according to the manufacturer's instructions. For rescue of recombinant bi-segmented viruses entirely from plasmid DNA, the two minimal viral trans-acting factors NP and L were delivered from pol-II driven plasmids (0.8 μg pC-PIC-NP-Bbs, 1.4 μg pC-PIC-L-Bsm) and were co-transfected with 1 μg of pol-I-PIC-L and 0.8 μg of pol-I-PIC-S. In case of rescue of tri-segmented r3PIC consisting of one L and two S segments, 0.8 μg of both pol-I driven S segments were included in the transfection mix. 72 hours after transfection the cells and supernatant were transferred to a 75 cm2 tissue culture flask, and supernatant was harvested another 48-96 hours later. Viral infectivity was determined in a focus forming assay and the virus was passaged for 48 on normal BHK-21 cells for further amplification (multiplicity of infection=0.01 for 48 hours). Viral titers in the so obtained virus stocks were again determined by focus forming assay.

### 5.1.5 Viruses and Growth Kinetics of Viruses

Stocks of wild-type and recombinant viruses were produced by infecting either BHK-21 or 293-T cells at a multiplicity of infection (moi) of 0.01 and supernatant was harvested 48 hours after infection. Growth curves of viruses were done in vitro in T75 cell culture flask format. BHK021 cells were seeded at a density of 5×10⁶ cells/flask and infected 24 hours later by incubating the cells together with 5 ml of the virus inoculum at a moi of 0.01 for 90 minutes on a rocker plate at 37° C. and 5% CO2. Fresh medium was added and cells incubated at 37° C./5% CO2. Supernatant was taken at given time points (normally 24, 48, 72 hours) and viral titers analyzed by focus forming assay.

### 5.1.6 Focus Forming Assay

Next, titers of Pichinde virus are determined by focus forming assay. 293-T cells or 3T3 cells were used for focus forming assay if not stated otherwise. Cells were seeded at a density of 3×10⁴ cells per well in a 96-well plate and mixed with 100 μl of 3.17-fold serial dilutions of virus prepared in MEM/2% FCS. After 2-4 hours of incubation at 37° C., 80 μl of a viscous medium (2% Methylcellulose in 2× supplemented DMEM) were added per well to ensure spreading of viral particles only to neighboring cells. After 48 hours at 37° C. the supernatant was flicked off and cells were fixed by adding 100 μl of methanol for 20 minutes at room temperature (all following steps are performed at room temperature). Cells were permeabilised with 100 μl per well of BSS/1% Triton X-100 (Merck Millipore) for 20 minutes and subsequently blocked for 60 minutes with PBS/5% FCS. For anti-NP staining a rat anti-Pichinde-NP monoclonal antibody was used as a primary staining antibody, diluted in PBS/2.5% FCS for 60 minutes. Plates were washed three times with tap water and the secondary HRP-goat-anti-rat-IgG was added at a dilution of 1:100 in PBS/2.5% FCS and incubated for 1 hour. The plate was again washed three times with tap water. The color reaction (0.5 g/l DAB (Sigma D-5637), 0.5 g/l Ammonium Nickel sulfate in PBS/0.015% H2O2) was added and the reaction was stopped after 10 minutes with tap water. Stained foci were counted and the final titer calculated according to the dilution.

### 5.1.7 Mice

BALB/c mice were purchased from Charles River Laboratories and housed under specific pathogen-free (SPF) conditions for experiments. All animal experiments were performed at the University of Basel in accordance with the Swiss law for animal protection and the permission of the respective responsible cantonal authorities. Infection of the mice was done intravenously at a dose of 1×10⁵ FFU per mouse.

### 5.1.8 Flow Cytometry

Blood was stained with MHC class I tetramers loaded with the immunodominant P1A-derived H-2L^{d}-restricted epitope LPYLGWLVF (Aa35-43), in combination with anti-CD8a and anti-B220 antibodies, and epitope-specific CD8+ T cell frequencies were determined on a BD LSRFortessa flow cytometer and the data processed using FlowJo software (Tree Star, Ashland, Oreg.).

### 5.1.9 Statistical Analysis

Statistical significance was determined by two-tailed unpaired t test using Graphpad Prism software (version 6.0d).

### 5.2 Results

5.2.1 Design of Trisegmented Pichinde Virus-Based Vectors with an Artificial Genome Organization

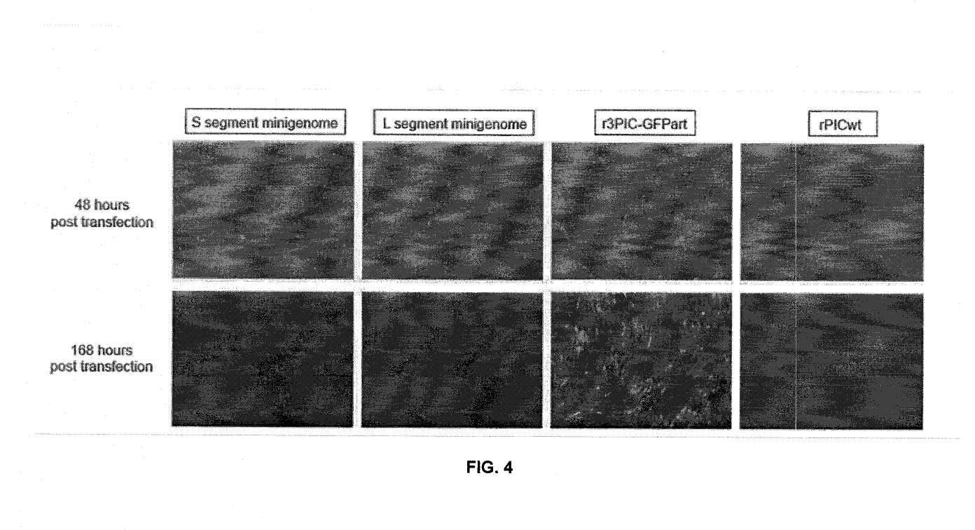

The genome of wild-type Pichinde virus consists of two single-stranded RNA segments of negative polarity (one L, one S segment) (FIG. 1A). We designed a polymerase-I/II-driven cDNA rescue system for replication-competent, tri-segmented Pichinde virus vectors with an artificial genome organization (r3PIC-art, FIGS. 1B, 1C and 1D), based on a cassette system allowing the seamless insertion of transgenes of choice between the 5′ untranslated region (5′UTR) and the intergenic region (IGR) of duplicated S segments. The molecular cloning strategy for seamless insertion (i.e. without residual nucleotide stretches derived from molecular cloning, and thus without additional restriction enzyme recognition sites) of transgenes into arenavirus S segments using BsmBI sites, which are completely removed upon transgene insertion and thus are absent from the resulting recombinant virus, has been described in detail by Pinschewer et al. Proc Natl Acad Sci USA. 2003 Jun. 24; 100(13):7895-900 in Supporting FIG. 4. The BbsI enzyme was used analogously for seamless cloning, as outlined by Flick et al. J Virol. 2001 February; 75(4):1643-55. These Pichinde virus-based r3PIC-art genomes consisted of the wild type Pichinde virus L segment together with artificially duplicated S segments, designed to carry either the nucleoprotein (NP) or the glycoprotein (GP) under control of the 3′UTR, i.e. between the 3′UTR and the IGR. This left in each S segment one position for insertion of a transgene, i.e. one transgene each could be inserted between the 5′UTR and IGR of each of the two S segments, respectively.

5.2.2 Infectious GFP-Expressing Virus Rescued from Trisegmented Recombinant Virus Vectors with an Artificial Genome Organization

To generate trisegmented recombinant Pichinde virus, we synthesized multiple plasmids as described in section 5.1.3. We transfected BHK-21 cells with plasmid combinations as follows:

(A) S segment minigenome: pC-PIC-L-Bsm, pC-PIC-NP-Bbs, pol-I-PIC-miniS-GFP;
(B) L segment minigenome: pC-PIC-L-Bsm, pC-PIC-NP-Bbs, pol-I-PIC-L-GFP-Bsm;
(C) r3PIC-GFP^{art}: pC-PIC-L-Bsm, pC-PIC-NP-Bbs, pol-I-PIC-L, pol-I-PIC-NP-GFP, pol-I-PIC-GP-GFP;
(D) rPIC^{wt}: pC-PIC-L-Bsm, pC-PIC-NP-Bbs, pol-I-PIC-L, pol-I-PIC-S

We found GFP expression 48 hours after transfection of the S and L segment minigenomes (FIG. 4, plasmid combinations A and B as outlined above), documenting the intracellular reconstitution of functional Pichinde virus S and L segment analogues as ribonucleoproteins (RNPs), which were active in gene expression. Analogously, the transfection C aimed at generating r3PIC-GFP^{art} evidenced GFP-positive cells at 48 hours after transfection, whereas the plasmid combination D for generating rPIC^{wt} did not evidence green fluorescence, as expected. At 168 hours after transfection, GFP-positive cells had mostly disappeared in the S and L segment minigenome transfections, but were abundant in cells with r3PIC-GFP^{art}, indicating that an infectious, GFP-expressing virus had been reconstituted from cDNA and spread in the cell culture

5.2.3 Recombinant Tri-Segmented Viruses Grow to Lower Titers than Wild-Type Pichinde Virus

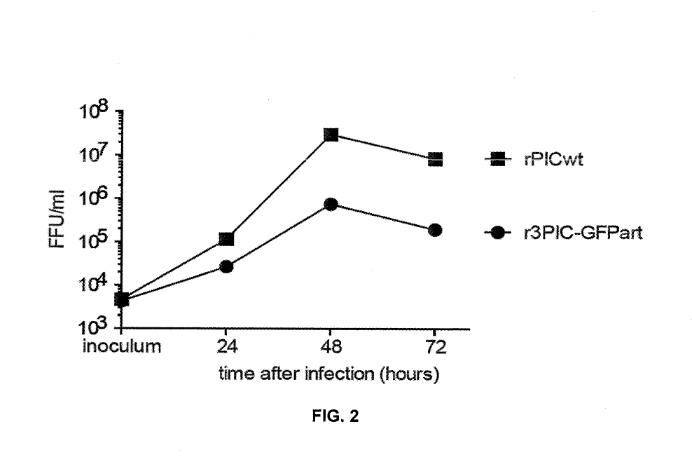

Comparative growth curves were performed with the viruses obtained with rPIC^{wt} and r3PIC-GFP^{art} (FIG. 2). Supernatant from transfections C and D from section 5.2.2 were collected and passaged in parallel in BHK-21 cells (multiplicity of infection=0.01, FIG. 2). For both viruses, peak infectivity was reached after 48 hours, yet for r3PIC-GFP^{art} was substantially lower than for rPIC. This indicated that the trisegmented r3PIC-GFP^{art} was attenuated as compared to its bisegmented wild type parental virus.

5.2.4 Recombinant r3PIC Expressing sP1AGM Induces a Rapid, Strong and Polyfunctional PIA-Specific CD8+ T Cell Response.

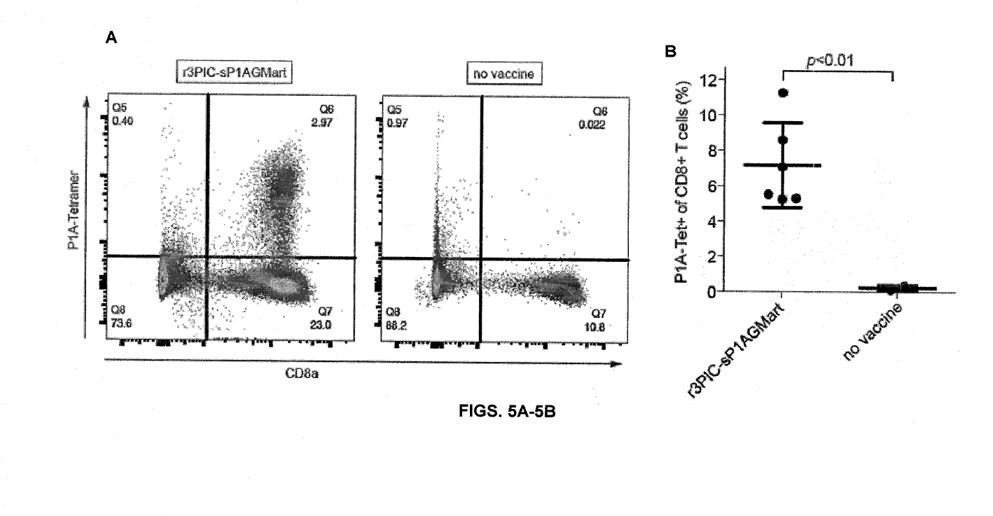

To test the utility of the r3PIC^{art} vector delivery technology for vaccination purposes we generated the r3PIC-sP1AGM^{art} vaccine vector (FIG. 1D) with a genome organization analogous to r3PIC-GFP^{art} (FIG. 1C). We created a virus expressing sP1AGM (r3PIC-sP1AGM^{art}), by procedures analogous to those outlined above for r3PIC-GFP^{art}, but using plasmids pol-I-PIC-NP-sP1AGM and pol-I-PIC-GP-sP1AGM instead of pol-I-PIC-NP-GFP and pol-I-PIC-GP-GFP, respectively. We immunized BALB/c mice intravenously with 10e5 focus forming units (FFU) r3PIC-sP1AGM^{art} i.v. and eight days later measured CD8+ T cell responses against the immunodominant P1A-derived H-2Ld-restricted epitope LPYLGWLVF (Aa35-43) by flow cytometry using MHC class I tetramers. r3PIC-sP1AGM^{art}-immunized mice exhibited very substantial populations of P1A35-43-specific CD8 T cells in peripheral blood, which were absent from the blood of unimmunized mice (FIGS. 5A and 5B). These observations demonstrated that r3PIC-art-based viral vectors are highly immunogenic, rendering them promising tools for immunotherapy and vaccination.

5.2.5 when Tested in an Early Passage after Rescue from cDNA, Both a Recombinant Tri-Segmented Virus Designed to Express its Glycoprotein and Nucleoprotein Genes in their Respective Natural Position and Also a Recombinant Tri-Segmented Virus Artificially Designed to Express its Glycoprotein Under Control of its 3′ Untranslated Region (UTR) Promoter Grow to Lower Titers than Wild-Type Pichinde Virus

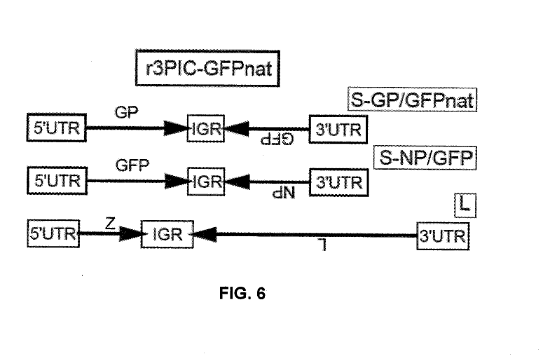

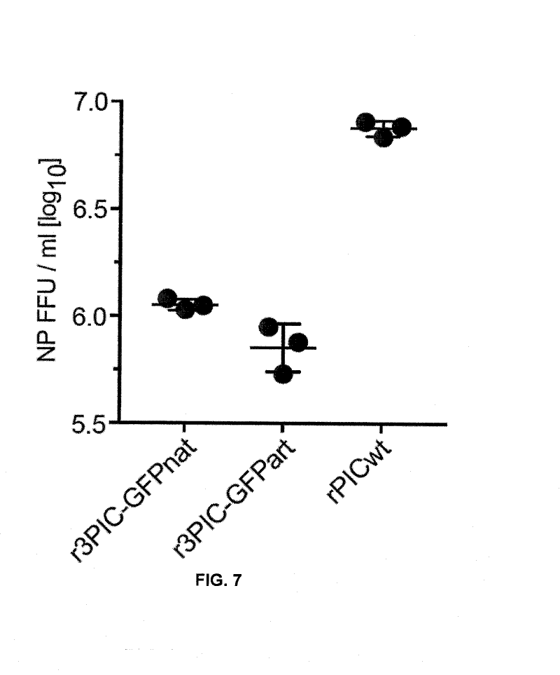

We generated a trisegmented Pichinde virus that expressed its glycoprotein (GP) and nucleoprotein (NP) genes under control of the 5′ and 3′ UTR promoters, respectively, i.e. in their respective “natural” position in the context of artificially duplicated S segments consisting of S-GP/GFPnat (SEQ ID NO: 15) and S-NP/GFP (also known as PIC-NP-GFP; SEQ ID NO: 11) (FIG. 6). This r3PIC-GFP^{nat} virus was created by procedures analogous to those outlined above for the trisegmented r3PIC-GFP^{art} virus. r3PIC-GFP^{nat} expressed GFP as schematically outlined in FIG. 6. When grown in BHK-21 cells in culture (multiplicity of infection=0.01, harvested at 48 hours), r3PIC-GFP^{nat} reached substantially lower titers than rPIC, titers that were similarly low as those observed for r3PIC-GFP^{art} (FIG. 7; symbols show titers from individual parallel cell culture wells; error bars denote the mean+/−SD). This indicated that the trisegmented r3PIC-GFP^{nat} was attenuated as compared to its bisegmented wild type parental virus.

5.2.6 During Persistent Infection of Immunodeficient Mice, Recombinant Tri-Segmented Viruses with an Artificial Genome Organization (r3PIC-GFP^{art}) Retain Transgenic GFP Expression and Remain at Consistently Lower Viral Titers in Blood than Wild-Type Pichinde Virus (rPIC^{wt}) Whereas Tri-Segmented Virus Designed to Express its Glycoprotein and Nucleoprotein Genes in their Respective Natural Position (r3PIC-GFP^{nat}) Eventually Lose GFP Expression and Reach Viral Loads in Blood Equivalent to Animals Infected with rPIC^{wt}.

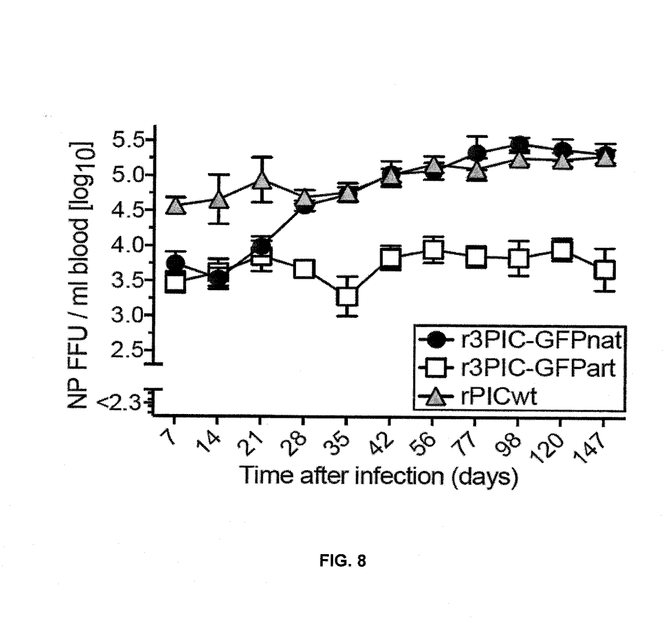

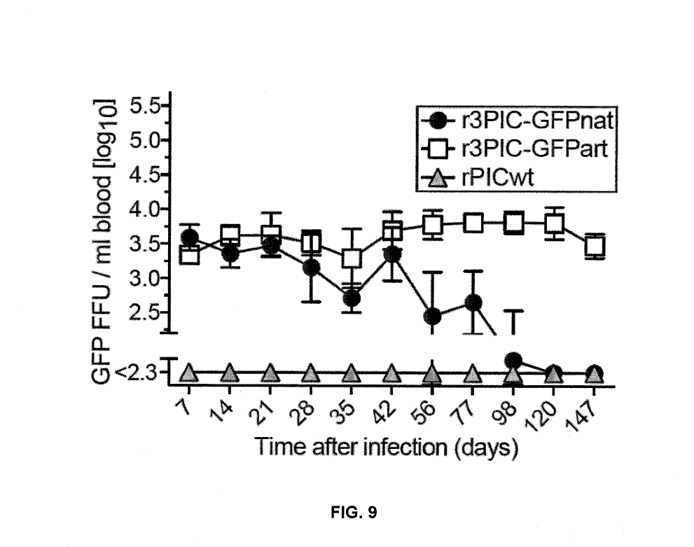

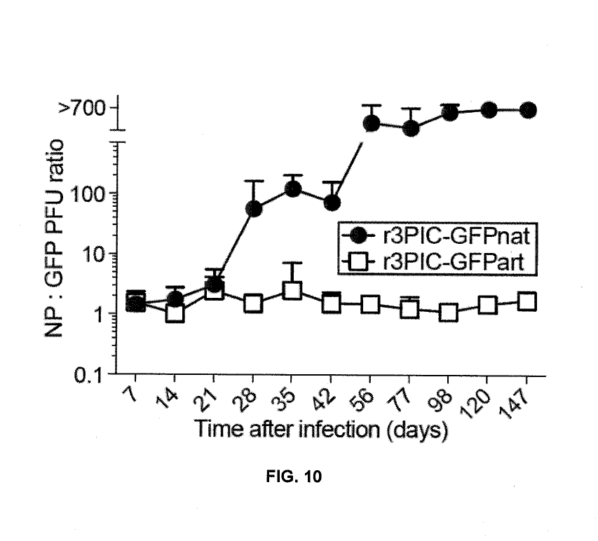

We infected mice triple-deficient in type I and type II interferon receptors as well as RAG1 (AGR mice; Grob et al, 1999, Role of the individual interferon systems and specific immunity in mice in controlling systemic dissemination of attenuated pseudorabies virus infection. J Virol, 4748-54) with 10e5 focus-forming units (“FFU”) of either one of r3PIC-GFP^{art}, r3PIC-GFP^{nat}, or rPIC^{wt} viruses intravenously (i.v.) on day 0. We collected blood on day 7, 14, 21, 28, 35, 42, 56, 77, 98, 120 and 147 and determined viral infectivity by FFU assays. In these assays we detected either the Pichinde virus nucleoprotein (NP FFU; FIG. 8) or the viral GFP transgenes in r3PIC-GFP^{nat} and r3PIC-GFP (GFP FFU; FIG. 9). From these values we calculated for each animal and time point the NP: GFP FFU ratio (FIG. 10).

During the first 21 days after infection, r3PIC-GFP^{nat} and r3PIC-GFP total infectivity (as determined by NP FFU assay) persisted at similar levels in the blood of AGR mice and was approximately ten-fold lower than in rPIC^{wt}-infected controls (FIG. 8). From day 28 onwards, however, r3PIC-GFP^{nat} infectivity, as determined by NP FFU assay, reached levels that were indistinguishable from rPIC. Conversely, r3PIC-GFP NP FFU titers remained at approximately 10-fold lower levels than those of rPICwt throughout the observation period of 147 days (FIG. 8).

Besides detecting the viral structural protein NP for determining the total viral infectivity (FIG. 8), we also performed FFU assays to assess GFP-expressing transgene-expressing infectivity in the blood of r3PIC-GFP^{nat}- and r3PIC-GFP^{art}-infected AGR mice (GFP FFU, FIG. 9). In striking contrast to NP FFU titers (FIG. 8), GFP FFU titers in r3PIC-GFP^{nat}-infected AGR mice dropped from day 28 onwards and were undetectable from day 120 onwards (FIG. 9). This contrasted with largely constant GFP FFU titers in the blood of r3PIC-GFP-infected mice (FIG. 9). By calculating the “NP: GFP FFU ratio” (FIG. 10), we determined that in r3PIC-GFP^{art}-infected mice, virtually all infectivity (NP FFU) expressed also the GFP transgene. This was borne out in a “NP:GFP FFU ratio” in the range of 1 throughout the observation period of 147 days (FIG. 10). In stark contrast, “NP: GFP FFU ratios” in the blood of r3PIC-GFP^{nat}-infected mice also started out around 1 but reached into the hundreds and above from day 28 onwards (FIG. 10). This indicated that within the population of virions circulating in the blood of r3PIC-GFPnat-infected mice on day 28 and thereafter only about one in one hundred or less still expressed the GFP transgene, and that GFP-expressing infectivity dropped eventually to below detectable levels. Hence, r3PIC-GFP retained GFP transgene expression throughout 147 days of persistent infection in AGR mice.

5.2.7 Viruses Recovered from the Serum of r3PIC-GFP^{art}-Infected Mice Remained Attenuated as Compared to Those from r3PIC-GFP^{nat}-Infected Animals, which Reached Titers Similar to Virus Isolated from r3PIC^{wt}-Infected Animals

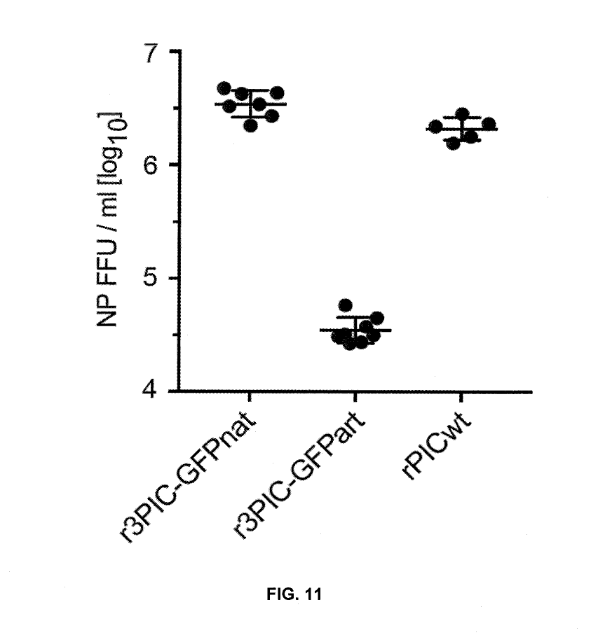

To assess the growth properties of viruses circulating in the serum of persistently infected AGR mice, we passaged viremic serum collected on day 147 after infection on BHK-21 cells and determined viral infectivity by NP FFU assays 48 hours later. The viruses grown from the serum of r3PIC-GFP^{nat}-infected mice reached IFF titers similar or higher than those from rPIC^{wt} virus-infected animals (FIG. 11; symbols show titers of individual mouse serum-derived viruses; error bars denote the mean+/−SD). Conversely, viral titers obtained after passage of serum from r3PIC-GFP^{art}-infected mice were substantially lower than either one of the aforementioned groups (FIG. 11).

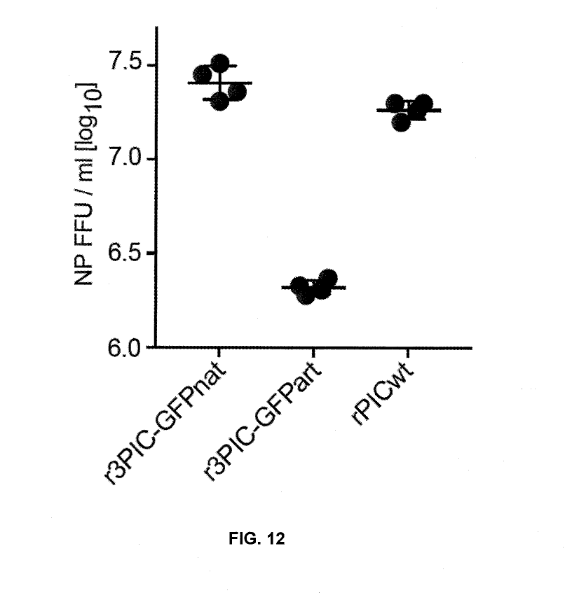

From these viruses, which had been passaged for 48 hours, we randomly chose four from each group for further analysis of cell culture growth. Unlike the experiment displayed in FIG. 11 (direct passage of infectivity from serum), this experiment (FIG. 12) was normalized for input infectivity and thereby excluded differential amounts of input infectivity as a potential confounder in the assessment of viral titers reached in culture. Accordingly, we infected BHK-21 cells at a standardized multiplicity of infection=0.01 and determined viral titers 48 hours later (FIG. 12; symbols show titers from individual mouse serum-derived viruses; error bars denote the mean+/−SD). Analogously to the differences in titers found after direct ex vivo passage from serum, r3PIC-GFP^{nat}-derived viruses reached titers that were at least equivalent to those of rPIC^{wt}-derived viruses. Conversely, the titers reached by viruses derived from in vivo passaged r3PIC-GFP^{art} were substantially lower than those of the aforementioned two groups.

This suggested that the virus recovered from the serum of r3PIC-GFP^{nat}-infected animals was no longer attenuated while the virus circulating in the blood of r3PIC-GFP^{art}-infected mice was still clearly attenuated as compared to rPIC^{wt}-derived viruses. Hence, as judged from lower r3PIC-GFP^{art} viremia than rPIC^{wt} viremia throughout the experiment in AGR mice (see section 5.2.6), and also from lower r3PIC-GFP^{art} titers than rPIC^{wt} titers when re-amplified from blood in cell culture, r3PIC-GFP^{art} retained its attenuation throughout the 147 day-period of in vivo replication in mice.

5.2.8 Unlike r3PIC-GFP^{nat}, Recombinant Tri-Segmented Virus with an Artificial Genome Organization (r3PIC-GFP^{art}) Did not Recombine its Two S Segments and Retained its Transgenes

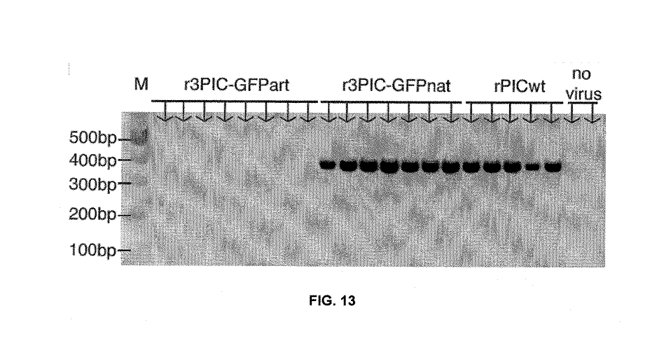

We wanted to determine whether in the course of persistent infection in AGR mice, r3PIC-GFP^{nat} may have recombined its two S segments to reunite the NP and GP genes on a single RNA segment, thereby eliminating the GFP transgenes. To test this possibility, we extracted viral RNA from serum samples collected from each animal on day 147 after viral infection. We performed RT-PCR using primers that were designed to bind to Pichinde virus NP and GP, respectively, and that spanned the intergenic region (“IGR”) of the Pichinde virus S segment such that they were predicted to yield a PCR amplicon of 357 base pairs on the rPIC^{wt} genome template. Such amplicons were indeed obtained when using viral RNA from the animals infected with either rPIC^{wt} or r3PIC-GFP^{nat}, but not when using viral RNA from the blood of r3PIC-GFP^{art}-infected mice (FIG. 13; each lane represents the RT-PCR product from one individual mouse in the experiment shown in FIGS. 8-10).

Taken together, these data indicated that in the course of persistent infection of AGR mice, r3PIC-GFP^{nat} recombined its two S segments (S-GP/GFPnat, S-NP/GFP) to re-unite the NP and GP open reading frames in one single segment of RNA. Thereby it lost expression of the GFP transgenes and augmented its growth capacity to the one of rPIC^{wt}, both in mice as evident in the levels of viremia and in cell culture as seen upon harvest from blood and re-expansion in cell culture. Conversely, r3PIC-GFP^{art} failed to recombine its two S segments as evident in the lack of an RT-PCR amplicon spanning the NP and GP genes.

### 6. EQUIVALENTS

The viruses, nucleic acids, methods, host cells, and compositions disclosed herein are not to be limited in scope by the specific embodiments described herein. Indeed, various modifications of the viruses, nucleic acids, methods, host cells, and compositions in addition to those described will become apparent to those skilled in the art from the foregoing description and accompanying figures. Such modifications are intended to fall within the scope of the appended claims.

Various publications, patents and patent applications are cited herein, the disclosures of which are incorporated by reference in their entireties.

Various publications, patents and patent applications are cited herein, the disclosures of which are incorporated by reference in their entireties.

7. SEQUENCE LISTING

| SEQ ID | | |
|---|---|---|
| NO. | Description | Sequence |
| 1 | PIC-S: Pichinde | gcgcaccggg gatcctaggc ataccttgga |
| | virus strain | cgcgcatatt acttgatcaa agatgggaca |
| | Munchique CoAn4763 | agttgtgact ttgatccagt ctatacccga |
| | isolate P18 (Genbank | agtcctgcag gaggtcttca atgtcgcctt |
| | accession number | aatcattgtc tcaaccctat gcatcatcaa |
| | EF529746.1) segment | aggatttgtc aatctgatga gatgtggcct |
| | S, complete | attccaactc atcaccttcc tcattttggc |
| | sequence. The | tggcagaagt tgtgatggca tgatgattga |
| | genomic segment is | taggaggcac aatctcaccc acgttgagtt |
| | RNA, the sequence in | caacctcaca agaatgtttg acaacttgcc |
| | SEQ ID NO: 1 is | acaatcatgt agcaagaaca acacacatca |
| | shown for DNA; | ttactacaaa ggaccatcta acacaacatg |
| | however, exchanging | gggaattgaa ctcactttga caaacacatc |
| | all thymidines (“T”) | cattgcaaat gaaactactg gaaacttttc |
| | in SEQ ID NO: 1 for | caacatcaga agccttgcat atggtaacat |
| | uridines (“U”) | tagtaattgt gataagacag aagaagcagg |
| | provides the RNA | tcacacatta aaatggttgc ttaatgagtt |
| | sequence. | acacttcaat gtgctccatg tcactcgtca |
| | | tgtaggtgcc agatgcaaaa cagttgaggg |
| | | tgctggggtg ttgatccagt acaacttgac |
| | | agttggggat agaggaggtg aggttggcag |
| | | acatcttatt gcgtcgcttg ctcaaatcat |
| | | tggggaccca aaaattgcgt gggttggaaa |
| | | atgtttcaat aactgtagtg gagggtcttg |
| | | cagactaaca aactgtgaag gtgggacaca |
| | | ttacaatttc ctgatcatac agaacaccac |
| | | atgggaaaat cactgtacat atactccaat |
| | | ggcaacaata aggatggctc tccaaaaaac |
| | | tgcttatagt tctgtgagca ggaaactcct |
| | | tggctttttc acttgggact tgagtgactc |
| | | tactgggcaa catgtcccag gtggttactg |
| | | tttggagcaa tgggctattg tttgggctgg |
| | | aataaaatgt tttgataaca ctgtgatggc |
| | | aaaatgcaac aaagatcaca atgaagaatt |
| | | ttgcgatacg atgaggttat ttgatttcaa |
| | | tcagaatgct atcaaaacct tacaacttaa |
| | | tgttgagaat tcgttgaatc tctttaaaaa |
| | | gactatcaac ggacttattt ctgactcact |
| | | tgtgattaga aacagtctca aacagcttgc |
| | | caaaatccct tattgcaact atacaaaatt |
| | | ttggtacatc aatgatacca tcacaggaag |
| | | acattcttta ccgcagtgtt ggttagttca |
| | | caatggctcg tacctcaatg aaacgcattt |
| | | taagaatgat tggttgtggg agagccagaa |
| | | tctgtacaat gaaatgctga taaaagaata |
| | | tgaagaaaga caaggtaaga ctccactagc |
| | | attgacagac atttgcttct ggtctttggt |
| | | gttttacacc atcacagtgt ttctccactt |
| | | agttggaata cccactcata ggcacatcat |
| | | tggtgatggc tgtccgaagc cacataggat |
| | | tactaggaac tctctttgca gctgtgggta |
| | | ttataaaatc ccaaagaaac cctacaaatg |
| | | ggtgagactg ggtaaataag ccctagcctc |
| | | gacatgggcc tcgacgtcac tccccaatag |
| | | gggagtgacg tcgaggcctc tgaggacttg |
| | | agctcagagg ttgatcagat ctgtgttgtt |
| | | cctgtacagc gtgtcaatag gcaagcatct |
| | | catcggcttc tggtccctaa cccagcctgt |
| | | cactgttgca tcaaacatga tggtatcaag |
| | | caatgcacag tgaggattcg cagtggtttg |
| | | tgcagccccc ttcttcttct tctttatgac |
| | | caaaccttta tgtttggtgc agagtagatt |
| | | gtatctctcc cagatctcat cctcaaaggt |
| | | gcgtgcttgc tcggcactga gtttcacgtc |
| | | aagcactttt aagtctcttc tcccatgcat |
| | | ttcgaacaaa ctgattatat catctgaacc |
| | | ttgagcagtg aaaaccatgt tttgaggtaa |
| | | atgtctgatg attgaggaaa tcaggcctgg |
| | | ttgggcatca gccaagtcct ttaaaagaag |
| | | accatgtgag tacttgcttt gctctttgaa |
| | | ggacttctca tcgtggggaa atctgtaaca |
| | | atgtatgtag ttgcccgtgt caggctggta |
| | | gatggccatt tccaccggat catttggtgt |
| | | tccttcaatg tcaatccatg tggtagcttt |
| | | tgaatcaagc atctgaattg aggacacaac |
| | | agtgtcttct ttctccttag ggatttgttt |
| | | aaggtccggt gatcctccgt ttcttactgg |
| | | tggctggata gcactcggct tcgaatctaa |
| | | atctacagtg gtgttatccc aagccctccc |
| | | ttgaacttga gaccttgagc caatgtaagg |
| | | ccaaccatcc cctgaaagac aaatcttgta |
| | | tagtaaattt tcataaggat ttctctgtcc |
| | | gggtgtagtg ctcacaaaca taccttcacg |
| | | attctttatt tgcaatagac tctttatgag |
| | | agtactaaac atagaaggct tcacctggat |
| | | ggtctcaagc atattgccac catcaatcat |
| | | gcaagcagct gctttgactg ctgcagacaa |
| | | actgagattg taccctgaga tgtttatggc |
| | | tgatggctca ttactaatga tttttagggc |
| | | actgtgttgc tgtgtgagtt tctctagatc |
| | | tgtcatgttc gggaacttga cagtgtagag |
| | | caaaccaagt gcactcagcg cttggacaac |
| | | atcattaagt tgttcacccc cttgctcagt |
| | | catacaagcg atggttaagg ctggcattga |
| | | tccaaattga ttgatcaaca atgtattatc |
| | | cttgatgtcc cagatcttca caaccccatc |
| | | tctgttgcct gtgggtctag cattagcgaa |
| | | ccccattgag cgaaggattt cggctctttg |
| | | ttccaactga gtgtttgtga gattgccccc |
| | | ataaacacca ggctgagaca aactctcagt |
| | | tctagtgact ttctttctta acttgtccaa |
| | | atcagatgca agctccatta gctcctcttt |
| | | ggctaagcct cccaccttaa gcacattgtc |
| | | cctctggatt gatctcatat tcatcagagc |
| | | atcaacctct ttgttcatgt ctcttaactt |
| | | ggtcagatca gaatcagtcc ttttatcttt |
| | | gcgcatcatt ctttgaactt gagcaacttt |
| | | gtgaaagtca agagcagata acagtgctct |
| | | tgtgtccgac aacacatcag ccttcacagg |
| | | atgggtccag ttggatagac ccctcctaag |
| | | ggactgtacc cagcggaatg atgggatgtt |
| | | gtcagacatt ttggggttgt ttgcacttcc |
| | | tccgagtcag tgaagaagtg aacgtacagc |
| | | gtgatctaga atcgcctagg atccactgtg cg |
| 2 | PIC-L-seg: Pichinde | gcgcaccggg gatcctaggc atctttgggt |
| | virus strain | cacgcttcaa atttgtccaa tttgaaccca |
| | Munchique CoAn4763 | gctcaagtcc tggtcaaaac ttgggatggg |
| | isolate P18 (Genbank | actcagatat agcaaagagg tcaggaagag |
| | accession number | acatggcgac gaagatgtgg tgggaagggt |
| | EF529747.1) segment | ccccatgacc ctcaatctac cacagggcct |
| | L, complete sequence | gtatggcagg ttcaactgca aatcttgctg |
| | with non-coding | gttcgtcaac aaaggtctca tcaggtgcaa |
| | mutations introduced | agaccactat ctgtgtcttg ggtgcttaac |
| | to delete BsmBI | caaaatgcac tccagaggca atctctgcga |
| | restriction sites. | gatatgcggc cactcactgc caaccaagat |
| | The genomic segment | ggagttccta gaaagcccct ctgcaccacc |
| | is RNA; however, | ctacgagcca taaaccaggg cccctgggcg |
| | exchanging all | cacccccctc cgggggtgcg cccgggggcc |
| | thymidines (“T”) in | cccggcccca tggggccggt tgtttactcg |
| | SEQ ID NO: 4 for | atctccactg actcattgtc ctcaaacaac |
| | uridines (“U”) | tttcgacacc tgattccctt gatcttgaag |
| | provides the RNA | ggtcctgtct cgtctgcaat cataacagat |
| | sequence. | cctagagtct tacttcttat tatactaaag |
| | | tgaccacaat tcaaccaatc tttggcatca |
| | | tgcaacatgt gttcaaacac ttcggggaaa |
| | | ttttcaatca tgagtcttaa atcctgctcg |
| | | ttcatactta ttcccttgtt gtgagactgt |
| | | gcacttgaaa ggtactgaaa aaggttggca |
| | | ataaatcttg gccttttctc aggttctaat |
| | | gcttccagtg caatgatgac cacctttgag |
| | | tctaagttca cttccaatct agaaaccact |
| | | ctgttgccct ctttgatcaa cccaccctct |
| | | aaaatgaggg gttgcatccc aacatcagga |
| | | ccaatcaact tataggaaaa tttgtttttc |
| | | aaatccttga aacgattttt caaatctatt |
| | | ctcaccttct ggaacacagt tgaccttgac |
| | | ttgaagtgaa tgtcttgacc ttccaataga |
| | | tcattgaagt ctagaacatc ttttccgttg |
| | | atgagaggat tcagaaccaa aagtgacaca |
| | | ccatccagac ttatgtgatt cccggaagat |
| | | tgagaaacat aatactcaac agaatggggg |
| | | ttcaacaata ggtaaccatc agagtccaat |
| | | gagtccagca atgactccct ttcaataaga |
| | | aatcttaatt ttaatatgta attggtagac |
| | | ctctcatatc taaatttgtg gctcactctc |
| | | ttatgagaaa atgttaggtt gagctcaatg |
| | | ggaatgacct cagaaggtga tgctaaaatg |
| | | agttgttcaa tgttctcata gttatctcta |
| | | ttcacccagt caagttcatt aataaataca |
| | | ctaatgttca aattaacaca ggacaaaatc |
| | | agtttgctgc ttacaaagcc aacatccaag |
| | | tcatccagat tcattgtcct agaagtgtta |
| | | ttctttttgc agtcacaaat gaactgggtt |
| | | aattgtttca gatcatgttg tgcattgttt |
| | | ggcaacaatt caagctcacc aaaccaaaaa |
| | | tatttcttga actgagatgt tgacataatc |
| | | acaggcacca acattgactc aaacaaaatc |
| | | tgtatcaaga aatttgtgca cacttcttct |
| | | ggttcaaggt tgaatcctct ctccagtgga |
| | | tgagactctc tgctatggga cattgcaagc |
| | | tcattttgct ttacaatata caattcttct |
| | | ctgcgatgtt ttataatatg actaacaata |
| | | ccaagacatt ctgatgttat atcaattgcc |
| | | acacaaaggt ctaagaactt tatcctctga |
| | | acccatgata gcctcagcat attcaaatca |
| | | gacaggaaag gggatatgtg ttcatcaaat |
| | | agtgtaggga agttcctcct gattgagtaa |
| | | agtatgtggt tgatgcccac cttgtcctca |
| | | agctcagaat gtgtgcttgg ttttattggc |
| | | cagaagtgat tgggattgtt taggtgagtg |
| | | actatcttgg gtacttcagc tttttgaaac |
| | | acccagttac ccaactcgca agcattggtt |
| | | aacacaagag caaaataatc ccaaattaag |
| | | ggtctggagt actcacttac ttcaccaagt |
| | | gctgctttac aataaacacc tttgcgctga |
| | | ttacaaaagt gacaatcacg gtgtaagata |
| | | atcttgcttg taatatccct gatatactta |
| | | aatcctcctt tcccatctct tacacatttt |
| | | gagcccatac ttttgcaaac tcctatgaat |
| | | cctgatgcta tgctgctctg aaaagctgat |
| | | ttgttgatag catcagccaa aatcttctta |
| | | gcccctctga catagttctt tgataatttg |
| | | gactgtacgg atttgacaag actgggtatt |
| | | tcttctcgct gcacagttct tgttgtgctc |
| | | attaacttag tacgaagcac caatctgaga |
| | | tcaccatgaa cccttaaatt taaccaccta |
| | | atattaagag catcctcaat agcctcagtc |
| | | tcgacatcac aagtctctaa taactgtttt |
| | | aagcagtcat ccggtgattg ctgaagagtt |
| | | gttacaatat aactttcttc cagggctcca |
| | | gactgtattt tgtaaaatat tttcctgcat |
| | | gcctttctga ttattgaaag tagcagatca |
| | | tcaggaaata gtgtctcaat tgatcgctga |
| | | agtctgtacc ctctcgaccc attaacccaa |
| | | tcgagtacat ccatttcttc caggcacaaa |
| | | aatggatcat ttggaaaccc actatagatt |
| | | atcatgctat ttgttcgttt tgcaatggcc |
| | | cctacaacct ctattgacac cccgttagca |
| | | acacattggt ccagtattgt gtcaattgta |
| | | tctgcttgct gattgggtgc tttagccttt |
| | | atgttgtgta gagctgcagc aacaaacttt |
| | | gtaaggaggg ggacttcttg tgaccaaatg |
| | | aagaatctcg atttgaactc acttgcaaag |
| | | gtccccacaa ctgttttagg gctcacaaac |
| | | ttgttgagtt tgtctgatag aaagtagtga |
| | | aactccatac agtccaatac caattcaaca |
| | | ttcaactcat ctctgtcctt aaatttgaaa |
| | | ccctcattca aggataacat gatctcatca |
| | | tcactcgaag tatatgagat gaaccgtgct |
| | | ccataacaaa gctccaatgc gtaattgatg |
| | | aactgctcag tgattagacc atataagtca |
| | | gaggtgttgt gtaggatgcc ctgacccata |
| | | tctaagactg aagagatgtg tgatggtacc |
| | | ttgcccttct caaagtaccc aaacataaat |
| | | tcctctgcaa ttgtgcaccc ccctttatcc |
| | | atcataccca accccctttt caagaaacct |
| | | ttcatgtatg cctcaacgac attgaagggc |
| | | acttccacca tcttgtgaat gtgccatagc |
| | | aatatgttga tgactgcagc attgggaact |
| | | tctgacccat ctttgagttt gaactcaaga |
| | | ccttttaata atgcggcaaa gataaccggc |
| | | gacatgtgtg gcccccattt tgaatggtcc |
| | | attgacaccg caagaccact ttgcctaaca |
| | | actgacttca tgtctaataa tgctctctca |
| | | aactctttct cgttgttcag acaagtatac |
| | | ctcatgtttt gcataaggga ttcagagtaa |
| | | tcctcaatga gtctggttgt gagtttagta |
| | | tttaaatcac cgacataaag ctccctgttg |
| | | ccacccacct gttctttata agaaagacca |
| | | aatttcaatc tccctacatt ggtggataca |
| | | ccagacctct ctgtgggaga ctcatctgaa |
| | | tagaaacaga gatttcgtaa ggatgagttg |
| | | gtaaaaaagc tttgatccaa tcttttagct |
| | | atcgattcag aattgctctc tcttgagctt |
| | | atacgtgatg tctctctaat ttgtagtgct |
| | | gcatctgtga acccaagtct gcttctactt |
| | | ttgtgatcat atcttccgac tcgattatca |
| | | taatcgcttg caatgagaat gtatttaaag |
| | | cactcaaaat aatcagcttc tttgtacgcc |
| | | ttcaatgtga ggttctttat taaaaactcc |
| | | agaggacacg gattcattag tctgtctgca |
| | | aagtacactg atctagcagt gacatcctca |
| | | tagatcaagt ttacaagatc ctcatacact |
| | | tctgctgaaa acaggctgta atcaaaatcc |
| | | tttacatcat gaagtgaagt ctctcttttg |
| | | atgacaacca ttgtcgattt gggccataat |
| | | ctctctagtg gacatgaagt cttaaggttg |
| | | gttttgacat tggtgtcaac cttagacaat |
| | | acttttgcaa ctctggtctc aatttcttta |
| | | agacagtcac cctgatcttc tgatagtaac |
| | | tcttcaactc catcaggctc tattgactcc |
| | | ttttttattt ggatcaatga tgacaacctc |
| | | ttcagaatct tgaaatttac ctcctttgga |
| | | tctaacttgt atttaccctt agttttgaaa |
| | | tgttcaatca tttccacaac aacagcagac |
| | | acaatggaag agtaatcata ttcagtgatg |
| | | acctcaccaa cttcattgag ttttggaacc |
| | | accacacttt tgttgctgga catatccaag |
| | | gctgtacttg tgaaggaggg agtcataggg |
| | | tcacaaggaa gcaggggttt cacttccaat |
| | | gagctactgt taaatagtga tagacaaaca |
| | | ctaagtacat ccttattcaa ccccggcctt |
| | | ccctcacatt tggattccag ctttttacca |
| | | agtagtctct ctatatcatg caccatcttc |
| | | tcttcttcct cagtaggaag ttccatacta |
| | | ttagaagggt tgaccaagac tgaatcaaac |
| | | tttaactttg gttccaagaa cttctcaaaa |
| | | catttgattt gatcagttaa tctatcaggg |
| | | gtttctttgg ttataaaatg gcataaatag |
| | | gagacattca aaacaaactt aaagatctta |
| | | gccatatctt cctctctgga gttgctgagt |
| | | accagaagta tcaaatcatc aataagcatt |
| | | gctgtctgcc attctgaagg tgttagcata |
| | | acgactttca atttctcaaa caattcttta |
| | | aaatgaactt catttacaaa ggccataatg |
| | | taatatctaa agccttgcaa gtaaacttga |
| | | atacgcttgg aaggggtgca cagtatgcag |
| | | agaataagtc gtctgagtaa atcagaaaca |
| | | gaatccaaga ggggttggga cataaagtcc |
| | | aaccaggata acatctccac acaagtcctt |
| | | tgaatcacat ctgcactaaa gatcggtaag |
| | | aaaaatctct tgggatcaca gtaaaaagac |
| | | gcttttgttt catacaaacc cccacttttg |
| | | gatctataag caacagcata acacctggac |
| | | ctctcccctg tcttctggta cagtagtgtg |
| | | agagaacctc cttctccaaa tcgctggaag |
| | | aaaacttcgt cacagtaaac cttcccataa |
| | | aactcatcag cattgttcac cttcatctta |
| | | ggaactgctg ctgtcttcat gctattaatg |
| | | agtgacaaac tcaaacttga caatgttttc |
| | | agcaattcct caaactcact ttcgcccatg |
| | | atggtataat caggctgccc tcttcctggc |
| | | ctacccccac acatacactg tgactttgtc |
| | | ttgtattgaa gacagggttt agcaccccat |
| | | tcatctaaca ctgatgtttt cagattgaag |
| | | taatattcaa catcaggttc ccgtagaaga |
| | | gggagaatgt catcaagggg aagttcacca |
| | | cagaccgagc tcagtctctt cttagccttc |
| | | tctaaccagt tggggttttt aatgaatttt |
| | | ttagtgattt gttccatcag gaagtcgaca |
| | | ttaatcaacc tgtcatttac agacggtaac |
| | | ccttgcatta ggagcacctc tctgaacaca |
| | | gcacctggag aagacttgtc caagtcacac |
| | | aaaatgttgt acatgataag gtccagaacc |
| | | aacatggtgt tcctccttgt gttaaaaacc |
| | | ttttgagact taattttgtt gcatattgaa |
| | | agtactctaa aatattctct gctttcagtt |
| | | gatgaatgct tgacctcaga ttgcctgagt |
| | | tggcctatta tgcccaaaat gtgtactgag |
| | | caaaactcac ataatctgat ttctgattta |
| | | ggtacatctt tgacagaaca ttggataaat |
| | | tcatggttct gaagtctaga aatcatatct |
| | | tccctatctg tagcctgcag tttcctatcg |
| | | agttgaccag caagttgcaa cattttaaat |
| | | tgctgaaaga tttccatgat ttttgttcta |
| | | cattgatctg ttgtcagttt attattaatg |
| | | ccagacatta atgccttttc caacctcact |
| | | ttgtaaggaa gtcccctttc ctttacagca |
| | | agtagtgact ccagaccgag actctgattt |
| | | tctaaggatg agagggaact tataaggcgt |
| | | tcgtactcca actcctcaac ttcttcacca |
| | | gatgtcctta atccatccat gagttttaaa |
| | | agcaaccacc gaagtctctc taccacccaa |
| | | tcaggaacaa attctacata ataactggat |
| | | ctaccgtcaa taacaggtac taaggttatg |
| | | ttctgtctct tgagatcaga actaagctgc |
| | | aacagcttca aaaagtcctg gttgtatttc |
| | | ttctcaaatg cttcttgact ggtcctcaca |
| | | aacacttcca aaagaatgag gacatctcca |
| | | accatacagt aaccatctgg tgtaacatcc |
| | | ggcaatgtag gacatgttac tctcaactcc |
| | | ctaaggatag cattgacagt catctttgtg |
| | | ttgtgtttgc aggagtgttt cttgcatgaa |
| | | tccacttcca ctagcatgga caaaagcttc |
| | | aggccctcta tcgtgatggc cctatctttg |
| | | acttgtgcaa gaacgttgtt tttctgttca |
| | | gatagctctt cccattcggg aacccatttt |
| | | ctgactatgt ctttaagttc gaaaacgtat |
| | | tcctccatga tcaagaaatg cctaggatcc tcggtgcg |
| 3 | LΔBsmBI: | GAATTCcgtc tctgatcatg gaggaatacg |
| | Representative cDNA | ttttcgaact taaagacata gtcagaaaat |
| | of the L ORF of | gggttcccga atgggaagag ctatctgaac |
| | Pichinde virus | agaaaaacaa cgttcttgca caagtcaaag |
| | strain Munchique | atagggccat cacgatagag ggcctgaagc |
| | CoAn4763 isolate P18 | ttttgtccat gctagtggaa gtggattcat |
| | (Genbank accession | gcaagaaaca ctcctgcaaa cacaacacaa |
| | number EF529747.1), | agatgactgt caatgctatc cttagggagt |
| | wherein a non-coding | tgagagtaac atgtcctaca ttgccggatg |
| | mutation was | ttacaccaga tggttactgt atggttggag |
| | introduced to delete | atgtcctcat tcttttggaa gtgtttgtga |
| | the BsmBI | ggaccagtca agaagcattt gagaagaaat |
| | restriction site. | acaaccagga ctttttgaag ctgttgcagc |
| | ORF also contains | ttagttctga tctcaagaga cagaacataa |
| | flanking BsmBI | ccttagtacc tgttattgac ggtagatcca |
| | (bold) as well as | gttattatgt agaatttgtt cctgattggg |
| | EcoRI (uppercase)and | tggtagagag acttcggtgg ttgcttttaa |
| | NheI (uppercase and | aactcatgga tggattaagg acatctggtg |
| | italicized) | aagaagttga ggagttggag tacgaacgcc |
| | restriction sites. | ttataagttc cctctcatcc ttagaaaatc |
| | | agagtctcgg tctggagtca ctacttgctg |
| | | taaaggaaag gggacttcct tacaaagtga |
| | | ggttggaaaa ggcattaatg tctggcatta |
| | | ataataaact gacaacagat caatgtagaa |
| | | caaaaatcat ggaaatcttt cagcaattta |
| | | aaatgttgca acttgctggt caactcgata |
| | | ggaaactgca ggctacagat agggaagata |
| | | tgatttctag acttcagaac catgaattta |
| | | tccaatgttc tgtcaaagat gtacctaaat |
| | | cagaaatcag attatgtgag ttttgctcag |
| | | tacacatttt gggcataata ggccaactca |
| | | ggcaatctga ggtcaagcat tcatcaactg |
| | | aaagcagaga atattttaga gtactttcaa |
| | | tatgcaacaa aattaagtct caaaaggttt |
| | | ttaacacaag gaggaacacc atgttggttc |
| | | tggaccttat catgtacaac attttgtgtg |
| | | acttggacaa gtcttctcca ggtgctgtgt |
| | | tcagagaggt gctcctaatg caagggttac |
| | | cgtctgtaaa tgacaggttg attaatgtcg |
| | | acttcctgat ggaacaaatc actaaaaaat |
| | | tcattaaaaa ccccaactgg ttagagaagg |
| | | ctaagaagag actgagctcg gtctgtggtg |
| | | aacttcccct tgatgacatt ctccctcttc |
| | | tacgggaacc tgatgttgaa tattacttca |
| | | atctgaaaac atcagtgtta gatgaatggg |
| | | gtgctaaacc ctgtcttcaa tacaagacaa |
| | | agtcacagtg tatgtgtggg ggtaggccag |
| | | gaagagggca gcctgattat accatcatgg |
| | | gcgaaagtga gtttgaggaa ttgctgaaaa |
| | | cattgtcaag tttgagtttg tcactcatta |
| | | atagcatgaa gacagcagca gttcctaaga |
| | | tgaaggtgaa caatgctgat gagttttatg |
| | | ggaaggttta ctgtgacgaa gttttcttcc |
| | | agcgatttgg agaaggaggt tctctcacac |
| | | tactgtacca gaagacaggg gagaggtcca |
| | | ggtgttatgc tgttgcttat agatccaaaa |
| | | gtgggggttt gtatgaaaca aaagcgtctt |
| | | tttactgtga tcccaagaga tttttcttac |
| | | cgatctttag tgcagatgtg attcaaagga |
| | | cttgtgtgga gatgttatcc tggttggact |
| | | ttatgtccca acccctcttg gattctgttt |
| | | ctgatttact cagacgactt attctctgca |
| | | tactgtgcac cccttccaag cgtattcaag |
| | | tttacttgca aggctttaga tattacatta |
| | | tggcctttgt aaatgaagtt cattttaaag |
| | | aattgtttga gaaattgaaa gtcgttatgc |
| | | taacaccttc agaatggcag acagcaatgc |
| | | ttattgatga tttgatactt ctggtactca |
| | | gcaactccag agaggaagat atggctaaga |
| | | tctttaagtt tgttttgaat gtctcctatt |
| | | tatgccattt tataaccaaa gaaacccctg |
| | | atagattaac tgatcaaatc aaatgttttg |
| | | agaagttctt ggaaccaaag ttaaagtttg |
| | | attcagtctt ggtcaaccct tctaatagta |
| | | tggaacttcc tactgaggaa gaagagaaga |
| | | tggtgcatga tatagagaga ctacttggta |
| | | aaaagctgga atccaaatgt gagggaaggc |
| | | cggggttgaa taaggatgta cttagtgttt |
| | | gtctatcact atttaacagt agctcattgg |
| | | aagtgaaacc cctgcttcct tgtgacccta |
| | | tgactccctc cttcacaagt acagccttgg |
| | | atatgtccag caacaaaagt gtggtggttc |
| | | caaaactcaa tgaagttggt gaggtcatca |
| | | ctgaatatga ttactcttcc attgtgtctg |
| | | ctgttgttgt ggaaatgatt gaacatttca |
| | | aaactaaggg taaatacaag ttagatccaa |
| | | aggaggtaaa tttcaagatt ctgaagaggt |
| | | tgtcatcatt gatccaaata aaaaaggagt |
| | | caatagagcc tgatggagtt gaagagttac |
| | | tatcagaaga tcagggtgac tgtcttaaag |
| | | aaattgagac cagagttgca aaagtattgt |
| | | ctaaggttga caccaatgtc aaaaccaacc |
| | | ttaagacttc atgtccacta gagagattat |
| | | ggcccaaatc gacaatggtt gtcatcaaaa |
| | | gagagacttc acttcatgat gtaaaggatt |
| | | ttgattacag cctgttttca gcagaagtgt |
| | | atgaggatct tgtaaacttg atctatgagg |
| | | atgtcactgc tagatcagtg tactttgcag |
| | | acagactaat gaatccgtgt cctctggagt |
| | | ttttaataaa gaacctcaca ttgaaggcgt |
| | | acaaagaagc tgattatttt gagtgcttta |
| | | aatacattct cattgcaagc gattatgata |
| | | atcgagtcgg aagatatgat cacaaaagta |
| | | gaagcagact tgggttcaca gatgcagcac |
| | | tacaaattag agagacatca cgtataagct |
| | | caagagagag caattctgaa tcgatagcta |
| | | aaagattgga tcaaagcttt tttaccaact |
| | | catccttacg aaatctctgt ttctattcag |
| | | atgagtctcc cacagagagg tctggtgtat |
| | | ccaccaatgt agggagattg aaatttggtc |
| | | tttcttataa agaacaggtg ggtggcaaca |
| | | gggagcttta tgtcggtgat ttaaatacta |
| | | aactcacaac cagactcatt gaggattact |
| | | ctgaatccct tatgcaaaac atgaggtata |
| | | cttgtctgaa caacgagaaa gagtttgaga |
| | | gagcattatt agacatgaag tcagttgtta |
| | | ggcaaagtgg tcttgcggtg tcaatggacc |
| | | attcaaaatg ggggccacac atgtcgccgg |
| | | ttatctttgc cgcattatta aaaggtcttg |
| | | agttcaaact caaagatggg tcagaagttc |
| | | ccaatgctgc agtcatcaac atattgctat |
| | | ggcacattca caagatggtg gaagtgccct |
| | | tcaatgtcgt tgaggcatac atgaaaggtt |
| | | tcttgaaaag ggggttgggt atgatggata |
| | | aaggggggtg cacaattgca gaggaattta |
| | | tgtttgggta ctttgagaag ggcaaggtac |
| | | catcacacat ctcttcagtc ttagatatgg |
| | | gtcagggcat cctacacaac acctctgact |
| | | tatatggtct aatcactgag cagttcatca |
| | | attacgcatt ggagctttgt tatggagcac |
| | | ggttcatctc atatacttcg agtgatgatg |
| | | agatcatgtt atccttgaat gagggtttca |
| | | aatttaagga cagagatgag ttgaatgttg |
| | | aattggtatt ggactgtatg gagtttcact |
| | | actttctatc agacaaactc aacaagtttg |
| | | tgagccctaa aacagttgtg gggacctttg |
| | | caagtgagtt caaatcgaga ttcttcattt |
| | | ggtcacaaga agtccccctc cttacaaagt |
| | | ttgttgctgc agctctacac aacataaagg |
| | | ctaaagcacc caatcagcaa gcagatacaa |
| | | ttgacacaat actggaccaa tgtgttgcta |
| | | acggggtgtc aatagaggtt gtaggggcca |
| | | ttgcaaaacg aacaaatagc atgataatct |
| | | atagtgggtt tccaaatgat ccatttttgt |
| | | gcctggaaga aatggatgta ctcgattggg |
| | | ttaatgggtc gagagggtac agacttcagc |
| | | gatcaattga gacactattt cctgatgatc |
| | | tgctactttc aataatcaga aaggcatgca |
| | | ggaaaatatt ttacaaaata cagtctggag |
| | | ccctggaaga aagttatatt gtaacaactc |
| | | ttcagcaatc accggatgac tgcttaaaac |
| | | agttattaga gacttgtgat gtcgagactg |
| | | aggctattga ggatgctctt aatattaggt |
| | | ggttaaattt aagggttcat ggtgatctca |
| | | gattggtgct tcgtactaag ttaatgagca |
| | | caacaagaac tgtgcagcga gaagaaatac |
| | | ccagtcttgt caaatccgta cagtccaaat |
| | | tatcaaagaa ctatgtcaga ggggctaaga |
| | | agattttggc tgatgctatc aacaaatcag |
| | | cttttcagag cagcatagca tcaggattca |
| | | taggagtttg caaaagtatg ggctcaaaat |
| | | gtgtaagaga tgggaaagga ggatttaagt |
| | | atatcaggga tattacaagc aagattatct |
| | | tacaccgtga ttgtcacttt tgtaatcagc |
| | | gcaaaggtgt ttattgtaaa gcagcacttg |
| | | gtgaagtaag tgagtactcc agacccttaa |
| | | tttgggatta ttttgctctt gtgttaacca |
| | | atgcttgcga gttgggtaac tgggtgtttc |
| | | aaaaagctga agtacccaag atagtcactc |
| | | acctaaacaa tcccaatcac ttctggccaa |
| | | taaaaccaag cacacattct gagcttgagg |
| | | acaaggtggg catcaaccac atactttact |
| | | caatcaggag gaacttccct acactatttg |
| | | atgaacacat atcccctttc ctgtctgatt |
| | | tgaatatgct gaggctatca tgggttcaga |
| | | ggataaagtt cttagacctt tgtgtggcaa |
| | | ttgatataac atcagaatgt cttggtattg |
| | | ttagtcatat tataaaacat cgcagagaag |
| | | aattgtatat tgtaaagcaa aatgagcttg |
| | | caatgtccca tagcagagag tctcatccac |
| | | tggagagagg attcaacctt gaaccagaag |
| | | aagtgtgcac aaatttcttg atacagattt |
| | | tgtttgagtc aatgttggtg cctgtgatta |
| | | tgtcaacatc tcagttcaag aaatattttt |
| | | ggtttggtga gcttgaattg ttgccaaaca |
| | | atgcacaaca tgatctgaaa caattaaccc |
| | | agttcatttg tgactgcaaa aagaataaca |
| | | cttctaggac aatgaatctg gatgacttgg |
| | | atgttggctt tgtaagcagc aaactgattt |
| | | tgtcctgtgt taatttgaac attagtgtat |
| | | ttattaatga acttgactgg gtgaatagag |
| | | ataactatga gaacattgaa caactcattt |
| | | tagcatcacc ttctgaggtc attcccattg |
| | | agctcaacct aacattttct cataagagag |
| | | tgagccacaa atttagatat gagaggtcta |
| | | ccaattacat attaaaatta agatttctta |
| | | ttgaaaggga gtcattgctg gactcattgg |
| | | actctgatgg ttacctattg ttgaaccccc |
| | | attctgttga gtattatgtt tctcaatctt |
| | | ccgggaatca cataagtctg gatggtgtgt |
| | | cacttttggt tctgaatcct ctcatcaacg |
| | | gaaaagatgt tctagacttc aatgatctat |
| | | tggaaggtca agacattcac ttcaagtcaa |
| | | ggtcaactgt gttccagaag gtgagaatag |
| | | atttgaaaaa tcgtttcaag gatttgaaaa |
| | | acaaattttc ctataagttg attggtcctg |
| | | atgttgggat gcaacccctc attttagagg |
| | | gtgggttgat caaagagggc aacagagtgg |
| | | tttctagatt ggaagtgaac ttagactcaa |
| | | aggtggtcat cattgcactg gaagcattag |
| | | aacctgagaa aaggccaaga tttattgcca |
| | | acctttttca gtacctttca agtgcacagt |
| | | ctcacaacaa gggaataagt atgaacgagc |
| | | aggatttaag actcatgatt gaaaatttcc |
| | | ccgaagtgtt tgaacacatg ttgcatgatg |
| | | ccaaagattg gttgaattgt ggtcacttta |
| | | gtataataag aagtaagact ctaggatctg |
| | | ttatgattgc agacgagaca ggacccttca |
| | | agatcaaggg aatcaggtgt cgaaagttgt |
| | | ttgaggacaa tgagtcagtg gagatcgagt |
| | | aaacaaagag acgGCTAGC |
| 4 | PIC-L-GFP-Bsm: | gcgcaccgag gatcctaggc atttcttgat |
| | Representative cDNA | cagagacgat ggtgagcaag ggcgaggagc |
| | of modified L | tgttcaccgg ggtggtgccc atcctggtcg |
| | segment of Pichinde | agctggacgg cgacgtaaac ggccacaagt |
| | virus strain | tcagcgtgtc cggcgagggc gagggcgatg |
| | Munchique CoAn4763 | ccacctacgg caagctgacc ctgaagttca |
| | isolate P18 (Genbank | tctgcaccac cggcaagctg cccgtgccct |
| | accession number | ggcccaccct cgtgaccacc ttgacctacg |
| | EF529747.1), wherein | gcgtgcagtg cttcgtccgc taccccgacc |
| | the L ORF was | acatgaagca gcacgacttc ttcaagtccg |
| | deleted and | ccatgcccga aggctacgtc caggagcgca |
| | substituted by a GFP | ccatcttctt caaggacgac ggcaactaca |
| | ORF with flanking | agacccgcgc cgaggtgaag ttcgagggcg |
| | BsmBI sites (bold)on | acaccctggt gaaccgcatc gagctgaagg |
| | each side. | gcatcgactt caaggaggac ggcaacatcc |
| | | tggggcacaa gctggagtac aactacaaca |
| | | gccacaaggt ctatatcacc gccgacaagc |
| | | agaagaacgg catcaaggtg aacttcaaga |
| | | cccgccacaa catcgaggac ggcagcgtgc |
| | | agctcgccga ccactaccag cagaacaccc |
| | | ccatcggcga cggccccgtg ctgctgcccg |
| | | acaaccacta cctgagcacc cagtccgccc |
| | | tgagcaaaga ccccaacgag aagcgcgatc |
| | | acatggtcct gctggagttc gtgaccgccg |
| | | ccgggatcac tctcggcatg gacgagctgt |
| | | acaagtaacg tctctacaac cggccccatg |
| | | gggccggggg cccccgggcg cacccccgga |
| | | ggggggtgcg cccaggggcc ctggtttatg |
| | | gctcgtaggg tggtgcagag gggctttcta |
| | | ggaactccat cttggttggc agtgagtggc |
| | | cgcatatctc gcagagattg cctctggagt |
| | | gcattttggt taagcaccca agacacagat |
| | | agtggtcttt gcacctgatg agacctttgt |
| | | tgacgaacca gcaagatttg cagttgaacc |
| | | tgccatacag gccctgtggt agattgaggg |
| | | tcatggggac ccttcccacc acatcttcgt |
| | | cgccatgtct cttcctgacc tctttgctat |
| | | atctgagtcc catcccaagt tttgaccagg |
| | | acttgagctg ggttcaaatt ggacaaattt |
| | | gaagcgtgac ccaaagatgc ctaggatccc |
| | | cggtgcg |
| 5 | PIC-miniS-GFP: | gcgcaccggg gatcctaggc ataccttgga |
| | Representative cDNA | cgcgcatatt acttgatcaa agagagacga |
| | of a modified S | ggcctcgtct ctgccctagc ctcgacatgg |
| | segment cDNA of | gcctcgacgt cactccccaa taggggagtg |
| | Pichinde virus | acgtcgaggc ctctgaggac ttgagcatgt |
| | strain Munchique | cttcttactt gtacagctcg tccatgccga |
| | CoAn4763 isolate P18 | gagtgatccc ggcggcggtc acgaactcca |
| | (Genbank accession | gcaggaccat gtgatcgcgc ttctcgttgg |
| | number: EF529746.1), | ggtctttgct cagggcggac tgggtgctca |
| | wherein the GP ORF | ggtagtggtt gtcgggcagc agcacggggc |
| | was replaced by two | cgtcgccgat gggggtgttc tgctggtagt |
| | BsmBI restriction | ggtcggcgag ctgcacgctg ccgtcctcga |
| | sites (bold) and the | tgttgtggcg ggtcttgaag ttcaccttga |
| | NP ORF was replaced | tgccgttctt ctgcttgtcg gcggtgatat |
| | by GFP with two | agaccttgtg gctgttgtag ttgtactcca |
| | flanking BbsI | gcttgtgccc caggatgttg ccgtcctcct |
| | restriction sites | tgaagtcgat gcccttcagc tcgatgcggt |
| | (italicized). | tcaccagggt gtcgccctcg aacttcacct |
| | | cggcgcgggt cttgtagttg ccgtcgtcct |
| | | tgaagaagat ggtgcgctcc tggacgtagc |
| | | cttcgggcat ggcggacttg aagaagtcgt |
| | | gctgcttcat gtggtcgggg tagcggacga |
| | | agcactgcac gccgtaggtc aaggtggtca |
| | | cgagggtggg ccagggcacg ggcagcttgc |
| | | cggtggtgca gatgaacttc agggtcagct |
| | | tgccgtaggt ggcatcgccc tcgccctcgc |
| | | cggacacgct gaacttgtgg ccgtttacgt |
| | | cgccgtccag ctcgaccagg atgggcacca |
| | | ccccggtgaa cagctcctcg cccttgctca |
| | | ccatgaagac attttggggt tgtttgcact |
| | | tcctccgagt cagtgaagaa gtgaacgtac |
| | | agcgtgatct agaatcgcct aggatccact |
| | | gtgcg |
| 6 | NPΔBbsI: | GAATTCgaag acatcaaaat gtctgacaac |
| | Representative cDNA | atcccatcat tccgctgggt acagtccctt |
| | of a modified NP ORF | aggaggggtc tatccaactg gacccatcct |
| | of Pichinde virus | gtgaaggctg atgtgttgtc ggacacaaga |
| | strain Munchique | gcactgttat ctgctcttga ctttcacaaa |
| | CoAn4763 isolate P18 | gttgctcaag ttcaaagaat gatgcgcaaa |
| | (Genbank accession | gataaaagga ctgattctga tctgaccaag |
| | number EF529747.1), | ttaagagaca tgaacaaaga ggttgatgct |
| | wherein non-coding | ctgatgaata tgagatcaat ccagagggac |
| | mutation were | aatgtgctta aggtgggagg cttagccaaa |
| | introduced to delete | gaggagctaa tggagcttgc atctgatttg |
| | both BbsI | gacaagttaa gaaagaaagt cactagaact |
| | restriction sites | gagagtttgt ctcagcctgg tgtttatggg |
| | (italicized). This | ggcaatctca caaacactca gttggaacaa |
| | ORF also contains | agagccgaaa tccttcgctc aatggggttc |
| | flanking BbsI as | gctaatgcta gacccacagg caacagagat |
| | well as EcoRI | ggggttgtga agatctggga catcaaggat |
| | (uppercase)and NheI | aatacattgt tgatcaatca atttggatca |
| | (uppercase and | atgccagcct taaccatcgc ttgtatgact |
| | italicized) | gagcaagggg gtgaacaact taatgatgtt |
| | restriction sites | gtccaagcgc tgagtgcact tggtttgctc |
| | | tacactgtca agttcccgaa catgacagat |
| | | ctagagaaac tcacacagca acacagtgcc |
| | | ctaaaaatca ttagtaatga gccatcagcc |
| | | ataaacatct cagggtacaa tctcagtttg |
| | | tctgcagcag tcaaagcagc tgcttgcatg |
| | | attgatggtg gcaatatgct tgagaccatc |
| | | caggtgaagc cttctatgtt tagtactctc |
| | | ataaagagtc tattgcaaat aaagaatcgt |
| | | gaaggtatgt ttgtgagcac tacacccgga |
| | | cagagaaatc cttatgaaaa tttactatac |
| | | aagatttgtc tttcagggga tggttggcct |
| | | tacattggct caaggtctca agttcaaggg |
| | | agggcttggg ataacaccac tgtagattta |
| | | gattcgaagc cgagtgctat ccagccacca |
| | | gtaagaaacg gaggatcacc ggaccttaaa |
| | | caaatcccta aggagaaaga agatactgtt |
| | | gtgtcctcaa ttcagatgct tgattcaaaa |
| | | gctaccacat ggattgacat tgaaggaaca |
| | | ccaaatgatc cggtggaaat ggccatctac |
| | | cagcctgaca cgggcaacta catacattgt |
| | | tacagatttc cccacgatga gaagtccttc |
| | | aaagagcaaa gcaagtactc acatggtctc |
| | | cttttaaagg acttggctga tgcccaacca |
| | | ggcctgattt cctcaatcat cagacattta |
| | | cctcaaaaca tggttttcac tgctcaaggt |
| | | tcagatgata taatcagttt gttcgaaatg |
| | | catgggagaa gagacttaaa agtgcttgac |
| | | gtgaaactca gtgccgagca agcacgcacc |
| | | tttgaggatg agatctggga gagatacaat |
| | | ctactctgca ccaaacataa aggtttggtc |
| | | ataaagaaga agaagaaggg ggctgcacaa |
| | | accactgcga atcctcactg tgcattgctt |
| | | gataccatca tgtttgatgc aacagtgaca |
| | | ggctgggtta gggaccagaa gccgatgaga |
| | | tgcttgccta ttgacacgct gtacaggaac |
| | | aacacagatc tgatcaacct ctgagctcat |
| | | gtcttcGCTA GC |
| 7 | PIC-NP-Bsm: | gcgcaccggg gatcctaggc ataccttgga |
| | Representative cDNA | cgcgcatatt acttgatcaa agagagacga |
| | obtained when | ggcctcgtct ctgccctagc ctcgacatgg |
| | NPΔBbsI was digested | gcctcgacgt cactccccaa taggggagtg |
| | with BbsI to insert | acgtcgaggc ctctgaggac ttgagctcag |
| | the BbsI-mutated NP | aggttgatca gatctgtgtt gttcctgtac |
| | ORF into the equally | agcgtgtcaa taggcaagca tctcatcggc |
| | digested pol-I-PIC- | ttctggtccc taacccagcc tgtcactgtt |
| | miniS-GFP backbone, | gcatcaaaca tgatggtatc aagcaatgca |
| | thereby replacing | cagtgaggat tcgcagtggt ttgtgcagcc |
| | the GFP ORF with the | cccttcttct tcttctttat gaccaaacct |
| | NP ORF. | ttatgtttgg tgcagagtag attgtatctc |
| | | tcccagatct catcctcaaa ggtgcgtgct |
| | | tgctcggcac tgagtttcac gtcaagcact |
| | | tttaagtctc ttctcccatg catttcgaac |
| | | aaactgatta tatcatctga accttgagca |
| | | gtgaaaacca tgttttgagg taaatgtctg |
| | | atgattgagg aaatcaggcc tggttgggca |
| | | tcagccaagt cctttaaaag gagaccatgt |
| | | gagtacttgc tttgctcttt gaaggacttc |
| | | tcatcgtggg gaaatctgta acaatgtatg |
| | | tagttgcccg tgtcaggctg gtagatggcc |
| | | atttccaccg gatcatttgg tgttccttca |
| | | atgtcaatcc atgtggtagc ttttgaatca |
| | | agcatctgaa ttgaggacac aacagtatct |
| | | tctttctcct tagggatttg tttaaggtcc |
| | | ggtgatcctc cgtttcttac tggtggctgg |
| | | atagcactcg gcttcgaatc taaatctaca |
| | | gtggtgttat cccaagccct cccttgaact |
| | | tgagaccttg agccaatgta aggccaacca |
| | | tcccctgaaa gacaaatctt gtatagtaaa |
| | | ttttcataag gatttctctg tccgggtgta |
| | | gtgctcacaa acataccttc acgattcttt |
| | | atttgcaata gactctttat gagagtacta |
| | | aacatagaag gcttcacctg gatggtctca |
| | | agcatattgc caccatcaat catgcaagca |
| | | gctgctttga ctgctgcaga caaactgaga |
| | | ttgtaccctg agatgtttat ggctgatggc |
| | | tcattactaa tgatttttag ggcactgtgt |
| | | tgctgtgtga gtttctctag atctgtcatg |
| | | ttcgggaact tgacagtgta gagcaaacca |
| | | agtgcactca gcgcttggac aacatcatta |
| | | agttgttcac ccccttgctc agtcatacaa |
| | | gcgatggtta aggctggcat tgatccaaat |
| | | tgattgatca acaatgtatt atccttgatg |
| | | tcccagatct tcacaacccc atctctgttg |
| | | cctgtgggtc tagcattagc gaaccccatt |
| | | gagcgaagga tttcggctct ttgttccaac |
| | | tgagtgtttg tgagattgcc cccataaaca |
| | | ccaggctgag acaaactctc agttctagtg |
| | | actttctttc ttaacttgtc caaatcagat |
| | | gcaagctcca ttagctcctc tttggctaag |
| | | cctcccacct taagcacatt gtccctctgg |
| | | attgatctca tattcatcag agcatcaacc |
| | | tctttgttca tgtctcttaa cttggtcaga |
| | | tcagaatcag tccttttatc tttgcgcatc |
| | | attctttgaa cttgagcaac tttgtgaaag |
| | | tcaagagcag ataacagtgc tcttgtgtcc |
| | | gacaacacat cagccttcac aggatgggtc |
| | | cagttggata gacccctcct aagggactgt |
| | | acccagcgga atgatgggat gttgtcagac |
| | | attttggggt tgtttgcact tcctccgagt |
| | | cagtgaagaa gtgaacgtac agcgtgatct |
| | | agaatcgcct aggatccact gtgcg |
| 8 | PIC-GP-Bsm: | gcgcaccggg gatcctaggc ataccttgga |
| | Representative cDNA | cgcgcatatt acttgatcaa agagagacga |
| | obtained when | ggcctcgtct ctgccctagc ctcgacatgg |
| | GPΔBbsI was digested | gcctcgacgt cactccccaa taggggagtg |
| | with BbsI to insert | acgtcgaggc ctctgaggac ttgagcttat |
| | the BbsI-mutated GP | ttacccagtc tcacccattt gtagggtttc |
| | ORF into the equally | tttgggattt tataataccc acagctgcaa |
| | digested pol-I-PIC- | agagagttcc tagtaatcct atgtggcttc |
| | miniS-GFP backbone, | ggacagccat caccaatgat gtgcctatga |
| | thereby replacing | gtgggtattc caactaagtg gagaaacact |
| | the GFP ORF with the | gtgatggtgt aaaacaccaa agaccagaag |
| | GP ORF. | caaatgtctg tcaatgctag tggagtctta |
| | | ccttgtcttt cttcatattc ttttatcagc |
| | | atttcattgt acagattctg gctctcccac |
| | | aaccaatcat tcttaaaatg cgtttcattg |
| | | aggtacgagc cattgtgaac taaccaacac |
| | | tgcggtaaag aatgtctccc tgtgatggta |
| | | tcattgatgt accaaaattt tgtatagttg |
| | | caataaggga ttttggcaag ctgtttgaga |
| | | ctgtttctaa tcacaagtga gtcagaaata |
| | | agtccgttga tagtcttttt aaagagattc |
| | | aacgaattct caacattaag ttgtaaggtt |
| | | ttgatagcat tctgattgaa atcaaataac |
| | | ctcatcgtat cgcaaaattc ttcattgtga |
| | | tctttgttgc attttgccat cacagtgtta |
| | | tcaaaacatt ttattccagc ccaaacaata |
| | | gcccattgct ccaaacagta accacctggg |
| | | acatgttgcc cagtagagtc actcaagtcc |
| | | caagtgaaaa agccaaggag tttcctgctc |
| | | acagaactat aagcagtttt ttggagagcc |
| | | atccttattg ttgccattgg agtatatgta |
| | | cagtgatttt cccatgtggt gttctgtatg |
| | | atcaggaaat tgtaatgtgt cccaccttca |
| | | cagtttgtta gtctgcaaga ccctccacta |
| | | cagttattga aacattttcc aacccacgca |
| | | atttttgggt ccccaatgat ttgagcaagc |
| | | gacgcaataa gatgtctgcc aacctcacct |
| | | cctctatccc caactgtcaa gttgtactgg |
| | | atcaacaccc cagcaccctc aactgttttg |
| | | catctggcac ctacatgacg agtgacatgg |
| | | agcacattga agtgtaactc attaagcaac |
| | | cattttaatg tgtgacctgc ttcttctgtc |
| | | ttatcacaat tactaatgtt accatatgca |
| | | aggcttctga tgttggaaaa gtttccagta |
| | | gtttcatttg caatggatgt gtttgtcaaa |
| | | gtgagttcaa ttccccatgt tgtgttagat |
| | | ggtcctttgt agtaatgatg tgtgttgttc |
| | | ttgctacatg attgtggcaa gttgtcaaac |
| | | attcttgtga ggttgaactc aacgtgggtg |
| | | agattgtgcc tcctatcaat catcatgcca |
| | | tcacaacttc tgccagccaa aatgaggaag |
| | | gtgatgagtt ggaataggcc acatctcatc |
| | | agattgacaa atcctttgat gatgcatagg |
| | | gttgagacaa tgattaaggc gacattgaac |
| | | acctcctgca ggacttcggg tatagactgg |
| | | atcaaagtca caacttgtcc cattttgggg |
| | | ttgtttgcac ttcctccgag tcagtgaaga |
| | | agtgaacgta cagcgtgatc tagaatcgcc |
| | | taggatccac tgtgcg |
| 9 | GFP-Bsm: Green | cgtctctaaa gatggtgagc aagggcgagg |
| | fluorescent | agctgttcac cggggtggtg cccatcctgg |
| | protein (GFP) | tcgagctgga cggcgacgta aacggccaca |
| | synthesized with | agttcagcgt gtccggcgag ggcgagggcg |
| | flanking BsmBI sites | atgccaccta cggcaagctg accctgaagt |
| | (bold). | tcatctgcac caccggcaag ctgcccgtgc |
| | | cctggcccac cctcgtgacc accttgacct |
| | | acggcgtgca gtgcttcgtc cgctaccccg |
| | | accacatgaa gcagcacgac ttcttcaagt |
| | | ccgccatgcc cgaaggctac gtccaggagc |
| | | gcaccatctt cttcaaggac gacggcaact |
| | | acaagacccg cgccgaggtg aagttcgagg |
| | | gcgacaccct ggtgaaccgc atcgagctga |
| | | agggcatcga cttcaaggag gacggcaaca |
| | | tcctggggca caagctggag tacaactaca |
| | | acagccacaa ggtctatatc accgccgaca |
| | | agcagaagaa cggcatcaag gtgaacttca |
| | | agacccgcca caacatcgag gacggcagcg |
| | | tgcagctcgc cgaccactac cagcagaaca |
| | | cccccatcgg cgacggcccc gtgctgctgc |
| | | ccgacaacca ctacctgagc acccagtccg |
| | | ccctgagcaa agaccccaac gagaagcgcg |
| | | atcacatggt cctgctggag ttcgtgaccg |
| | | ccgccgggat cactctcggc atggacgagc |
| | | tgtacaagta agcccagaga cg |
| 10 | sP1AGM-Bsm: Fusion | cgtctctaag gatgaaatgc ctcctctacc |
| | protein consisting | ttgcatttct cttcattgga gtcaactgca |
| | of i) the vesicular | tgagtgacaa caagaagcct gacaaggccc |
| | stomatitis virus | actctggcag tggaggagat ggtgatggca |
| | glycoprotein (VSVG) | acagatgcaa cctgctgcac agatacagcc |
| | signal peptide, ii) | tggaagagat cctgccctac ctgggctggc |
| | the P1A antigen of | tggtgtttgc tgtggtgaca acaagcttcc |
| | the P815 mouse | tggccctgca gatgttcatt gatgccctgt |
| | mastocytoma tumor | atgaggaaca gtatgagagg gatgtggcct |
| | cell line, iii) a | ggattgccag acagagcaag agaatgagca |
| | GSG linker, iv) an | gtgtggatga ggatgaggat gatgaggatg |
| | enterovirus 2A | atgaagatga ctactatgat gatgaggatg |
| | peptide, and v) | atgatgatga tgccttctat gatgatgagg |
| | mouse GM-CSF | atgatgaaga ggaagaactg gaaaacctga |
| | synthesized with | tggatgatga gtctgaggat gaggctgagg |
| | flanking BsmBI sites | aagagatgag tgtggaaatg ggggctgggg |
| | (bold). | cagaagagat gggagcaggt gccaactgtg |
| | | cttgtgtgcc aggacaccac ctgagaaaga |
| | | atgaagtgaa gtgcaggatg atctacttct |
| | | tccatgaccc caactttctg gtgtccatcc |
| | | ctgtgaaccc caaagaacag atggaatgca |
| | | gatgtgagaa tgcagatgaa gaggtggcca |
| | | tggaagaaga agaggaagag gaagaagaag |
| | | aagaagagga agaaatgggc aacccagatg |
| | | gcttcagccc tggaagtggt caccatcacc |
| | | accatcatgg cagtggggca accaacttca |
| | | gcctgctgaa acaggctggg gatgtggaag |
| | | aaaatcctgg ccccatgtgg ctccagaatc |
| | | tgctttttct gggcattgtg gtttacagcc |
| | | tgagtgcacc cacaagatct cccatcacag |
| | | tgacaagacc ttggaagcat gtggaagcaa |
| | | tcaaagaggc cctgaatctg cttgatgaca |
| | | tgccagtgac cctgaatgaa gaagtggaag |
| | | tggtgtcaaa tgagttcagc ttcaaaaaac |
| | | tgacctgtgt gcagaccagg ctgaaaattt |
| | | ttgaacaggg cctgagagga aacttcacaa |
| | | agctgaaggg agctctgaac atgactgcca |
| | | gctactacca gacctactgc ccccccaccc |
| | | cagagacaga ttgtgagaca caagtgacca |
| | | cctatgctga cttcattgac agcctgaaaa |
| | | ccttcctgac tgacatcccc tttgagtgca |
| | | agaaacctgt gcagaagtga agaaagagac |
| | | g |
| 11 | PIC-NP-GFP (S- | gcgcaccggg gatcctaggc ataccttgga |
| | NP/GFP) | cgcgcatatt acttgatcaa agatggtgag |
| | | caagggcgag gagctgttca ccggggtggt |
| | | gcccatcctg gtcgagctgg acggcgacgt |
| | | aaacggccac aagttcagcg tgtccggcga |
| | | gggcgagggc gatgccacct acggcaagct |
| | | gaccctgaag ttcatctgca ccaccggcaa |
| | | gctgcccgtg ccctggccca ccctcgtgac |
| | | caccttgacc tacggcgtgc agtgcttcgt |
| | | ccgctacccc gaccacatga agcagcacga |
| | | cttcttcaag tccgccatgc ccgaaggcta |
| | | cgtccaggag cgcaccatct tcttcaagga |
| | | cgacggcaac tacaagaccc gcgccgaggt |
| | | gaagttcgag ggcgacaccc tggtgaaccg |
| | | catcgagctg aagggcatcg acttcaagga |
| | | ggacggcaac atcctggggc acaagctgga |
| | | gtacaactac aacagccaca aggtctatat |
| | | caccgccgac aagcagaaga acggcatcaa |
| | | ggtgaacttc aagacccgcc acaacatcga |
| | | ggacggcagc gtgcagctcg ccgaccacta |
| | | ccagcagaac acccccatcg gcgacggccc |
| | | cgtgctgctg cccgacaacc actacctgag |
| | | cacccagtcc gccctgagca aagaccccaa |
| | | cgagaagcgc gatcacatgg tcctgctgga |
| | | gttcgtgacc gccgccggga tcactctcgg |
| | | catggacgag ctgtacaagt aagccctagc |
| | | ctcgacatgg gcctcgacgt cactccccaa |
| | | taggggagtg acgtcgaggc ctctgaggac |
| | | ttgagctcag aggttgatca gatctgtgtt |
| | | gttcctgtac agcgtgtcaa taggcaagca |
| | | tctcatcggc ttctggtccc taacccagcc |
| | | tgtcactgtt gcatcaaaca tgatggtatc |
| | | aagcaatgca cagtgaggat tcgcagtggt |
| | | ttgtgcagcc cccttcttct tcttctttat |
| | | gaccaaacct ttatgtttgg tgcagagtag |
| | | attgtatctc tcccagatct catcctcaaa |
| | | ggtgcgtgct tgctcggcac tgagtttcac |
| | | gtcaagcact tttaagtctc ttctcccatg |
| | | catttcgaac aaactgatta tatcatctga |
| | | accttgagca gtgaaaacca tgttttgagg |
| | | taaatgtctg atgattgagg aaatcaggcc |
| | | tggttgggca tcagccaagt cctttaaaag |
| | | gagaccatgt gagtacttgc tttgctcttt |
| | | gaaggacttc tcatcgtggg gaaatctgta |
| | | acaatgtatg tagttgcccg tgtcaggctg |
| | | gtagatggcc atttccaccg gatcatttgg |
| | | tgttccttca atgtcaatcc atgtggtagc |
| | | ttttgaatca agcatctgaa ttgaggacac |
| | | aacagtatct tctttctcct tagggatttg |
| | | tttaaggtcc ggtgatcctc cgtttcttac |
| | | tggtggctgg atagcactcg gcttcgaatc |
| | | taaatctaca gtggtgttat cccaagccct |
| | | cccttgaact tgagaccttg agccaatgta |
| | | aggccaacca tcccctgaaa gacaaatctt |
| | | gtatagtaaa ttttcataag gatttctctg |
| | | tccgggtgta gtgctcacaa acataccttc |
| | | acgattcttt atttgcaata gactctttat |
| | | gagagtacta aacatagaag gcttcacctg |
| | | gatggtctca agcatattgc caccatcaat |
| | | catgcaagca gctgctttga ctgctgcaga |
| | | caaactgaga ttgtaccctg agatgtttat |
| | | ggctgatggc tcattactaa tgatttttag |
| | | ggcactgtgt tgctgtgtga gtttctctag |
| | | atctgtcatg ttcgggaact tgacagtgta |
| | | gagcaaacca agtgcactca gcgcttggac |
| | | aacatcatta agttgttcac ccccttgctc |
| | | agtcatacaa gcgatggtta aggctggcat |
| | | tgatccaaat tgattgatca acaatgtatt |
| | | atccttgatg tcccagatct tcacaacccc |
| | | atctctgttg cctgtgggtc tagcattagc |
| | | gaaccccatt gagcgaagga tttcggctct |
| | | ttgttccaac tgagtgtttg tgagattgcc |
| | | cccataaaca ccaggctgag acaaactctc |
| | | agttctagtg actttctttc ttaacttgtc |
| | | caaatcagat gcaagctcca ttagctcctc |
| | | tttggctaag cctcccacct taagcacatt |
| | | gtccctctgg attgatctca tattcatcag |
| | | agcatcaacc tctttgttca tgtctcttaa |
| | | cttggtcaga tcagaatcag tccttttatc |
| | | tttgcgcatc attctttgaa cttgagcaac |
| | | tttgtgaaag tcaagagcag ataacagtgc |
| | | tcttgtgtcc gacaacacat cagccttcac |
| | | aggatgggtc cagttggata gacccctcct |
| | | aagggactgt acccagcgga atgatgggat |
| | | gttgtcagac attttggggt tgtttgcact |
| | | tcctccgagt cagtgaagaa gtgaacgtac |
| | | agcgtgatct agaatcgcct aggatccact gtgcg |
| 12 | PIC-NP-sP1AGM | gcgcaccggg gatcctaggc ataccttgga |
| | | cgcgcatatt acttgatcaa agatgaaatg |
| | | cctcctctac cttgcatttc tcttcattgg |
| | | agtcaactgc atgagtgaca acaagaagcc |
| | | tgacaaggcc cactctggca gtggaggaga |
| | | tggtgatggc aacagatgca acctgctgca |
| | | cagatacagc ctggaagaga tcctgcccta |
| | | cctgggctgg ctggtgtttg ctgtggtgac |
| | | aacaagcttc ctggccctgc agatgttcat |
| | | tgatgccctg tatgaggaac agtatgagag |
| | | ggatgtggcc tggattgcca gacagagcaa |
| | | gagaatgagc agtgtggatg aggatgagga |
| | | tgatgaggat gatgaagatg actactatga |
| | | tgatgaggat gatgatgatg atgccttcta |
| | | tgatgatgag gatgatgaag aggaagaact |
| | | ggaaaacctg atggatgatg agtctgagga |
| | | tgaggctgag gaagagatga gtgtggaaat |
| | | gggggctggg gcagaagaga tgggagcagg |
| | | tgccaactgt gcttgtgtgc caggacacca |
| | | cctgagaaag aatgaagtga agtgcaggat |
| | | gatctacttc ttccatgacc ccaactttct |
| | | ggtgtccatc cctgtgaacc ccaaagaaca |
| | | gatggaatgc agatgtgaga atgcagatga |
| | | agaggtggcc atggaagaag aagaggaaga |
| | | ggaagaagaa gaagaagagg aagaaatggg |
| | | caacccagat ggcttcagcc ctggaagtgg |
| | | tcaccatcac caccatcatg gcagtggggc |
| | | aaccaacttc agcctgctga aacaggctgg |
| | | ggatgtggaa gaaaatcctg gcccctggct |
| | | ccagaatctg ctttttctgg gcattgtggt |
| | | ttacagcctg agtgcaccca caagatctcc |
| | | catcacagtg acaagacctt ggaagcatgt |
| | | ggaagcaatc aaagaggccc tgaatctgct |
| | | tgatgacatg ccagtgaccc tgaatgaaga |
| | | agtggaagtg gtgtcaaatg agttcagctt |
| | | caaaaaactg acctgtgtgc agaccaggct |
| | | gaaaattttt gaacagggcc tgagaggaaa |
| | | cttcacaaag ctgaagggag ctctgaacat |
| | | gactgccagc tactaccaga cctactgccc |
| | | ccccacccca gagacagatt gtgagacaca |
| | | agtgaccacc tatgctgact tcattgacag |
| | | cctgaaaacc ttcctgactg acatcccctt |
| | | tgagtgcaag aaacctgtgc agaagtgagc |
| | | cctagcctcg acatgggcct cgacgtcact |
| | | ccccaatagg ggagtgacgt cgaggcctct |
| | | gaggacttga gctcagaggt tgatcagatc |
| | | tgtgttgttc ctgtacagcg tgtcaatagg |
| | | caagcatctc atcggcttct ggtccctaac |
| | | ccagcctgtc actgttgcat caaacatgat |
| | | ggtatcaagc aatgcacagt gaggattcgc |
| | | agtggtttgt gcagccccct tcttcttctt |
| | | ctttatgacc aaacctttat gtttggtgca |
| | | gagtagattg tatctctccc agatctcatc |
| | | ctcaaaggtg cgtgcttgct cggcactgag |
| | | tttcacgtca agcactttta agtctcttct |
| | | cccatgcatt tcgaacaaac tgattatatc |
| | | atctgaacct tgagcagtga aaaccatgtt |
| | | ttgaggtaaa tgtctgatga ttgaggaaat |
| | | caggcctggt tgggcatcag ccaagtcctt |
| | | taaaaggaga ccatgtgagt acttgctttg |
| | | ctctttgaag gacttctcat cgtggggaaa |
| | | tctgtaacaa tgtatgtagt tgcccgtgtc |
| | | aggctggtag atggccattt ccaccggatc |
| | | atttggtgtt ccttcaatgt caatccatgt |
| | | ggtagctttt gaatcaagca tctgaattga |
| | | ggacacaaca gtatcttctt tctccttagg |
| | | gatttgttta aggtccggtg atcctccgtt |
| | | tcttactggt ggctggatag cactcggctt |
| | | cgaatctaaa tctacagtgg tgttatccca |
| | | agccctccct tgaacttgag accttgagcc |
| | | aatgtaaggc caaccatccc ctgaaagaca |
| | | aatcttgtat agtaaatttt cataaggatt |
| | | tctctgtccg ggtgtagtgc tcacaaacat |
| | | accttcacga ttctttattt gcaatagact |
| | | ctttatgaga gtactaaaca tagaaggctt |
| | | cacctggatg gtctcaagca tattgccacc |
| | | atcaatcatg caagcagctg ctttgactgc |
| | | tgcagacaaa ctgagattgt accctgagat |
| | | gtttatggct gatggctcat tactaatgat |
| | | ttttagggca ctgtgttgct gtgtgagttt |
| | | ctctagatct gtcatgttcg ggaacttgac |
| | | agtgtagagc aaaccaagtg cactcagcgc |
| | | ttggacaaca tcattaagtt gttcaccccc |
| | | ttgctcagtc atacaagcga tggttaaggc |
| | | tggcattgat ccaaattgat tgatcaacaa |
| | | tgtattatcc ttgatgtccc agatcttcac |
| | | aaccccatct ctgttgcctg tgggtctagc |
| | | attagcgaac cccattgagc gaaggatttc |
| | | ggctctttgt tccaactgag tgtttgtgag |
| | | attgccccca taaacaccag gctgagacaa |
| | | actctcagtt ctagtgactt tctttcttaa |
| | | cttgtccaaa tcagatgcaa gctccattag |
| | | ctcctctttg gctaagcctc ccaccttaag |
| | | cacattgtcc ctctggattg atctcatatt |
| | | catcagagca tcaacctctt tgttcatgtc |
| | | tcttaacttg gtcagatcag aatcagtcct |
| | | tttatctttg cgcatcattc tttgaacttg |
| | | agcaactttg tgaaagtcaa gagcagataa |
| | | cagtgctctt gtgtccgaca acacatcagc |
| | | cttcacagga tgggtccagt tggatagacc |
| | | cctcctaagg gactgtaccc agcggaatga |
| | | tgggatgttg tcagacattt tggggttgtt |
| | | tgcacttcct ccgagtcagt gaagaagtga |
| | | acgtacagcg tgatctagaa tcgcctagga |
| | | tccactgtgc g |
| 13 | PIC-GP-GFP (S- | gcgcaccggg gatcctaggc ataccttgga |
| | GP/GFPart) | cgcgcatatt acttgatcaa agatggtgag |
| | | caagggcgag gagctgttca ccggggtggt |
| | | gcccatcctg gtcgagctgg acggcgacgt |
| | | aaacggccac aagttcagcg tgtccggcga |
| | | gggcgagggc gatgccacct acggcaagct |
| | | gaccctgaag ttcatctgca ccaccggcaa |
| | | gctgcccgtg ccctggccca ccctcgtgac |
| | | caccttgacc tacggcgtgc agtgcttcgt |
| | | ccgctacccc gaccacatga agcagcacga |
| | | cttcttcaag tccgccatgc ccgaaggcta |
| | | cgtccaggag cgcaccatct tcttcaagga |
| | | cgacggcaac tacaagaccc gcgccgaggt |
| | | gaagttcgag ggcgacaccc tggtgaaccg |
| | | catcgagctg aagggcatcg acttcaagga |
| | | ggacggcaac atcctggggc acaagctgga |
| | | gtacaactac aacagccaca aggtctatat |
| | | caccgccgac aagcagaaga acggcatcaa |
| | | ggtgaacttc aagacccgcc acaacatcga |
| | | ggacggcagc gtgcagctcg ccgaccacta |
| | | ccagcagaac acccccatcg gcgacggccc |
| | | cgtgctgctg cccgacaacc actacctgag |
| | | cacccagtcc gccctgagca aagaccccaa |
| | | cgagaagcgc gatcacatgg tcctgctgga |
| | | gttcgtgacc gccgccggga tcactctcgg |
| | | catggacgag ctgtacaagt aagccctagc |
| | | ctcgacatgg gcctcgacgt cactccccaa |
| | | taggggagtg acgtcgaggc ctctgaggac |
| | | ttgagcttat ttacccagtc tcacccattt |
| | | gtagggtttc tttgggattt tataataccc |
| | | acagctgcaa agagagttcc tagtaatcct |
| | | atgtggcttc ggacagccat caccaatgat |
| | | gtgcctatga gtgggtattc caactaagtg |
| | | gagaaacact gtgatggtgt aaaacaccaa |
| | | agaccagaag caaatgtctg tcaatgctag |
| | | tggagtctta ccttgtcttt cttcatattc |
| | | ttttatcagc atttcattgt acagattctg |
| | | gctctcccac aaccaatcat tcttaaaatg |
| | | cgtttcattg aggtacgagc cattgtgaac |
| | | taaccaacac tgcggtaaag aatgtctccc |
| | | tgtgatggta tcattgatgt accaaaattt |
| | | tgtatagttg caataaggga ttttggcaag |
| | | ctgtttgaga ctgtttctaa tcacaagtga |
| | | gtcagaaata agtccgttga tagtcttttt |
| | | aaagagattc aacgaattct caacattaag |
| | | ttgtaaggtt ttgatagcat tctgattgaa |
| | | atcaaataac ctcatcgtat cgcaaaattc |
| | | ttcattgtga tctttgttgc attttgccat |
| | | cacagtgtta tcaaaacatt ttattccagc |
| | | ccaaacaata gcccattgct ccaaacagta |
| | | accacctggg acatgttgcc cagtagagtc |
| | | actcaagtcc caagtgaaaa agccaaggag |
| | | tttcctgctc acagaactat aagcagtttt |
| | | ttggagagcc atccttattg ttgccattgg |
| | | agtatatgta cagtgatttt cccatgtggt |
| | | gttctgtatg atcaggaaat tgtaatgtgt |
| | | cccaccttca cagtttgtta gtctgcaaga |
| | | ccctccacta cagttattga aacattttcc |
| | | aacccacgca atttttgggt ccccaatgat |
| | | ttgagcaagc gacgcaataa gatgtctgcc |
| | | aacctcacct cctctatccc caactgtcaa |
| | | gttgtactgg atcaacaccc cagcaccctc |
| | | aactgttttg catctggcac ctacatgacg |
| | | agtgacatgg agcacattga agtgtaactc |
| | | attaagcaac cattttaatg tgtgacctgc |
| | | ttcttctgtc ttatcacaat tactaatgtt |
| | | accatatgca aggcttctga tgttggaaaa |
| | | gtttccagta gtttcatttg caatggatgt |
| | | gtttgtcaaa gtgagttcaa ttccccatgt |
| | | tgtgttagat ggtcctttgt agtaatgatg |
| | | tgtgttgttc ttgctacatg attgtggcaa |
| | | gttgtcaaac attcttgtga ggttgaactc |
| | | aacgtgggtg agattgtgcc tcctatcaat |
| | | catcatgcca tcacaacttc tgccagccaa |
| | | aatgaggaag gtgatgagtt ggaataggcc |
| | | acatctcatc agattgacaa atcctttgat |
| | | gatgcatagg gttgagacaa tgattaaggc |
| | | gacattgaac acctcctgca ggacttcggg |
| | | tatagactgg atcaaagtca caacttgtcc |
| | | cattttgggg ttgtttgcac ttcctccgag |
| | | tcagtgaaga agtgaacgta cagcgtgatc |
| | | tagaatcgcc taggatccac tgtgcg |
| 14 | PIC-GP-sP1AGM | gcgcaccggg gatcctaggc ataccttgga |
| | | cgcgcatatt acttgatcaa agatgaaatg |
| | | cctcctctac cttgcatttc tcttcattgg |
| | | agtcaactgc atgagtgaca acaagaagcc |
| | | tgacaaggcc cactctggca gtggaggaga |
| | | tggtgatggc aacagatgca acctgctgca |
| | | cagatacagc ctggaagaga tcctgcccta |
| | | cctgggctgg ctggtgtttg ctgtggtgac |
| | | aacaagcttc ctggccctgc agatgttcat |
| | | tgatgccctg tatgaggaac agtatgagag |
| | | ggatgtggcc tggattgcca gacagagcaa |
| | | gagaatgagc agtgtggatg aggatgagga |
| | | tgatgaggat gatgaagatg actactatga |
| | | tgatgaggat gatgatgatg atgccttcta |
| | | tgatgatgag gatgatgaag aggaagaact |
| | | ggaaaacctg atggatgatg agtctgagga |
| | | tgaggctgag gaagagatga gtgtggaaat |
| | | gggggctggg gcagaagaga tgggagcagg |
| | | tgccaactgt gcttgtgtgc caggacacca |
| | | cctgagaaag aatgaagtga agtgcaggat |
| | | gatctacttc ttccatgacc ccaactttct |
| | | ggtgtccatc cctgtgaacc ccaaagaaca |
| | | gatggaatgc agatgtgaga atgcagatga |
| | | agaggtggcc atggaagaag aagaggaaga |
| | | ggaagaagaa gaagaagagg aagaaatggg |
| | | caacccagat ggcttcagcc ctggaagtgg |
| | | tcaccatcac caccatcatg gcagtggggc |
| | | aaccaacttc agcctgctga aacaggctgg |
| | | ggatgtggaa gaaaatcctg gcccctggct |
| | | ccagaatctg ctttttctgg gcattgtggt |
| | | ttacagcctg agtgcaccca caagatctcc |
| | | catcacagtg acaagacctt ggaagcatgt |
| | | ggaagcaatc aaagaggccc tgaatctgct |
| | | tgatgacatg ccagtgaccc tgaatgaaga |
| | | agtggaagtg gtgtcaaatg agttcagctt |
| | | caaaaaactg acctgtgtgc agaccaggct |
| | | gaaaattttt gaacagggcc tgagaggaaa |
| | | cttcacaaag ctgaagggag ctctgaacat |
| | | gactgccagc tactaccaga cctactgccc |
| | | ccccacccca gagacagatt gtgagacaca |
| | | agtgaccacc tatgctgact tcattgacag |
| | | cctgaaaacc ttcctgactg acatcccctt |
| | | tgagtgcaag aaacctgtgc agaagtgagc |
| | | cctagcctcg acatgggcct cgacgtcact |
| | | ccccaatagg ggagtgacgt cgaggcctct |
| | | gaggacttga gcttatttac ccagtctcac |
| | | ccatttgtag ggtttctttg ggattttata |
| | | atacccacag ctgcaaagag agttcctagt |
| | | aatcctatgt ggcttcggac agccatcacc |
| | | aatgatgtgc ctatgagtgg gtattccaac |
| | | taagtggaga aacactgtga tggtgtaaaa |
| | | caccaaagac cagaagcaaa tgtctgtcaa |
| | | tgctagtgga gtcttacctt gtctttcttc |
| | | atattctttt atcagcattt cattgtacag |
| | | attctggctc tcccacaacc aatcattctt |
| | | aaaatgcgtt tcattgaggt acgagccatt |
| | | gtgaactaac caacactgcg gtaaagaatg |
| | | tctccctgtg atggtatcat tgatgtacca |
| | | aaattttgta tagttgcaat aagggatttt |
| | | ggcaagctgt ttgagactgt ttctaatcac |
| | | aagtgagtca gaaataagtc cgttgatagt |
| | | ctttttaaag agattcaacg aattctcaac |
| | | attaagttgt aaggttttga tagcattctg |
| | | attgaaatca aataacctca tcgtatcgca |
| | | aaattcttca ttgtgatctt tgttgcattt |
| | | tgccatcaca gtgttatcaa aacattttat |
| | | tccagcccaa acaatagccc attgctccaa |
| | | acagtaacca cctgggacat gttgcccagt |
| | | agagtcactc aagtcccaag tgaaaaagcc |
| | | aaggagtttc ctgctcacag aactataagc |
| | | agttttttgg agagccatcc ttattgttgc |
| | | cattggagta tatgtacagt gattttccca |
| | | tgtggtgttc tgtatgatca ggaaattgta |
| | | atgtgtccca ccttcacagt ttgttagtct |
| | | gcaagaccct ccactacagt tattgaaaca |
| | | ttttccaacc cacgcaattt ttgggtcccc |
| | | aatgatttga gcaagcgacg caataagatg |
| | | tctgccaacc tcacctcctc tatccccaac |
| | | tgtcaagttg tactggatca acaccccagc |
| | | accctcaact gttttgcatc tggcacctac |
| | | atgacgagtg acatggagca cattgaagtg |
| | | taactcatta agcaaccatt ttaatgtgtg |
| | | acctgcttct tctgtcttat cacaattact |
| | | aatgttacca tatgcaaggc ttctgatgtt |
| | | ggaaaagttt ccagtagttt catttgcaat |
| | | ggatgtgttt gtcaaagtga gttcaattcc |
| | | ccatgttgtg ttagatggtc ctttgtagta |
| | | atgatgtgtg ttgttcttgc tacatgattg |
| | | tggcaagttg tcaaacattc ttgtgaggtt |
| | | gaactcaacg tgggtgagat tgtgcctcct |
| | | atcaatcatc atgccatcac aacttctgcc |
| | | agccaaaatg aggaaggtga tgagttggaa |
| | | taggccacat ctcatcagat tgacaaatcc |
| | | tttgatgatg catagggttg agacaatgat |
| | | taaggcgaca ttgaacacct cctgcaggac |
| | | ttcgggtata gactggatca aagtcacaac |
| | | ttgtcccatt ttggggttgt ttgcacttcc |
| | | tccgagtcag tgaagaagtg aacgtacagc |
| | | gtgatctaga atcgcctagg atccactgtg cg |
| 15 | S-GP/GFPnat | gcgcaccggg gatcctaggc ataccttgga |
| | | cgcgcatatt acttgatcaa agatgggaca |
| | | agttgtgact ttgatccagt ctatacccga |
| | | agtcctgcag gaggtgttca atgtcgcctt |
| | | aatcattgtc tcaaccctat gcatcatcaa |
| | | aggatttgtc aatctgatga gatgtggcct |
| | | attccaactc atcaccttcc tcattttggc |
| | | tggcagaagt tgtgatggca tgatgattga |
| | | taggaggcac aatctcaccc acgttgagtt |
| | | caacctcaca agaatgtttg acaacttgcc |
| | | acaatcatgt agcaagaaca acacacatca |
| | | ttactacaaa ggaccatcta acacaacatg |
| | | gggaattgaa ctcactttga caaacacatc |
| | | cattgcaaat gaaactactg gaaacttttc |
| | | caacatcaga agccttgcat atggtaacat |
| | | tagtaattgt gataagacag aagaagcagg |
| | | tcacacatta aaatggttgc ttaatgagtt |
| | | acacttcaat gtgctccatg tcactcgtca |
| | | tgtaggtgcc agatgcaaaa cagttgaggg |
| | | tgctggggtg ttgatccagt acaacttgac |
| | | agttggggat agaggaggtg aggttggcag |
| | | acatcttatt gcgtcgcttg ctcaaatcat |
| | | tggggaccca aaaattgcgt gggttggaaa |
| | | atgtttcaat aactgtagtg gagggtcttg |
| | | cagactaaca aactgtgaag gtgggacaca |
| | | ttacaatttc ctgatcatac agaacaccac |
| | | atgggaaaat cactgtacat atactccaat |
| | | ggcaacaata aggatggctc tccaaaaaac |
| | | tgcttatagt tctgtgagca ggaaactcct |
| | | tggctttttc acttgggact tgagtgactc |
| | | tactgggcaa catgtcccag gtggttactg |
| | | tttggagcaa tgggctattg tttgggctgg |
| | | aataaaatgt tttgataaca ctgtgatggc |
| | | aaaatgcaac aaagatcaca atgaagaatt |
| | | ttgcgatacg atgaggttat ttgatttcaa |
| | | tcagaatgct atcaaaacct tacaacttaa |
| | | tgttgagaat tcgttgaatc tctttaaaaa |
| | | gactatcaac ggacttattt ctgactcact |
| | | tgtgattaga aacagtctca aacagcttgc |
| | | caaaatccct tattgcaact atacaaaatt |
| | | ttggtacatc aatgatacca tcacagggag |
| | | acattcttta ccgcagtgtt ggttagttca |
| | | caatggctcg tacctcaatg aaacgcattt |
| | | taagaatgat tggttgtggg agagccagaa |
| | | tctgtacaat gaaatgctga taaaagaata |
| | | tgaagaaaga caaggtaaga ctccactagc |
| | | attgacagac atttgcttct ggtctttggt |
| | | gttttacacc atcacagtgt ttctccactt |
| | | agttggaata cccactcata ggcacatcat |
| | | tggtgatggc tgtccgaagc cacataggat |
| | | tactaggaac tctctttgca gctgtgggta |
| | | ttataaaatc ccaaagaaac cctacaaatg |
| | | ggtgagactg ggtaaataag ccctagcctc |
| | | gacatgggcc tcgacgtcac tccccaatag |
| | | gggagtgacg tcgaggcctc tgaggacttg |
| | | agcatgtctt cttacttgta cagctcgtcc |
| | | atgccgagag tgatcccggc ggcggtcacg |
| | | aactccagca ggaccatgtg atcgcgcttc |
| | | tcgttggggt ctttgctcag ggcggactgg |
| | | gtgctcaggt agtggttgtc gggcagcagc |
| | | acggggccgt cgccgatggg ggtgttctgc |
| | | tggtagtggt cggcgagctg cacgctgccg |
| | | tcctcgatgt tgtggcgggt cttgaagttc |
| | | accttgatgc cgttcttctg cttgtcggcg |
| | | gtgatataga ccttgtggct gttgtagttg |
| | | tactccagct tgtgccccag gatgttgccg |
| | | tcctccttga agtcgatgcc cttcagctcg |
| | | atgcggttca ccagggtgtc gccctcgaac |
| | | ttcacctcgg cgcgggtctt gtagttgccg |
| | | tcgtccttga agaagatggt gcgctcctgg |
| | | acgtagcctt cgggcatggc ggacttgaag |
| | | aagtcgtgct gcttcatgtg gtcggggtag |
| | | cggacgaagc actgcacgcc gtaggtcaag |
| | | gtggtcacga gggtgggcca gggcacgggc |
| | | agcttgccgg tggtgcagat gaacttcagg |
| | | gtcagcttgc cgtaggtggc atcgccctcg |
| | | ccctcgccgg acacgctgaa cttgtggccg |
| | | tttacgtcgc cgtccagctc gaccaggatg |
| | | ggcaccaccc cggtgaacag ctcctcgccc |
| | | ttgctcacca tgaagacatt ttggggttgt |
| | | ttgcacttcc tccgagtcag tgaagaagtg |
| | | aacgtacagc gtgatctaga atcgcctagg |
| | | atccactgtg cg |
| 16 | SΔBbsI- | gcgcaccggg gatcctaggc ataccttgga |
| | Pichinde virus | cgcgcatatt acttgatcaa agatgggaca |
| | strain Munchique | agttgtgact ttgatccagt ctatacccga |
| | CoAn4763 isolate P18 | agtcctgcag gaggtgttca atgtcgcctt |
| | (Genbank accession | aatcattgtc tcaaccctat gcatcatcaa |
| | number EF529746.1) | aggatttgtc aatctgatga gatgtggcct |
| | segment S, wherein | attccaactc atcaccttcc tcattttggc |
| | non-coding mutation | tggcagaagt tgtgatggca tgatgattga |
| | were introduced to | taggaggcac aatctcaccc acgttgagtt |
| | delete four BbsI | caacctcaca agaatgtttg acaacttgcc |
| | restriction sites. | acaatcatgt agcaagaaca acacacatca |
| | The genomic segment | ttactacaaa ggaccatcta acacaacatg |
| | is RNA, the sequence | gggaattgaa ctcactttga caaacacatc |
| | in SEQ ID NO: 16 is | cattgcaaat gaaactactg gaaacttttc |
| | shown for DNA; | caacatcaga agccttgcat atggtaacat |
| | however, exchanging | tagtaattgt gataagacag aagaagcagg |
| | all thymidines (“T”) | tcacacatta aaatggttgc ttaatgagtt |
| | in SEQ ID NO: 1 for | acacttcaat gtgctccatg tcactcgtca |
| | uridines (“U”) | tgtaggtgcc agatgcaaaa cagttgaggg |
| | provides the RNA | tgctggggtg ttgatccagt acaacttgac |
| | sequence. | agttggggat agaggaggtg aggttggcag |
| | | acatcttatt gcgtcgcttg ctcaaatcat |
| | | tggggaccca aaaattgcgt gggttggaaa |
| | | atgtttcaat aactgtagtg gagggtcttg |
| | | cagactaaca aactgtgaag gtgggacaca |
| | | ttacaatttc ctgatcatac agaacaccac |
| | | atgggaaaat cactgtacat atactccaat |
| | | ggcaacaata aggatggctc tccaaaaaac |
| | | tgcttatagt tctgtgagca ggaaactcct |
| | | tggctttttc acttgggact tgagtgactc |
| | | tactgggcaa catgtcccag gtggttactg |
| | | tttggagcaa tgggctattg tttgggctgg |
| | | aataaaatgt tttgataaca ctgtgatggc |
| | | aaaatgcaac aaagatcaca atgaagaatt |
| | | ttgcgatacg atgaggttat ttgatttcaa |
| | | tcagaatgct atcaaaacct tacaacttaa |
| | | tgttgagaat tcgttgaatc tctttaaaaa |
| | | gactatcaac ggacttattt ctgactcact |
| | | tgtgattaga aacagtctca aacagcttgc |
| | | caaaatccct tattgcaact atacaaaatt |
| | | ttggtacatc aatgatacca tcacagggag |
| | | acattcttta ccgcagtgtt ggttagttca |
| | | caatggctcg tacctcaatg aaacgcattt |
| | | taagaatgat tggttgtggg agagccagaa |
| | | tctgtacaat gaaatgctga taaaagaata |
| | | tgaagaaaga caaggtaaga ctccactagc |
| | | attgacagac atttgcttct ggtctttggt |
| | | gttttacacc atcacagtgt ttctccactt |
| | | agttggaata cccactcata ggcacatcat |
| | | tggtgatggc tgtccgaagc cacataggat |
| | | tactaggaac tctctttgca gctgtgggta |
| | | ttataaaatc ccaaagaaac cctacaaatg |
| | | ggtgagactg ggtaaataag ccctagcctc |
| | | gacatgggcc tcgacgtcac tccccaatag |
| | | gggagtgacg tcgaggcctc tgaggacttg |
| | | agctcagagg ttgatcagat ctgtgttgtt |
| | | cctgtacagc gtgtcaatag gcaagcatct |
| | | catcggcttc tggtccctaa cccagcctgt |
| | | cactgttgca tcaaacatga tggtatcaag |
| | | caatgcacag tgaggattcg cagtggtttg |
| | | tgcagccccc ttcttcttct tctttatgac |
| | | caaaccttta tgtttggtgc agagtagatt |
| | | gtatctctcc cagatctcat cctcaaaggt |
| | | gcgtgcttgc tcggcactga gtttcacgtc |
| | | aagcactttt aagtctcttc tcccatgcat |
| | | ttcgaacaaa ctgattatat catctgaacc |
| | | ttgagcagtg aaaaccatgt tttgaggtaa |
| | | atgtctgatg attgaggaaa tcaggcctgg |
| | | ttgggcatca gccaagtcct ttaaaaggag |
| | | accatgtgag tacttgcttt gctctttgaa |
| | | ggacttctca tcgtggggaa atctgtaaca |
| | | atgtatgtag ttgcccgtgt caggctggta |
| | | gatggccatt tccaccggat catttggtgt |
| | | tccttcaatg tcaatccatg tggtagcttt |
| | | tgaatcaagc atctgaattg aggacacaac |
| | | agtatcttct ttctccttag ggatttgttt |
| | | aaggtccggt gatcctccgt ttcttactgg |
| | | tggctggata gcactcggct tcgaatctaa |
| | | atctacagtg gtgttatccc aagccctccc |
| | | ttgaacttga gaccttgagc caatgtaagg |
| | | ccaaccatcc cctgaaagac aaatcttgta |
| | | tagtaaattt tcataaggat ttctctgtcc |
| | | gggtgtagtg ctcacaaaca taccttcacg |
| | | attctttatt tgcaatagac tctttatgag |
| | | agtactaaac atagaaggct tcacctggat |
| | | ggtctcaagc atattgccac catcaatcat |
| | | gcaagcagct gctttgactg ctgcagacaa |
| | | actgagattg taccctgaga tgtttatggc |
| | | tgatggctca ttactaatga tttttagggc |
| | | actgtgttgc tgtgtgagtt tctctagatc |
| | | tgtcatgttc gggaacttga cagtgtagag |
| | | caaaccaagt gcactcagcg cttggacaac |
| | | atcattaagt tgttcacccc cttgctcagt |
| | | catacaagcg atggttaagg ctggcattga |
| | | tccaaattga ttgatcaaca atgtattatc |
| | | cttgatgtcc cagatcttca caaccccatc |
| | | tctgttgcct gtgggtctag cattagcgaa |
| | | ccccattgag cgaaggattt cggctctttg |
| | | ttccaactga gtgtttgtga gattgccccc |
| | | ataaacacca ggctgagaca aactctcagt |
| | | tctagtgact ttctttctta acttgtccaa |
| | | atcagatgca agctccatta gctcctcttt |
| | | ggctaagcct cccaccttaa gcacattgtc |
| | | cctctggatt gatctcatat tcatcagagc |
| | | atcaacctct ttgttcatgt ctcttaactt |
| | | ggtcagatca gaatcagtcc ttttatcttt |
| | | gcgcatcatt ctttgaactt gagcaacttt |
| | | gtgaaagtca agagcagata acagtgctct |
| | | tgtgtccgac aacacatcag ccttcacagg |
| | | atgggtccag ttggatagac ccctcctaag |
| | | ggactgtacc cagcggaatg atgggatgtt |
| | | gtcagacatt ttggggttgt ttgcacttcc |
| | | tccgagtcag tgaagaagtg aacgtacagc |
| | | gtgatctaga atcgcctagg atccactgtg cg |

What is claimed:

1. A Pichinde virus genomic segment, wherein the genomic segment is engineered to carry a viral open reading frame (“ORF”) in a position other than the wild-type position of the ORF, wherein the Pichinde virus genomic segment is selected from the group consisting of:
(i) an S segment, wherein the ORF encoding the nucleoprotein (“NP”) is under control of a Pichinde virus 5′ untranslated region (“UTR”);
(ii) an S segment, wherein the ORF encoding the matrix protein Z (“Z protein”) is under control of a Pichinde virus 5′ UTR;
(iii) an S segment, wherein the ORF encoding the RNA dependent RNA polymerase L (“L protein”) is under control of a Pichinde virus 5′ UTR;
(iv) an S segment, wherein the ORF encoding the viral glycoprotein (“GP”) is under control of a Pichinde virus 3′ UTR;
(v) an S segment, wherein the ORF encoding the L protein is under control of a Pichinde virus 3′ UTR;
(vi) an S segment, wherein the ORF encoding the Z protein is under control of a Pichinde virus 3′ UTR;
(vii) an L segment, wherein the ORF encoding the GP is under control of a Pichinde virus 5′ UTR;
(viii) an L segment, wherein the ORF encoding the NP is under control of a Pichinde virus 5′ UTR;
(ix) an L segment, wherein the ORF encoding the L protein is under control of a Pichinde virus 5′ UTR;
(x) an L segment, wherein the ORF encoding the GP is under control of a Pichinde virus 3′ UTR;
(xi) an L segment, wherein the ORF encoding the NP is under control of a Pichinde virus 3′ UTR; and
(xii) an L segment, wherein the ORF encoding the Z protein is under control of a Pichinde virus 3′ UTR.

2. The Pichinde virus genomic segment of claim 1, wherein the Pichinde virus 3′ UTR is the 3′ UTR of the Pichinde virus S segment or the Pichinde virus L segment, and wherein the Pichinde virus 5′ UTR is the 5′ UTR of the Pichinde virus S segment or the Pichinde virus L segment.

3. A cDNA of the Pichinde virus genomic segment of claim 1.

4. A DNA expression vector comprising the cDNA of claim 3.

5. A host cell comprising the Pichinde virus genomic segment of claim 1, the cDNA of claim 3, or the vector of claim 4.

6. A Pichinde virus particle comprising the Pichinde virus genomic segment of claim 1 and a second Pichinde virus genomic segment so that the Pichinde virus particle comprises an S segment and an L segment.

7. The Pichinde virus particle of claim 6, wherein the Pichinde virus particle is infectious and replication competent.

8. The Pichinde virus particle of claim 6, wherein the Pichinde virus particle is attenuated.

9. The Pichinde virus particle of claim 6, wherein the Pichinde virus particle is infectious but unable to produce further infectious progeny in non-complementing cells.

10. The Pichinde virus particle of claim 9, wherein at least one of the four ORFs encoding GP, NP, Z protein, and L protein is removed or functionally inactivated.

11. The Pichinde virus particle of claim 9, wherein at least one of the four ORFs encoding GP, NP, Z protein, and L protein is removed and replaced with a heterologous ORF from an organism other than a Pichinde virus.

12. The Pichinde virus particle of claim 9, wherein only one of the four ORFs encoding GP, NP, Z protein and L protein is removed and replaced with a heterologous ORF from an organism other than a Pichinde virus.

13. The Pichinde virus particle of claim 9, wherein the ORF encoding GP is removed and replaced with a heterologous ORF from an organism other than a Pichinde virus.

14. The Pichinde virus particle of claim 9, wherein the ORF encoding NP is removed and replaced with a heterologous ORF from an organism other than a Pichinde virus.

15. The Pichinde virus particle of claim 9, wherein the ORF encoding the Z protein is removed and replaced with a heterologous ORF from an organism other than a Pichinde virus.

16. The Pichinde virus particle of claim 9, wherein the ORF encoding the L protein is removed and replaced with a heterologous ORF from an organism other than a Pichinde virus.

17. The Pichinde virus particle of anyone of claims 11 to 16, wherein the heterologous ORF encodes a reporter protein.

18. The Pichinde virus particle of anyone of claims 11 to 16, wherein the heterologous ORF encodes an antigen derived from an infectious organism, tumor, or allergen.

19. The Pichinde virus particle of claim 18, wherein the heterologous ORF encoding an antigen is selected from human immunodeficiency virus antigens, hepatitis C virus antigens, varizella zoster virus antigens, cytomegalovirus antigens, mycobacterium tuberculosis antigens, tumor associated antigens, and tumor specific antigens (such as tumor neoantigens and tumor neoepitopes).

20. The Pichinde virus particle of anyone of claims 11 to 18, wherein the growth or infectivity of the Pichinde virus particle is not affected by the heterologous ORF from an organism other than a Pichinde virus.

21. A method of producing the Pichinde virus genomic segment of claim 1, wherein said method comprises transcribing the cDNA of claim 3.

22. A method of generating the Pichinde virus particle of claim 6, wherein the method comprises:
(i) transfecting into a host cell the cDNA of claim 3;
(ii) transfecting into the host cell a plasmid comprising the cDNA of the second Pichinde virus genomic segment;
(iii) maintaining the host cell under conditions suitable for virus formation; and
(iv) harvesting the Pichinde virus particle.

23. The method of claim 22, wherein the transcription of the L segment and the S segment is performed using a bidirectional promoter.

24. The method of claim 22, wherein the method further comprises transfecting into a host cell one or more nucleic acids encoding a Pichinde virus polymerase.

25. The method of claim 24, wherein the Pichinde virus polymerase is the L protein.

26. The method of claim 22 or 24, wherein the method further comprises transfecting into the host cell one or more nucleic acids encoding the NP protein.

27. The method of claim 22, wherein transcription of the L segment, and the S segment are each under the control of a promoter selected from the group consisting of:
(i) a RNA polymerase I promoter;
(ii) a RNA polymerase II promoter; and
(iii) a T7 promoter.

28. A vaccine comprising the Pichinde virus particle of claims 6 to 19 and a pharmaceutically acceptable carrier.

29. A pharmaceutical composition comprising a Pichinde virus particle of claims 6 to 19 and a pharmaceutically acceptable carrier.

30. The Pichinde virus genomic segment of claim 1 or the Pichinde virus particle of claim 6, wherein the Pichinde virus genomic segment or Pichinde virus particle is derived from the strain Munchique CoAn4763 isolate P18, or P2 strain.

31. A tri-segmented Pichinde virus particle comprising one L segment and two S segments, wherein propagation of the tri-segmented Pichinde virus particle does not result in a replication-competent bi-segmented viral particle after 70 days of persistent infection in mice lacking type I interferon receptor, type II interferon receptor and recombination activating gene 1 (RAG1) and having been infected with 10⁴ PFU of the tri-segmented Pichinde virus particle.

32. The tri-segmented Pichinde virus particle of claim 31, wherein inter-segmental recombination of the two S segments, uniting two Pichinde virus ORFs on only one instead of two separate segments, abrogates viral promoter activity.

33. A tri-segmented Pichinde virus particle comprising two L segments and one S segment, wherein propagation of the tri-segmented Pichinde virus particle does not result in a replication-competent bi-segmented viral particle after 70 days of persistent infection in mice lacking type I interferon receptor, type II interferon receptor and recombination activating gene 1 (RAG1) and having been infected with 10⁴ PFU of the tri-segmented Pichinde virus particle.

34. A tri-segmented Pichinde virus particle of claim 33, wherein, inter-segmental recombination of the two L segments, uniting two Pichinde virus ORFs on only one instead of two separate segments, abrogates viral promoter activity.

35. The tri-segmented Pichinde virus particle of claim 31, wherein one of the two S segments is selected from the group consisting of:
(i) an S segment, wherein the ORF encoding the NP is under control of a Pichinde virus 5′ UTR;
(ii) an S segment, wherein the ORF encoding the Z protein is under control of a Pichinde virus 5′ UTR;
(iii) an S segment, wherein the ORF encoding the L protein is under control of a Pichinde virus 5′ UTR;
(iv) an S segment, wherein the ORF encoding the GP is under control of a Pichinde virus 3′ UTR;
(v) an S segment, wherein the ORF encoding the L is under control of a Pichinde virus 3′ UTR; and
(vi) an S segment, wherein the ORF encoding the Z protein is under control of a Pichinde virus 3′ UTR.

36. The tri-segmented Pichinde virus particle of claim 33, wherein one of the two L segments is selected from the group consisting of:
(i) an L segment, wherein the ORF encoding the GP is under control of a Pichinde virus 5′ UTR;
(ii) an L segment, wherein the ORF encoding the NP is under control of a Pichinde virus 5′ UTR;
(iii) an L segment, wherein the ORF encoding the L protein is under control of a Pichinde virus 5′ UTR;
(iv) an L segment, wherein the ORF encoding the GP is under control of a Pichinde virus 3′ UTR;
(v) an L segment, wherein the ORF encoding the NP is under control of a Pichinde virus 3′ UTR; and
(vi) an L segment, wherein the ORF encoding the Z protein is under control of a Pichinde virus 3′ UTR.

37. The tri-segmented Pichinde virus particle of claim 35 or 36, wherein the Pichinde virus 3′ UTR is the 3′ UTR of the Pichinde virus S segment or the Pichinde virus L segment, and wherein the Pichinde virus 5′ UTR is the 5′ UTR of the Pichinde virus S segment or the Pichinde virus L segment.

38. The tri-segmented Pichinde virus particle of claim 31, wherein the two S segments comprise (i) one or two heterologous ORFs from an organism other than a Pichinde virus; or (ii) one or two duplicated Pichinde virus ORFs; or (iii) one heterologous ORF from an organism other than a Pichinde virus and one duplicated Pichinde virus ORF.

39. The tri-segmented Pichinde virus particle of claim 33, wherein the two L segments comprise (i) one or two heterologous ORFs from an organism other than a Pichinde virus; or (ii) two duplicated Pichinde virus ORFs; or (iii) one heterologous ORF from an organism other than a Pichinde virus and one duplicated Pichinde virus ORF.

40. The tri-segmented Pichinde virus particle of claim 38 or 39, wherein the heterologous ORF encodes an antigen derived from an infectious organism, tumor, or allergen.

41. The tri-segmented Pichinde virus particle of claim 40, wherein the heterologous ORF encoding an antigen is selected from human immunodeficiency virus antigens, hepatitis C virus antigens, varizella zoster virus antigens, cytomegalovirus antigens, mycobacterium tuberculosis antigens, tumor associated antigens, and tumor specific antigens (such as tumor neoantigens and tumor neoepitopes).

42. The tri-segmented Pichinde virus particle of claim 38 or 39, wherein at least one heterologous ORF encodes a fluorescent protein.

43. The tri-segmented Pichinde virus particle of claim 42, wherein the fluorescent protein is green fluorescent protein or red fluorescent protein.

44. The tri-segmented Pichinde virus particle of any one of claims 31 to 41, wherein the tri-segmented Pichinde virus particle comprises all four Pichinde virus ORFs, and wherein the tri-segmented Pichinde virus particle is infectious and replication competent.

45. The tri-segmented Pichinde virus particle of any one of claims 31 to 43, wherein the tri-segmented Pichinde virus particle lacks one or more of the four Pichinde virus ORFs, wherein the tri-segmented Pichinde virus particle is infectious but unable to produce further infectious progeny in non-complementing cells.

46. The tri-segmented Pichinde virus particle of any one of claims 31 to 43, wherein the tri-segmented Pichinde virus particle lacks one of the four Pichinde virus ORFs, wherein the tri-segmented Pichinde virus particle is infectious but unable to produce further infectious progeny in non-complementing cells.

47. The tri-segmented Pichinde virus particle of claim 44 or 45, wherein the Pichinde virus lacks the GP ORF.

48. A tri-segmented Pichinde virus particle comprising one L segment and two S segments, wherein a first S segment is engineered to carry an ORF encoding GP in a position under control of a Pichinde virus 3′ UTR and an ORF encoding a first gene of interest in a position under control of a Pichinde virus 5′ UTR and a second S segment is engineered to carry an ORF encoding NP in a position under control of a Pichinde virus 3′ UTR and an ORF encoding a second gene of interest in a position under control of a Pichinde virus 5′ UTR.

49. A tri-segmented Pichinde virus particle comprising one L segment and two S segments, wherein a first S segment is engineered to carry an ORF encoding GP in a position under control of a Pichinde virus 5′ UTR and an ORF encoding a first gene of interest in a position under control of a Pichinde virus 3′ UTR and a second S segment is engineered to carry an ORF encoding NP in a position under control of a Pichinde virus 5′ UTR and an ORF encoding a second gene of interest in a position under control of a Pichinde virus 3′ UTR.

50. The tri-segmented Pichinde virus particle of claim 48 or 49, wherein the gene of interest encodes an antigen derived from an infectious organism, tumor, or allergen.

51. The tri-segmented Pichinde virus particle of claim 50, wherein the gene of interest encodes an antigen selected from human immunodeficiency virus antigens, hepatitis C virus antigens, varizella zoster virus antigens, cytomegalovirus antigens, mycobacterium tuberculosis antigens, tumor associated antigens, and tumor specific antigens (such as tumor neoantigens and tumor neoepitopes).

52. The tri-segmented Pichinde virus particle of claim 48 or 49, wherein at least one gene of interest encodes a fluorescent protein.

53. The tri-segmented Pichinde virus particle of claim 52, wherein the fluorescent protein is green fluorescent protein or red fluorescent protein.

54. A cDNA of the tri-segmented Pichinde virus particle genome of any one of claim 31, 33, 35, 36, 48 or 49.

55. A DNA expression vector comprising the cDNA of claim 54.

56. A host cell comprising the tri-segmented Pichinde virus particle of claim 31 or 33, the cDNA of claim 54, or the vector of claim 55.

57. The tri-segmented Pichinde virus particle of any one of claims 31 to 49, wherein the tri-segmented Pichinde virus particle is attenuated.

58. A method of generating the tri-segmented Pichinde virus particle of claim 31, wherein the method comprises:
(i) transfecting into a host cell one or more cDNAs of the L segment and two S segments;
(ii) maintaining the host cell under conditions suitable for virus formation; and
(iii) harvesting the Pichinde virus particle.

59. A method of generating the tri-segmented Pichinde virus particle of claim 33, wherein the method comprises:
(i) transfecting into a host cell one or more cDNAs of two L segments and one S segment;
(ii) maintaining the host cell under conditions suitable for virus formation; and
(iii) harvesting the Pichinde virus particle.

60. The method of claim 58, wherein the transcription of one L segment and two S segments is performed using a bidirectional promoter.

61. The method of claim 59, wherein the transcription of two L segments and one S segment is performed using a bidirectional promoter.

62. The method of claim 58 or 59, wherein the method further comprises transfecting into the host cell one or more nucleic acids encoding a Pichinde virus polymerase.

63. The method of claim 62, wherein the Pichinde virus polymerase is the L protein.

64. The method of claim 58, 59, 60 or 61, wherein the method further comprises transfecting into the host cell one or more nucleic acids encoding the NP protein.

65. The method of claim 58, wherein transcription of the L segment, and the two S segments are each under the control of a promoter selected from the group consisting of:
(i) a RNA polymerase I promoter;
(ii) a RNA polymerase II promoter; and
(iii) a T7 promoter.

66. The method of claim 59, wherein transcription of two L segments, and the S segment are each under the control of a promoter selected from the group consisting of:
(i) a RNA polymerase I promoter;
(ii) a RNA polymerase II promoter; and
(iii) a T7 promoter.

67. The tri-segmented Pichinde virus particle of any one of claims 31 to 49, wherein the tri-segmented Pichinde virus particle has the same tropism as the bi-segmented Pichinde virus particle.

68. The tri-segmented Pichinde virus particle of any one of claims 31 to 49, wherein the tri-segmented Pichinde virus particle is replication deficient.

69. A vaccine comprising a tri-segmented Pichinde virus particle of any one of claim 31 to 49, 67 or 68 and a pharmaceutically acceptable carrier.

70. A pharmaceutical composition comprising a tri-segmented Pichinde virus particle of any one of the claim 31 to 49, 67 or 68 and a pharmaceutically acceptable carrier.

71. The tri-segmented Pichinde virus particle of any one of claim 31 to 49, 67 or 68, wherein the Pichinde virus is strain Munchique CoAn4763 isolate P18, or P2 strain.


SEQUENCE LISTING (16 sequences)

SEQ ID NO: 1 - DNA, 3422 bp, Artificial Sequence
  Pichinde virus strain Munchique CoAn4763 isolate P18 (Genbank accession number EF529746.1) segment S, complete sequence
gcgcaccggggatcctaggcataccttggacgcgcatattacttgatcaaagatgggacaagttgtgactttgatccagtctatacccgaagtcctgcaggaggtcttcaatgtcgccttaatcattgtctcaaccctatgcatcatcaaaggatttgtcaatctgatgagatgtggcctattccaactcatcaccttcctcattttggctggcagaagttgtgatggcatgatgattgataggaggcacaatctcacccacgttgagttcaacctcacaagaatgtttgacaacttgccacaatcatgtagcaagaacaacacacatcattactacaaaggaccatctaacacaacatggggaattgaactcactttgacaaacacatccattgcaaatgaaactactggaaacttttccaacatcagaagccttgcatatggtaacattagtaattgtgataagacagaagaagcaggtcacacattaaaatggttgcttaatgagttacacttcaatgtgctccatgtcactcgtcatgtaggtgccagatgcaaaacagttgagggtgctggggtgttgatccagtacaacttgacagttggggatagaggaggtgaggttggcagacatcttattgcgtcgcttgctcaaatcattggggacccaaaaattgcgtgggttggaaaatgtttcaataactgtagtggagggtcttgcagactaacaaactgtgaaggtgggacacattacaatttcctgatcatacagaacaccacatgggaaaatcactgtacatatactccaatggcaacaataaggatggctctccaaaaaactgcttatagttctgtgagcaggaaactccttggctttttcacttgggacttgagtgactctactgggcaacatgtcccaggtggttactgtttggagcaatgggctattgtttgggctggaataaaatgttttgataacactgtgatggcaaaatgcaacaaagatcacaatgaagaattttgcgatacgatgaggttatttgatttcaatcagaatgctatcaaaaccttacaacttaatgttgagaattcgttgaatctctttaaaaagactatcaacggacttatttctgactcacttgtgattagaaacagtctcaaacagcttgccaaaatcccttattgcaactatacaaaattttggtacatcaatgataccatcacaggaagacattctttaccgcagtgttggttagttcacaatggctcgtacctcaatgaaacgcattttaagaatgattggttgtgggagagccagaatctgtacaatgaaatgctgataaaagaatatgaagaaagacaaggtaagactccactagcattgacagacatttgcttctggtctttggtgttttacaccatcacagtgtttctccacttagttggaatacccactcataggcacatcattggtgatggctgtccgaagccacataggattactaggaactctctttgcagctgtgggtattataaaatcccaaagaaaccctacaaatgggtgagactgggtaaataagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaagaagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtgtcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 2 - DNA, 7058 bp, Artificial Sequence
  PIC-L-seg Pichinde virus strain Munchique CoAn4763 isolate P18 (Genbank accession number EF529747.1) segment L, complete sequence with non-coding mutations introduced  to delete BsmBI restriction sites
gcgcaccggggatcctaggcatctttgggtcacgcttcaaatttgtccaatttgaacccagctcaagtcctggtcaaaacttgggatgggactcagatatagcaaagaggtcaggaagagacatggcgacgaagatgtggtgggaagggtccccatgaccctcaatctaccacagggcctgtatggcaggttcaactgcaaatcttgctggttcgtcaacaaaggtctcatcaggtgcaaagaccactatctgtgtcttgggtgcttaaccaaaatgcactccagaggcaatctctgcgagatatgcggccactcactgccaaccaagatggagttcctagaaagcccctctgcaccaccctacgagccataaaccagggcccctgggcgcacccccctccgggggtgcgcccgggggcccccggccccatggggccggttgtttactcgatctccactgactcattgtcctcaaacaactttcgacacctgattcccttgatcttgaagggtcctgtctcgtctgcaatcataacagatcctagagtcttacttcttattatactaaagtgaccacaattcaaccaatctttggcatcatgcaacatgtgttcaaacacttcggggaaattttcaatcatgagtcttaaatcctgctcgttcatacttattcccttgttgtgagactgtgcacttgaaaggtactgaaaaaggttggcaataaatcttggccttttctcaggttctaatgcttccagtgcaatgatgaccacctttgagtctaagttcacttccaatctagaaaccactctgttgccctctttgatcaacccaccctctaaaatgaggggttgcatcccaacatcaggaccaatcaacttataggaaaatttgtttttcaaatccttgaaacgatttttcaaatctattctcaccttctggaacacagttgaccttgacttgaagtgaatgtcttgaccttccaatagatcattgaagtctagaacatcttttccgttgatgagaggattcagaaccaaaagtgacacaccatccagacttatgtgattcccggaagattgagaaacataatactcaacagaatgggggttcaacaataggtaaccatcagagtccaatgagtccagcaatgactccctttcaataagaaatcttaattttaatatgtaattggtagacctctcatatctaaatttgtggctcactctcttatgagaaaatgttaggttgagctcaatgggaatgacctcagaaggtgatgctaaaatgagttgttcaatgttctcatagttatctctattcacccagtcaagttcattaataaatacactaatgttcaaattaacacaggacaaaatcagtttgctgcttacaaagccaacatccaagtcatccagattcattgtcctagaagtgttattctttttgcagtcacaaatgaactgggttaattgtttcagatcatgttgtgcattgtttggcaacaattcaagctcaccaaaccaaaaatatttcttgaactgagatgttgacataatcacaggcaccaacattgactcaaacaaaatctgtatcaagaaatttgtgcacacttcttctggttcaaggttgaatcctctctccagtggatgagactctctgctatgggacattgcaagctcattttgctttacaatatacaattcttctctgcgatgttttataatatgactaacaataccaagacattctgatgttatatcaattgccacacaaaggtctaagaactttatcctctgaacccatgatagcctcagcatattcaaatcagacaggaaaggggatatgtgttcatcaaatagtgtagggaagttcctcctgattgagtaaagtatgtggttgatgcccaccttgtcctcaagctcagaatgtgtgcttggttttattggccagaagtgattgggattgtttaggtgagtgactatcttgggtacttcagctttttgaaacacccagttacccaactcgcaagcattggttaacacaagagcaaaataatcccaaattaagggtctggagtactcacttacttcaccaagtgctgctttacaataaacacctttgcgctgattacaaaagtgacaatcacggtgtaagataatcttgcttgtaatatccctgatatacttaaatcctcctttcccatctcttacacattttgagcccatacttttgcaaactcctatgaatcctgatgctatgctgctctgaaaagctgatttgttgatagcatcagccaaaatcttcttagcccctctgacatagttctttgataatttggactgtacggatttgacaagactgggtatttcttctcgctgcacagttcttgttgtgctcattaacttagtacgaagcaccaatctgagatcaccatgaacccttaaatttaaccacctaatattaagagcatcctcaatagcctcagtctcgacatcacaagtctctaataactgttttaagcagtcatccggtgattgctgaagagttgttacaatataactttcttccagggctccagactgtattttgtaaaatattttcctgcatgcctttctgattattgaaagtagcagatcatcaggaaatagtgtctcaattgatcgctgaagtctgtaccctctcgacccattaacccaatcgagtacatccatttcttccaggcacaaaaatggatcatttggaaacccactatagattatcatgctatttgttcgttttgcaatggcccctacaacctctattgacaccccgttagcaacacattggtccagtattgtgtcaattgtatctgcttgctgattgggtgctttagcctttatgttgtgtagagctgcagcaacaaactttgtaaggagggggacttcttgtgaccaaatgaagaatctcgatttgaactcacttgcaaaggtccccacaactgttttagggctcacaaacttgttgagtttgtctgatagaaagtagtgaaactccatacagtccaataccaattcaacattcaactcatctctgtccttaaatttgaaaccctcattcaaggataacatgatctcatcatcactcgaagtatatgagatgaaccgtgctccataacaaagctccaatgcgtaattgatgaactgctcagtgattagaccatataagtcagaggtgttgtgtaggatgccctgacccatatctaagactgaagagatgtgtgatggtaccttgcccttctcaaagtacccaaacataaattcctctgcaattgtgcacccccctttatccatcatacccaacccccttttcaagaaacctttcatgtatgcctcaacgacattgaagggcacttccaccatcttgtgaatgtgccatagcaatatgttgatgactgcagcattgggaacttctgacccatctttgagtttgaactcaagaccttttaataatgcggcaaagataaccggcgacatgtgtggcccccattttgaatggtccattgacaccgcaagaccactttgcctaacaactgacttcatgtctaataatgctctctcaaactctttctcgttgttcagacaagtatacctcatgttttgcataagggattcagagtaatcctcaatgagtctggttgtgagtttagtatttaaatcaccgacataaagctccctgttgccacccacctgttctttataagaaagaccaaatttcaatctccctacattggtggatacaccagacctctctgtgggagactcatctgaatagaaacagagatttcgtaaggatgagttggtaaaaaagctttgatccaatcttttagctatcgattcagaattgctctctcttgagcttatacgtgatgtctctctaatttgtagtgctgcatctgtgaacccaagtctgcttctacttttgtgatcatatcttccgactcgattatcataatcgcttgcaatgagaatgtatttaaagcactcaaaataatcagcttctttgtacgccttcaatgtgaggttctttattaaaaactccagaggacacggattcattagtctgtctgcaaagtacactgatctagcagtgacatcctcatagatcaagtttacaagatcctcatacacttctgctgaaaacaggctgtaatcaaaatcctttacatcatgaagtgaagtctctcttttgatgacaaccattgtcgatttgggccataatctctctagtggacatgaagtcttaaggttggttttgacattggtgtcaaccttagacaatacttttgcaactctggtctcaatttctttaagacagtcaccctgatcttctgatagtaactcttcaactccatcaggctctattgactccttttttatttggatcaatgatgacaacctcttcagaatcttgaaatttacctcctttggatctaacttgtatttacccttagttttgaaatgttcaatcatttccacaacaacagcagacacaatggaagagtaatcatattcagtgatgacctcaccaacttcattgagttttggaaccaccacacttttgttgctggacatatccaaggctgtacttgtgaaggagggagtcatagggtcacaaggaagcaggggtttcacttccaatgagctactgttaaatagtgatagacaaacactaagtacatccttattcaaccccggccttccctcacatttggattccagctttttaccaagtagtctctctatatcatgcaccatcttctcttcttcctcagtaggaagttccatactattagaagggttgaccaagactgaatcaaactttaactttggttccaagaacttctcaaaacatttgatttgatcagttaatctatcaggggtttctttggttataaaatggcataaataggagacattcaaaacaaacttaaagatcttagccatatcttcctctctggagttgctgagtaccagaagtatcaaatcatcaataagcattgctgtctgccattctgaaggtgttagcataacgactttcaatttctcaaacaattctttaaaatgaacttcatttacaaaggccataatgtaatatctaaagccttgcaagtaaacttgaatacgcttggaaggggtgcacagtatgcagagaataagtcgtctgagtaaatcagaaacagaatccaagaggggttgggacataaagtccaaccaggataacatctccacacaagtcctttgaatcacatctgcactaaagatcggtaagaaaaatctcttgggatcacagtaaaaagacgcttttgtttcatacaaacccccacttttggatctataagcaacagcataacacctggacctctcccctgtcttctggtacagtagtgtgagagaacctccttctccaaatcgctggaagaaaacttcgtcacagtaaaccttcccataaaactcatcagcattgttcaccttcatcttaggaactgctgctgtcttcatgctattaatgagtgacaaactcaaacttgacaatgttttcagcaattcctcaaactcactttcgcccatgatggtataatcaggctgccctcttcctggcctacccccacacatacactgtgactttgtcttgtattgaagacagggtttagcaccccattcatctaacactgatgttttcagattgaagtaatattcaacatcaggttcccgtagaagagggagaatgtcatcaaggggaagttcaccacagaccgagctcagtctcttcttagccttctctaaccagttggggtttttaatgaattttttagtgatttgttccatcaggaagtcgacattaatcaacctgtcatttacagacggtaacccttgcattaggagcacctctctgaacacagcacctggagaagacttgtccaagtcacacaaaatgttgtacatgataaggtccagaaccaacatggtgttcctccttgtgttaaaaaccttttgagacttaattttgttgcatattgaaagtactctaaaatattctctgctttcagttgatgaatgcttgacctcagattgcctgagttggcctattatgcccaaaatgtgtactgagcaaaactcacataatctgatttctgatttaggtacatctttgacagaacattggataaattcatggttctgaagtctagaaatcatatcttccctatctgtagcctgcagtttcctatcgagttgaccagcaagttgcaacattttaaattgctgaaagatttccatgatttttgttctacattgatctgttgtcagtttattattaatgccagacattaatgccttttccaacctcactttgtaaggaagtcccctttcctttacagcaagtagtgactccagaccgagactctgattttctaaggatgagagggaacttataaggcgttcgtactccaactcctcaacttcttcaccagatgtccttaatccatccatgagttttaaaagcaaccaccgaagtctctctaccacccaatcaggaacaaattctacataataactggatctaccgtcaataacaggtactaaggttatgttctgtctcttgagatcagaactaagctgcaacagcttcaaaaagtcctggttgtatttcttctcaaatgcttcttgactggtcctcacaaacacttccaaaagaatgaggacatctccaaccatacagtaaccatctggtgtaacatccggcaatgtaggacatgttactctcaactccctaaggatagcattgacagtcatctttgtgttgtgtttgcaggagtgtttcttgcatgaatccacttccactagcatggacaaaagcttcaggccctctatcgtgatggccctatctttgacttgtgcaagaacgttgtttttctgttcagatagctcttcccattcgggaacccattttctgactatgtctttaagttcgaaaacgtattcctccatgatcaagaaatgcctaggatcctcggtgcg

SEQ ID NO: 3 - DNA, 6619 bp, Artificial Sequence
  L-delat-BsmBI Modified Representative cDNA of the L ORF of Pichinde virus strain Munchique CoAn4763 isolate P18 (Genbank accession number EF529747.1)
gaattccgtctctgatcatggaggaatacgttttcgaacttaaagacatagtcagaaaatgggttcccgaatgggaagagctatctgaacagaaaaacaacgttcttgcacaagtcaaagatagggccatcacgatagagggcctgaagcttttgtccatgctagtggaagtggattcatgcaagaaacactcctgcaaacacaacacaaagatgactgtcaatgctatccttagggagttgagagtaacatgtcctacattgccggatgttacaccagatggttactgtatggttggagatgtcctcattcttttggaagtgtttgtgaggaccagtcaagaagcatttgagaagaaatacaaccaggactttttgaagctgttgcagcttagttctgatctcaagagacagaacataaccttagtacctgttattgacggtagatccagttattatgtagaatttgttcctgattgggtggtagagagacttcggtggttgcttttaaaactcatggatggattaaggacatctggtgaagaagttgaggagttggagtacgaacgccttataagttccctctcatccttagaaaatcagagtctcggtctggagtcactacttgctgtaaaggaaaggggacttccttacaaagtgaggttggaaaaggcattaatgtctggcattaataataaactgacaacagatcaatgtagaacaaaaatcatggaaatctttcagcaatttaaaatgttgcaacttgctggtcaactcgataggaaactgcaggctacagatagggaagatatgatttctagacttcagaaccatgaatttatccaatgttctgtcaaagatgtacctaaatcagaaatcagattatgtgagttttgctcagtacacattttgggcataataggccaactcaggcaatctgaggtcaagcattcatcaactgaaagcagagaatattttagagtactttcaatatgcaacaaaattaagtctcaaaaggtttttaacacaaggaggaacaccatgttggttctggaccttatcatgtacaacattttgtgtgacttggacaagtcttctccaggtgctgtgttcagagaggtgctcctaatgcaagggttaccgtctgtaaatgacaggttgattaatgtcgacttcctgatggaacaaatcactaaaaaattcattaaaaaccccaactggttagagaaggctaagaagagactgagctcggtctgtggtgaacttccccttgatgacattctccctcttctacgggaacctgatgttgaatattacttcaatctgaaaacatcagtgttagatgaatggggtgctaaaccctgtcttcaatacaagacaaagtcacagtgtatgtgtgggggtaggccaggaagagggcagcctgattataccatcatgggcgaaagtgagtttgaggaattgctgaaaacattgtcaagtttgagtttgtcactcattaatagcatgaagacagcagcagttcctaagatgaaggtgaacaatgctgatgagttttatgggaaggtttactgtgacgaagttttcttccagcgatttggagaaggaggttctctcacactactgtaccagaagacaggggagaggtccaggtgttatgctgttgcttatagatccaaaagtgggggtttgtatgaaacaaaagcgtctttttactgtgatcccaagagatttttcttaccgatctttagtgcagatgtgattcaaaggacttgtgtggagatgttatcctggttggactttatgtcccaacccctcttggattctgtttctgatttactcagacgacttattctctgcatactgtgcaccccttccaagcgtattcaagtttacttgcaaggctttagatattacattatggcctttgtaaatgaagttcattttaaagaattgtttgagaaattgaaagtcgttatgctaacaccttcagaatggcagacagcaatgcttattgatgatttgatacttctggtactcagcaactccagagaggaagatatggctaagatctttaagtttgttttgaatgtctcctatttatgccattttataaccaaagaaacccctgatagattaactgatcaaatcaaatgttttgagaagttcttggaaccaaagttaaagtttgattcagtcttggtcaacccttctaatagtatggaacttcctactgaggaagaagagaagatggtgcatgatatagagagactacttggtaaaaagctggaatccaaatgtgagggaaggccggggttgaataaggatgtacttagtgtttgtctatcactatttaacagtagctcattggaagtgaaacccctgcttccttgtgaccctatgactccctccttcacaagtacagccttggatatgtccagcaacaaaagtgtggtggttccaaaactcaatgaagttggtgaggtcatcactgaatatgattactcttccattgtgtctgctgttgttgtggaaatgattgaacatttcaaaactaagggtaaatacaagttagatccaaaggaggtaaatttcaagattctgaagaggttgtcatcattgatccaaataaaaaaggagtcaatagagcctgatggagttgaagagttactatcagaagatcagggtgactgtcttaaagaaattgagaccagagttgcaaaagtattgtctaaggttgacaccaatgtcaaaaccaaccttaagacttcatgtccactagagagattatggcccaaatcgacaatggttgtcatcaaaagagagacttcacttcatgatgtaaaggattttgattacagcctgttttcagcagaagtgtatgaggatcttgtaaacttgatctatgaggatgtcactgctagatcagtgtactttgcagacagactaatgaatccgtgtcctctggagtttttaataaagaacctcacattgaaggcgtacaaagaagctgattattttgagtgctttaaatacattctcattgcaagcgattatgataatcgagtcggaagatatgatcacaaaagtagaagcagacttgggttcacagatgcagcactacaaattagagagacatcacgtataagctcaagagagagcaattctgaatcgatagctaaaagattggatcaaagcttttttaccaactcatccttacgaaatctctgtttctattcagatgagtctcccacagagaggtctggtgtatccaccaatgtagggagattgaaatttggtctttcttataaagaacaggtgggtggcaacagggagctttatgtcggtgatttaaatactaaactcacaaccagactcattgaggattactctgaatcccttatgcaaaacatgaggtatacttgtctgaacaacgagaaagagtttgagagagcattattagacatgaagtcagttgttaggcaaagtggtcttgcggtgtcaatggaccattcaaaatgggggccacacatgtcgccggttatctttgccgcattattaaaaggtcttgagttcaaactcaaagatgggtcagaagttcccaatgctgcagtcatcaacatattgctatggcacattcacaagatggtggaagtgcccttcaatgtcgttgaggcatacatgaaaggtttcttgaaaagggggttgggtatgatggataaaggggggtgcacaattgcagaggaatttatgtttgggtactttgagaagggcaaggtaccatcacacatctcttcagtcttagatatgggtcagggcatcctacacaacacctctgacttatatggtctaatcactgagcagttcatcaattacgcattggagctttgttatggagcacggttcatctcatatacttcgagtgatgatgagatcatgttatccttgaatgagggtttcaaatttaaggacagagatgagttgaatgttgaattggtattggactgtatggagtttcactactttctatcagacaaactcaacaagtttgtgagccctaaaacagttgtggggacctttgcaagtgagttcaaatcgagattcttcatttggtcacaagaagtccccctccttacaaagtttgttgctgcagctctacacaacataaaggctaaagcacccaatcagcaagcagatacaattgacacaatactggaccaatgtgttgctaacggggtgtcaatagaggttgtaggggccattgcaaaacgaacaaatagcatgataatctatagtgggtttccaaatgatccatttttgtgcctggaagaaatggatgtactcgattgggttaatgggtcgagagggtacagacttcagcgatcaattgagacactatttcctgatgatctgctactttcaataatcagaaaggcatgcaggaaaatattttacaaaatacagtctggagccctggaagaaagttatattgtaacaactcttcagcaatcaccggatgactgcttaaaacagttattagagacttgtgatgtcgagactgaggctattgaggatgctcttaatattaggtggttaaatttaagggttcatggtgatctcagattggtgcttcgtactaagttaatgagcacaacaagaactgtgcagcgagaagaaatacccagtcttgtcaaatccgtacagtccaaattatcaaagaactatgtcagaggggctaagaagattttggctgatgctatcaacaaatcagcttttcagagcagcatagcatcaggattcataggagtttgcaaaagtatgggctcaaaatgtgtaagagatgggaaaggaggatttaagtatatcagggatattacaagcaagattatcttacaccgtgattgtcacttttgtaatcagcgcaaaggtgtttattgtaaagcagcacttggtgaagtaagtgagtactccagacccttaatttgggattattttgctcttgtgttaaccaatgcttgcgagttgggtaactgggtgtttcaaaaagctgaagtacccaagatagtcactcacctaaacaatcccaatcacttctggccaataaaaccaagcacacattctgagcttgaggacaaggtgggcatcaaccacatactttactcaatcaggaggaacttccctacactatttgatgaacacatatcccctttcctgtctgatttgaatatgctgaggctatcatgggttcagaggataaagttcttagacctttgtgtggcaattgatataacatcagaatgtcttggtattgttagtcatattataaaacatcgcagagaagaattgtatattgtaaagcaaaatgagcttgcaatgtcccatagcagagagtctcatccactggagagaggattcaaccttgaaccagaagaagtgtgcacaaatttcttgatacagattttgtttgagtcaatgttggtgcctgtgattatgtcaacatctcagttcaagaaatatttttggtttggtgagcttgaattgttgccaaacaatgcacaacatgatctgaaacaattaacccagttcatttgtgactgcaaaaagaataacacttctaggacaatgaatctggatgacttggatgttggctttgtaagcagcaaactgattttgtcctgtgttaatttgaacattagtgtatttattaatgaacttgactgggtgaatagagataactatgagaacattgaacaactcattttagcatcaccttctgaggtcattcccattgagctcaacctaacattttctcataagagagtgagccacaaatttagatatgagaggtctaccaattacatattaaaattaagatttcttattgaaagggagtcattgctggactcattggactctgatggttacctattgttgaacccccattctgttgagtattatgtttctcaatcttccgggaatcacataagtctggatggtgtgtcacttttggttctgaatcctctcatcaacggaaaagatgttctagacttcaatgatctattggaaggtcaagacattcacttcaagtcaaggtcaactgtgttccagaaggtgagaatagatttgaaaaatcgtttcaaggatttgaaaaacaaattttcctataagttgattggtcctgatgttgggatgcaacccctcattttagagggtgggttgatcaaagagggcaacagagtggtttctagattggaagtgaacttagactcaaaggtggtcatcattgcactggaagcattagaacctgagaaaaggccaagatttattgccaacctttttcagtacctttcaagtgcacagtctcacaacaagggaataagtatgaacgagcaggatttaagactcatgattgaaaatttccccgaagtgtttgaacacatgttgcatgatgccaaagattggttgaattgtggtcactttagtataataagaagtaagactctaggatctgttatgattgcagacgagacaggacccttcaagatcaagggaatcaggtgtcgaaagttgtttgaggacaatgagtcagtggagatcgagtaaacaaagagacggctagc

SEQ ID NO: 4 - DNA, 1207 bp, Artificial Sequence
  PIC-L-GFP-Bsm Modified Representative cDNA of modified L segment of Pichinde virus strain Munchique CoAn4763 isolate P18 (Genbank accession number EF529747.1)
gcgcaccgaggatcctaggcatttcttgatcagagacgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgtccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaacgtctctacaaccggccccatggggccgggggcccccgggcgcacccccggaggggggtgcgcccaggggccctggtttatggctcgtagggtggtgcagaggggctttctaggaactccatcttggttggcagtgagtggccgcatatctcgcagagattgcctctggagtgcattttggttaagcacccaagacacagatagtggtctttgcacctgatgagacctttgttgacgaaccagcaagatttgcagttgaacctgccatacaggccctgtggtagattgagggtcatggggacccttcccaccacatcttcgtcgccatgtctcttcctgacctctttgctatatctgagtcccatcccaagttttgaccaggacttgagctgggttcaaattggacaaatttgaagcgtgacccaaagatgcctaggatccccggtgcg

SEQ ID NO: 5 - DNA, 965 bp, Artificial Sequence
  PIC-miniS-GFP Modified Representative cDNA of a modified S segment cDNA of Pichinde virus strain Munchique CoAn4763 isolate P18 (Genbank accession number EF529746.1)
gcgcaccggggatcctaggcataccttggacgcgcatattacttgatcaaagagagacgaggcctcgtctctgccctagcctcgacatgggcctcgacgtcactccccaataggggagtgacgtcgaggcctctgaggacttgagcatgtcttcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggacgaagcactgcacgccgtaggtcaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgaagacattttggggttgtttgcacttcctccgagtcagtgaagaagtgaacgtacagcgtgatctagaatcgcctaggatccactgtgcg

SEQ ID NO: 6 - DNA, 1722 bp, Artificial Sequence
  NP-delta-BbsI  Modified Representative cDNA of a modified NP ORF of Pichinde virus strain Munchique CoAn4763 isolate P18 (Genbank accession number EF529747.1)
gaattcgaagacatcaaaatgtctgacaacatcccatcattccgctgggtacagtcccttaggaggggtctatccaactggacccatcctgtgaaggctgatgtgttgtcggacacaagagcactgttatctgctcttgactttcacaaagttgctcaagttcaaagaatgatgcgcaaagataaaaggactgattctgatctgaccaagttaagagacatgaacaaagaggttgatgctctgatgaatatgagatcaatccagagggacaatgtgcttaaggtgggaggcttagccaaagaggagctaatggagcttgcatctgatttggacaagttaagaaagaaagtcactagaactgagagtttgtctcagcctggtgtttatgggggcaatctcacaaacactcagttggaacaaagagccgaaatccttcgctcaatggggttcgctaatgctagacccacaggcaacagagatggggttgtgaagatctgggacatcaaggataatacattgttgatcaatcaatttggatcaatgccagccttaaccatcgcttgtatgactgagcaagggggtgaacaacttaatgatgttgtccaagcgctgagtgcacttggtttgctctacactgtcaagttcccgaacatgacagatctagagaaactcacacagcaacacagtgccctaaaaatcattagtaatgagccatcagccataaacatctcagggtacaatctcagtttgtctgcagcagtcaaagcagctgcttgcatgattgatggtggcaatatgcttgagaccatccaggtgaagccttctatgtttagtactctcataaagagtctattgcaaataaagaatcgtgaaggtatgtttgtgagcactacacccggacagagaaatccttatgaaaatttactatacaagatttgtctttcaggggatggttggccttacattggctcaaggtctcaagttcaagggagggcttgggataacaccactgtagatttagattcgaagccgagtgctatccagccaccagtaagaaacggaggatcaccggaccttaaacaaatccctaaggagaaagaagatactgttgtgtcctcaattcagatgcttgattcaaaagctaccacatggattgacattgaaggaacaccaaatgatccggtggaaatggccatctaccagcctgacacgggcaactacatacattgttacagatttccccacgatgagaagtccttcaaagagcaaagcaagtactcacatggtctccttttaaaggacttggctgatgcccaaccaggcctgatttcctcaatcatcagacatttacctcaaaacatggttttcactgctcaaggttcagatgatataatcagtttgttcgaaatgcatgggagaagagacttaaaagtgcttgacgtgaaactcagtgccgagcaagcacgcacctttgaggatgagatctgggagagatacaatctactctgcaccaaacataaaggtttggtcataaagaagaagaagaagggggctgcacaaaccactgcgaatcctcactgtgcattgcttgataccatcatgtttgatgcaacagtgacaggctgggttagggaccagaagccgatgagatgcttgcctattgacacgctgtacaggaacaacacagatctgatcaacctctgagctcatgtcttcgctagc

SEQ ID NO: 7 - DNA, 1915 bp, Artificial Sequence
  PIC-NP-Bsm  Representative cDNA obtained when NP-delta-BbsI was digested with BbsI to insert the BbsI-mutated NP ORF into the equally digested pol-I-PIC-miniS-GFP backbone, thereby replacing the GFP ORF with the NP ORF
gcgcaccggggatcctaggcataccttggacgcgcatattacttgatcaaagagagacgaggcctcgtctctgccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 8 - DNA, 1756 bp, Artificial Sequence
  PIC-GP-Bsm Representative cDNA obtained when GP-delta-BbsI was digested with BbsI to insert the BbsI-mutated GP ORF into the equally digested pol-I-PIC-miniS-GFP backbone, thereby replacing the GFP ORF with the GP ORF.
gcgcaccggggatcctaggcataccttggacgcgcatattacttgatcaaagagagacgaggcctcgtctctgccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 9 - DNA, 742 bp, Artificial Sequence
  GFP-Bsm Green fluorescent protein(GFP) synthesized with flanking BsmBI sites
cgtctctaaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgtccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcccagagacg

SEQ ID NO: 10 - DNA, 1261 bp, Artificial Sequence
  sP1AGM-Bsm Fusion protein consisting of the vesicular stomatitis virus glycoprotein (VSVG) signal peptide; the P1A antigen of the P815 mouse mastocytoma tumor cell line, a GSG linker, an enterovirus 2A peptide and mouse GM-CSF synthesized with flanking BsmBI sites
cgtctctaaggatgaaatgcctcctctaccttgcatttctcttcattggagtcaactgcatgagtgacaacaagaagcctgacaaggcccactctggcagtggaggagatggtgatggcaacagatgcaacctgctgcacagatacagcctggaagagatcctgccctacctgggctggctggtgtttgctgtggtgacaacaagcttcctggccctgcagatgttcattgatgccctgtatgaggaacagtatgagagggatgtggcctggattgccagacagagcaagagaatgagcagtgtggatgaggatgaggatgatgaggatgatgaagatgactactatgatgatgaggatgatgatgatgatgccttctatgatgatgaggatgatgaagaggaagaactggaaaacctgatggatgatgagtctgaggatgaggctgaggaagagatgagtgtggaaatgggggctggggcagaagagatgggagcaggtgccaactgtgcttgtgtgccaggacaccacctgagaaagaatgaagtgaagtgcaggatgatctacttcttccatgaccccaactttctggtgtccatccctgtgaaccccaaagaacagatggaatgcagatgtgagaatgcagatgaagaggtggccatggaagaagaagaggaagaggaagaagaagaagaagaggaagaaatgggcaacccagatggcttcagccctggaagtggtcaccatcaccaccatcatggcagtggggcaaccaacttcagcctgctgaaacaggctggggatgtggaagaaaatcctggccccatgtggctccagaatctgctttttctgggcattgtggtttacagcctgagtgcacccacaagatctcccatcacagtgacaagaccttggaagcatgtggaagcaatcaaagaggccctgaatctgcttgatgacatgccagtgaccctgaatgaagaagtggaagtggtgtcaaatgagttcagcttcaaaaaactgacctgtgtgcagaccaggctgaaaatttttgaacagggcctgagaggaaacttcacaaagctgaagggagctctgaacatgactgccagctactaccagacctactgcccccccaccccagagacagattgtgagacacaagtgaccacctatgctgacttcattgacagcctgaaaaccttcctgactgacatcccctttgagtgcaagaaacctgtgcagaagtgaagaaagagacg

SEQ ID NO: 11 - DNA, 2615 bp, Artificial Sequence
  PIC-NP-GFP
gcgcaccggggatcctaggcataccttggacgcgcatattacttgatcaaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgtccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 12 - DNA, 3131 bp, Artificial Sequence
  PIC-NP-sP1AGM
gcgcaccggggatcctaggcataccttggacgcgcatattacttgatcaaagatgaaatgcctcctctaccttgcatttctcttcattggagtcaactgcatgagtgacaacaagaagcctgacaaggcccactctggcagtggaggagatggtgatggcaacagatgcaacctgctgcacagatacagcctggaagagatcctgccctacctgggctggctggtgtttgctgtggtgacaacaagcttcctggccctgcagatgttcattgatgccctgtatgaggaacagtatgagagggatgtggcctggattgccagacagagcaagagaatgagcagtgtggatgaggatgaggatgatgaggatgatgaagatgactactatgatgatgaggatgatgatgatgatgccttctatgatgatgaggatgatgaagaggaagaactggaaaacctgatggatgatgagtctgaggatgaggctgaggaagagatgagtgtggaaatgggggctggggcagaagagatgggagcaggtgccaactgtgcttgtgtgccaggacaccacctgagaaagaatgaagtgaagtgcaggatgatctacttcttccatgaccccaactttctggtgtccatccctgtgaaccccaaagaacagatggaatgcagatgtgagaatgcagatgaagaggtggccatggaagaagaagaggaagaggaagaagaagaagaagaggaagaaatgggcaacccagatggcttcagccctggaagtggtcaccatcaccaccatcatggcagtggggcaaccaacttcagcctgctgaaacaggctggggatgtggaagaaaatcctggcccctggctccagaatctgctttttctgggcattgtggtttacagcctgagtgcacccacaagatctcccatcacagtgacaagaccttggaagcatgtggaagcaatcaaagaggccctgaatctgcttgatgacatgccagtgaccctgaatgaagaagtggaagtggtgtcaaatgagttcagcttcaaaaaactgacctgtgtgcagaccaggctgaaaatttttgaacagggcctgagaggaaacttcacaaagctgaagggagctctgaacatgactgccagctactaccagacctactgcccccccaccccagagacagattgtgagacacaagtgaccacctatgctgacttcattgacagcctgaaaaccttcctgactgacatcccctttgagtgcaagaaacctgtgcagaagtgagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg

SEQ ID NO: 13 - DNA, 2456 bp, Artificial Sequence
  PIC-GP-GFP
gcgcaccggggatcctaggcataccttggacgcgcatattacttgatcaaagatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttgacctacggcgtgcagtgcttcgtccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 14 - DNA, 2972 bp, Artificial Sequence
  PIC-GP-sP1AGM
gcgcaccggggatcctaggcataccttggacgcgcatattacttgatcaaagatgaaatgcctcctctaccttgcatttctcttcattggagtcaactgcatgagtgacaacaagaagcctgacaaggcccactctggcagtggaggagatggtgatggcaacagatgcaacctgctgcacagatacagcctggaagagatcctgccctacctgggctggctggtgtttgctgtggtgacaacaagcttcctggccctgcagatgttcattgatgccctgtatgaggaacagtatgagagggatgtggcctggattgccagacagagcaagagaatgagcagtgtggatgaggatgaggatgatgaggatgatgaagatgactactatgatgatgaggatgatgatgatgatgccttctatgatgatgaggatgatgaagaggaagaactggaaaacctgatggatgatgagtctgaggatgaggctgaggaagagatgagtgtggaaatgggggctggggcagaagagatgggagcaggtgccaactgtgcttgtgtgccaggacaccacctgagaaagaatgaagtgaagtgcaggatgatctacttcttccatgaccccaactttctggtgtccatccctgtgaaccccaaagaacagatggaatgcagatgtgagaatgcagatgaagaggtggccatggaagaagaagaggaagaggaagaagaagaagaagaggaagaaatgggcaacccagatggcttcagccctggaagtggtcaccatcaccaccatcatggcagtggggcaaccaacttcagcctgctgaaacaggctggggatgtggaagaaaatcctggcccctggctccagaatctgctttttctgggcattgtggtttacagcctgagtgcacccacaagatctcccatcacagtgacaagaccttggaagcatgtggaagcaatcaaagaggccctgaatctgcttgatgacatgccagtgaccctgaatgaagaagtggaagtggtgtcaaatgagttcagcttcaaaaaactgacctgtgtgcagaccaggctgaaaatttttgaacagggcctgagaggaaacttcacaaagctgaagggagctctgaacatgactgccagctactaccagacctactgcccccccaccccagagacagattgtgagacacaagtgaccacctatgctgacttcattgacagcctgaaaaccttcctgactgacatcccctttgagtgcaagaaacctgtgcagaagtgagccctagcctcgacatgggcctcgacgtcactccccaataggggagtgacgtcgaggcctctgaggacttgagcttatttacccagtctcacccatttgtagggtttctttgggattttataatacccacagctgcaaagagagttcctagtaatcctatgtggcttcggacagccatcaccaatgatgtgcctatgagtgggtattccaactaagtggagaaacactgtgatggtgtaaaacaccaaagaccagaagcaaatgtctgtcaatgctagtggagtcttaccttgtctttcttcatattcttttatcagcatttcattgtacagattctggctctcccacaaccaatcattcttaaaatgcgtttcattgaggtacgagccattgtgaactaaccaacactgcggtaaagaatgtctccctgtgatggtatcattgatgtaccaaaattttgtatagttgcaataagggattttggcaagctgtttgagactgtttctaatcacaagtgagtcagaaataagtccgttgatagtctttttaaagagattcaacgaattctcaacattaagttgtaaggttttgatagcattctgattgaaatcaaataacctcatcgtatcgcaaaattcttcattgtgatctttgttgcattttgccatcacagtgttatcaaaacattttattccagcccaaacaatagcccattgctccaaacagtaaccacctgggacatgttgcccagtagagtcactcaagtcccaagtgaaaaagccaaggagtttcctgctcacagaactataagcagttttttggagagccatccttattgttgccattggagtatatgtacagtgattttcccatgtggtgttctgtatgatcaggaaattgtaatgtgtcccaccttcacagtttgttagtctgcaagaccctccactacagttattgaaacattttccaacccacgcaatttttgggtccccaatgatttgagcaagcgacgcaataagatgtctgccaacctcacctcctctatccccaactgtcaagttgtactggatcaacaccccagcaccctcaactgttttgcatctggcacctacatgacgagtgacatggagcacattgaagtgtaactcattaagcaaccattttaatgtgtgacctgcttcttctgtcttatcacaattactaatgttaccatatgcaaggcttctgatgttggaaaagtttccagtagtttcatttgcaatggatgtgtttgtcaaagtgagttcaattccccatgttgtgttagatggtcctttgtagtaatgatgtgtgttgttcttgctacatgattgtggcaagttgtcaaacattcttgtgaggttgaactcaacgtgggtgagattgtgcctcctatcaatcatcatgccatcacaacttctgccagccaaaatgaggaaggtgatgagttggaataggccacatctcatcagattgacaaatcctttgatgatgcatagggttgagacaatgattaaggcgacattgaacacctcctgcaggacttcgggtatagactggatcaaagtcacaacttgtcccattttggggttgtttgcacttcctccgagtcagtgaagaagtgaacgtacagcgtgatctagaatcgcctaggatccactgtgcg

SEQ ID NO: 15 - DNA, 2472 bp, Artificial Sequence
  S-GP/GFPnat
gcgcaccggggatcctaggcataccttggacgcgcatattacttgatcaaagatgggacaagttgtgactttgatccagtctatacccgaagtcctgcaggaggtgttcaatgtcgccttaatcattgtctcaaccctatgcatcatcaaaggatttgtcaatctgatgagatgtggcctattccaactcatcaccttcctcattttggctggcagaagttgtgatggcatgatgattgataggaggcacaatctcacccacgttgagttcaacctcacaagaatgtttgacaacttgccacaatcatgtagcaagaacaacacacatcattactacaaaggaccatctaacacaacatggggaattgaactcactttgacaaacacatccattgcaaatgaaactactggaaacttttccaacatcagaagccttgcatatggtaacattagtaattgtgataagacagaagaagcaggtcacacattaaaatggttgcttaatgagttacacttcaatgtgctccatgtcactcgtcatgtaggtgccagatgcaaaacagttgagggtgctggggtgttgatccagtacaacttgacagttggggatagaggaggtgaggttggcagacatcttattgcgtcgcttgctcaaatcattggggacccaaaaattgcgtgggttggaaaatgtttcaataactgtagtggagggtcttgcagactaacaaactgtgaaggtgggacacattacaatttcctgatcatacagaacaccacatgggaaaatcactgtacatatactccaatggcaacaataaggatggctctccaaaaaactgcttatagttctgtgagcaggaaactccttggctttttcacttgggacttgagtgactctactgggcaacatgtcccaggtggttactgtttggagcaatgggctattgtttgggctggaataaaatgttttgataacactgtgatggcaaaatgcaacaaagatcacaatgaagaattttgcgatacgatgaggttatttgatttcaatcagaatgctatcaaaaccttacaacttaatgttgagaattcgttgaatctctttaaaaagactatcaacggacttatttctgactcacttgtgattagaaacagtctcaaacagcttgccaaaatcccttattgcaactatacaaaattttggtacatcaatgataccatcacagggagacattctttaccgcagtgttggttagttcacaatggctcgtacctcaatgaaacgcattttaagaatgattggttgtgggagagccagaatctgtacaatgaaatgctgataaaagaatatgaagaaagacaaggtaagactccactagcattgacagacatttgcttctggtctttggtgttttacaccatcacagtgtttctccacttagttggaatacccactcataggcacatcattggtgatggctgtccgaagccacataggattactaggaactctctttgcagctgtgggtattataaaatcccaaagaaaccctacaaatgggtgagactgggtaaataagccctagcctcgacatgggcctcgacgtcactccccaataggggagtgacgtcgaggcctctgaggacttgagcatgtcttcttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcgggtcttgaagttcaccttgatgccgttcttctgcttgtcggcggtgatatagaccttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggacgaagcactgcacgccgtaggtcaaggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatgaagacattttggggttgtttgcacttcctccgagtcagtgaagaagtgaacgtacagcgtgatctagaatcgcctaggatccactgtgcg

SEQ ID NO: 16 - DNA, 3422 bp, Artificial Sequence
  modified S-delta-BbsI-Pichinde virus strain Munchique CoAn4763 isolate P18 (Genbank accession number EF529746.1) segment S
gcgcaccggggatcctaggcataccttggacgcgcatattacttgatcaaagatgggacaagttgtgactttgatccagtctatacccgaagtcctgcaggaggtgttcaatgtcgccttaatcattgtctcaaccctatgcatcatcaaaggatttgtcaatctgatgagatgtggcctattccaactcatcaccttcctcattttggctggcagaagttgtgatggcatgatgattgataggaggcacaatctcacccacgttgagttcaacctcacaagaatgtttgacaacttgccacaatcatgtagcaagaacaacacacatcattactacaaaggaccatctaacacaacatggggaattgaactcactttgacaaacacatccattgcaaatgaaactactggaaacttttccaacatcagaagccttgcatatggtaacattagtaattgtgataagacagaagaagcaggtcacacattaaaatggttgcttaatgagttacacttcaatgtgctccatgtcactcgtcatgtaggtgccagatgcaaaacagttgagggtgctggggtgttgatccagtacaacttgacagttggggatagaggaggtgaggttggcagacatcttattgcgtcgcttgctcaaatcattggggacccaaaaattgcgtgggttggaaaatgtttcaataactgtagtggagggtcttgcagactaacaaactgtgaaggtgggacacattacaatttcctgatcatacagaacaccacatgggaaaatcactgtacatatactccaatggcaacaataaggatggctctccaaaaaactgcttatagttctgtgagcaggaaactccttggctttttcacttgggacttgagtgactctactgggcaacatgtcccaggtggttactgtttggagcaatgggctattgtttgggctggaataaaatgttttgataacactgtgatggcaaaatgcaacaaagatcacaatgaagaattttgcgatacgatgaggttatttgatttcaatcagaatgctatcaaaaccttacaacttaatgttgagaattcgttgaatctctttaaaaagactatcaacggacttatttctgactcacttgtgattagaaacagtctcaaacagcttgccaaaatcccttattgcaactatacaaaattttggtacatcaatgataccatcacagggagacattctttaccgcagtgttggttagttcacaatggctcgtacctcaatgaaacgcattttaagaatgattggttgtgggagagccagaatctgtacaatgaaatgctgataaaagaatatgaagaaagacaaggtaagactccactagcattgacagacatttgcttctggtctttggtgttttacaccatcacagtgtttctccacttagttggaatacccactcataggcacatcattggtgatggctgtccgaagccacataggattactaggaactctctttgcagctgtgggtattataaaatcccaaagaaaccctacaaatgggtgagactgggtaaataagccctagcctcgacatgggcctcgacgtcactccccaataggggagtgacgtcgaggcctctgaggacttgagctcagaggttgatcagatctgtgttgttcctgtacagcgtgtcaataggcaagcatctcatcggcttctggtccctaacccagcctgtcactgttgcatcaaacatgatggtatcaagcaatgcacagtgaggattcgcagtggtttgtgcagcccccttcttcttcttctttatgaccaaacctttatgtttggtgcagagtagattgtatctctcccagatctcatcctcaaaggtgcgtgcttgctcggcactgagtttcacgtcaagcacttttaagtctcttctcccatgcatttcgaacaaactgattatatcatctgaaccttgagcagtgaaaaccatgttttgaggtaaatgtctgatgattgaggaaatcaggcctggttgggcatcagccaagtcctttaaaaggagaccatgtgagtacttgctttgctctttgaaggacttctcatcgtggggaaatctgtaacaatgtatgtagttgcccgtgtcaggctggtagatggccatttccaccggatcatttggtgttccttcaatgtcaatccatgtggtagcttttgaatcaagcatctgaattgaggacacaacagtatcttctttctccttagggatttgtttaaggtccggtgatcctccgtttcttactggtggctggatagcactcggcttcgaatctaaatctacagtggtgttatcccaagccctcccttgaacttgagaccttgagccaatgtaaggccaaccatcccctgaaagacaaatcttgtatagtaaattttcataaggatttctctgtccgggtgtagtgctcacaaacataccttcacgattctttatttgcaatagactctttatgagagtactaaacatagaaggcttcacctggatggtctcaagcatattgccaccatcaatcatgcaagcagctgctttgactgctgcagacaaactgagattgtaccctgagatgtttatggctgatggctcattactaatgatttttagggcactgtgttgctgtgtgagtttctctagatctgtcatgttcgggaacttgacagtgtagagcaaaccaagtgcactcagcgcttggacaacatcattaagttgttcacccccttgctcagtcatacaagcgatggttaaggctggcattgatccaaattgattgatcaacaatgtattatccttgatgtcccagatcttcacaaccccatctctgttgcctgtgggtctagcattagcgaaccccattgagcgaaggatttcggctctttgttccaactgagtgtttgtgagattgcccccataaacaccaggctgagacaaactctcagttctagtgactttctttcttaacttgtccaaatcagatgcaagctccattagctcctctttggctaagcctcccaccttaagcacattgtccctctggattgatctcatattcatcagagcatcaacctctttgttcatgtctcttaacttggtcagatcagaatcagtccttttatctttgcgcatcattctttgaacttgagcaactttgtgaaagtcaagagcagataacagtgctcttgtgtccgacaacacatcagccttcacaggatgggtccagttggatagacccctcctaagggactgtacccagcggaatgatgggatgttgtcagacattttggggttgtttgcacttcctccgagtcagtgaagaagtgaacgtacagcgtgatctagaatcgcctaggatccactgtgcg